(19) United States
(12) Patent Application Publication
(10) Pub. No.: US 2007/0178491 A1
(43) Pub. Date: Aug. 2, 2007

(54) Compositions and Methods for Synthesizing Nucleic Acids

(75) Inventors: Kyusung Park, Vista, CA (US); Jun E. Lee, San Diego, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/558,294

(22) Filed: Nov. 9, 2006

Related U.S. Application Data
(63) Continuation of application No. 10/655,579, filed on Sep. 5, 2003, now abandoned.
(60) Provisional application No. 60/408,609, filed on Sep. 5, 2002.
(60) Provisional application No. 60/427,867, filed on Nov. 19, 2002.

(51) Int. Cl. C12Q 1/68; C12P 19/34; C07K 16/40

(52) U.S. Cl. 435/6; 435/91.2; 530/388.26

(57) ABSTRACT

Disclosed are compositions preferably for use in nucleic acid synthesis that include one or more anti-reverse transcriptase (RT) antibodies and/or one or more anti-DNA polymerase (DNAP) antibodies and/or single strand binding proteins (SSBs). Some of the disclosed compositions include one or more anti-DNAP antibodies and/or one or more anti-RT antibodies and one or more SSBs. Other disclosed compositions include two or more SSBs. The disclosed nucleic acid synthesis compositions also can include one or more DNAPs, one or more RTs, one or more nucleotides, one or more primers, and/or one or more templates. Also disclosed are methods for using such compositions in nucleic acid synthesis, amplification and sequencing, where various combinations of anti-RT antibodies, anti-DNAP antibodies and/or SSBs can improve the yield and/or homogeneity of primer extension products.

### CROSS-REFERENCE TO RELATED APPLICATIONS

The present application claims the benefit of the filing dates of U.S. Provisional Appl. Nos. 60/408,609, filed Sep. 5, 2002, and 60/427,867, filed Nov. 19, 2002, the disclosures of both of which are incorporated herein by reference in their entireties.

### STATEMENT REGARDING FEDERALLY SPONSORED RESEARCH OR DEVELOPMENT

Not applicable.

### REFERENCE TO SEQUENCE LISTING/TABLE/COMPUTER PROGRAM LISTING APPENDIX(SUBMITTED ON A COMPACT DISC AND AN INCORPORATION-BY-REFERENCE OF THE MATERIAL ON THE COMPACT DISC)

Not applicable.

### BACKGROUND OF THE INVENTION

1. Field of the Invention

This invention relates to methods and materials useful for nucleic acid synthesis (e.g., polymerase chain reaction-based nucleic acid synthesis).

2. Related Art

DNA polymerases (DNAPs) synthesize DNA molecules that are complementary to all or a portion of a nucleic acid template (preferably a DNA template). Upon hybridization of a primer to a DNA template to form a primed template, DNA polymerases can add nucleotides to the 3′ hydroxy end of the primer in a template dependent manner (i.e., depending upon the sequence of nucleotides in the template). Thus, in the presence of deoxyribonucleoside triphosphates (dNTPs) and a primer, a new DNA molecule, complementary to all or a portion of one or more nucleic acid templates, can be synthesized.

DNAPs have been used to detect nucleic acids in biological and environmental test samples, e.g., using polymerase chain reaction (PCR)-based nucleic acid synthesis (see e.g., U.S. Pat. Nos. 4,683,195; 4,683,202 and 4,965,188). In PCR-based nucleic acid synthesis, one or more templates are hybridized to smaller complementary “primer” nucleic acids in the presence of a thermostable DNAP and deoxyribonucleoside triphosphates. Upon hybridization of a primer and a template to form a “primed template complex,” DNAP can extend the primer in a template directed manner to yield a primer extension product. Primer extension products can then serve as templates for nucleic acid synthesis. Upon denaturation, the primer extension products can hybridize with primers to form primed template complexes that can serve as DNAP substrates. Cycles of hybridization, primer extension and denaturation can be repeated many times to exponentially increase the number of primer extension products. Thus, PCR-based nucleic acid synthesis is a very sensitive technique for detecting template nucleic acids.

The yield and homogeneity of primer extension products made by DNAP can be adversely affected by “mispriming” (i.e., hybridization of primers to inappropriate regions of the template, or to non-template nucleic acids). Primers are designed to hybridize to a specific region of a template nucleic acid. Mispriming can occur when nucleic acid synthesis mixtures containing template, primers, DNAP and nucleotides are maintained at lower temperatures (e.g., during manufacture, shipping, or storage). Extension of misprimed nucleic acids can obscure properly primed primer extension products (i.e., produce high background). In addition, diversion of nucleic acid synthesis reaction constituents to extend misprimed nucleic acids can reduce the yield of properly primed primer extension products, reducing the sensitivity of detection.

### BRIEF SUMMARY OF THE INVENTION

The invention features compositions and methods for synthesizing nucleic acids. The methods and materials of the invention can enhance the yield and/or homogeneity of primer extension products made by DNAPs.

In one aspect, the compositions and methods of the invention use or incorporate one or more (e.g. one, two, three, four, five, six, etc.) single strand DNA binding proteins (SSBs).

In another aspect, the compositions and methods of the invention use or incorporate one or more anti-DNAP antibodies and/or one or more anti-reverse transcriptase (RT) antibodies.

In yet another aspect, the compositions and methods of the invention use or incorporate one or more SSBs and one or more anti-DNAP antibodies.

In yet another aspect, the compositions and methods of the invention use or incorporate one or more SSBs and one or more anti-RT antibodies.

Preferred compositions and methods may use or incorporate, in addition to SSBs and/or anti-DNAP antibodies and/or anti-RT antibodies, one or more templates, one or more nucleotides, one or more vectors, one or more ligases, one or more topoisomerases, one or more primers, one or more nucleic acid molecules, one or more buffers or buffering salts, one or more RTs, and one or more DNAPs.

The invention also relates to kits (preferably kits for use in carrying out the methods of the invention). Such kits may include one or more SSBs and/or anti-DNAP antibodies and/or anti-RT antibodies. The kits of the invention may also include one or more components selected from the group consisting of one or more host cells (which preferably are competent to take up nucleic acid molecules), one or more templates, one or more nucleotides, one or more nucleic acid molecules, one or more primers, one or more vectors, one or more ligases, one or more topoisomerases, one or more buffers or buffering salts, one or more RTs, one or more DNAPs, and directions or protocols for carrying out any method of the invention.

The compositions of the invention preferably are used in nucleic acid synthesis reactions, or are generated during nucleic acid synthesis reactions. The methods of the invention preferably are used to synthesize one or more nucleic acid molecules. Thus, the invention may be used in amplifying nucleic acid molecules (for example by PCR), in reverse transcription of nucleic acid molecules (e.g. cDNA synthesis), and in coupled or uncoupled reverse transcription/amplification reactions (e.g. RTPCR).

Other features and advantages of the invention will be apparent from the following detailed description, and from the claims. The disclosed materials, methods, and examples are illustrative only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the art to which this invention belongs. All publications, patent applications, patents, and other references mentioned herein are incorporated by reference in their entirety. In case of conflict, the present specification, including definitions, will control.

### BRIEF DESCRIPTION OF THE DRAWINGS

FIG. 1: SDS PAGE analysis for samples from intermediate steps in purification of AccuPrime protein. The protein purified (lane 4) was shown to contain less contaminating proteins, compared to the control protein (lane 5): Lane 1, bacterial lysate containing over-expressed AccuPrime protein; Lane 2, pool of fractions under a peak containing AccuPrime protein from Ni-NTA agarose column; Lane 3a, pool of fractions under a peak containing AccuPrime protein from ssDNA agarose column; Lane 3b, pool of fractions immediately following the major peak containing AccuPrime protein from ssDNA agarose column; Lane 4, pool of fractions under a peak containing AccuPrime protein from Mono Q (5/5) column; Lane 5, control AccuPrime protein obtained from UC Davis group.

FIG. 2: SDS PAGE comparison of AccuPrime protein preparations purified by modified protocols. Modified protocol contains protease inhibitor cocktail in buffers for the column chromatography. An extensive wash at Ni-NTA column step yields the protein as pure as the control using just one column: Lane 1, AccuPrime protein eluted from Ni-NTA agarose column after extensive wash (10 column volume wash); Lane 2, AccuPrime protein eluted from Ni-NTA agarose column after moderate wash (5 column volume wash); Lane 3, control AccuPrime protein from UC, Davis.

FIG. 3: Endo-nuclease activity assay for AccuPrime protein preparation (lots 3 and 4). The assay checks the extent of conversion of super-coiled circular dsDNA to relaxed circular molecule: Lanes 1 and 6, control DNA (φX174) alone; Lanes 2 and 3, φX174 DNA incubated with 10× and 20× of control AccuPrime protein, respectively; Lanes 4 and 5, φX174 DNA incubated with 10× and 20× of AccuPrime protein (lot 3), respectively; Lanes 7 and 8, φX174 DNA incubated with 10× and 20× of AccuPrime protein (lot 4), respectively; Lanes 9 to 15, duplication of lanes, 1, 2, 3, 4, 5, 7 and 8, respectively, with twice as much samples loaded. Lanes 2, 3, 10 and 11 show AccuPrime protein binding to super-coiled DNA.

FIG. 4: Exo-nuclease activity assay for AccuPrime protein preparation (lots 3 and 4). The exo-nuclease assay was done at 72° C. for 30 min. for and internal exonuclease activity. The assay shows no detectable nuclease activity. Incubation at 37° C. resulted in similar gel showing no degradation products.

FIG. 5: Electrophoretic mobility shift assay (EMSA) for AccuPrime protein with 86-mer. Specified amounts of AccuPrime protein were added to the oligonucleotides, incubated at 70° C. for 5 min, and an aliquot from reaction was loaded on the 6% non-denaturing polyacrylamide gel with the currents on. The electrophoresis was done at 100V for 1 hr, and the gel was dried and autoradiographed. The gel showed super-shift above the shifted band indicating second protein binding to an oligonucleotide molecule. As protein concentration increased, the intensity of the shift increased while super-shift remained same indicating negative cooperativity.

FIG. 6: Unit assay for Taq DNA polymerase in the presence of SSB (AccuPrime protein or E. coli SSB) under various conditions. Unlike E. coli SSB which shows general tendency of inhibition as the protein concentration increases, AccuPrime protein enhances Taq DNA polymerase unit activity in a concentration-dependent manner where the optimal enhancement is achieved under a sub-optimal condition for the polymerase at AccuPrime protein concentration of 0.1 mg/50 ml reaction.

FIG. 7: The temperature dependency of the unit activity enhancement of Taq DNA polymerase by AccuPrime protein. The temperature dependent enhancement shows a three phases: first, temperature independent phase up to 65° C.; second, directly proportional to temperature with the maximum at 70° C.; and third, inversely proportional to temperature over 70° C.

FIG. 8: Scan profile of alkaline agarose gel electrophoresis for primer extension products by Taq DNA polymerase using a specific primer and single stranded circular M13mp19 DNA as a template, in the presence or the absence of AccuPrime protein: (A) primer extension in the absence of AccuPrime protein; (B) with 50 ng AccuPrime protein/50 ml r×n; (C) with 100 ng AccuPrime protein/50 ml rxn; and, (D) with 100 ng MthSSB/50 ml r×n. Results show that in the presence of 100 ng of AccuPrime protein in 50 ml rxn, the peak population of extension products shifted toward lower molecular weight indicating the polymerase extending the primer shorter in the presence of AccuPrime protein than those in the control. This phenomenon was most obvious at 1.5 min time point. The second peak showing on top of the gel in the bottom panels (C and D) is the primer from the top panel.

FIG. 9: Accelerated stability assay for 10× AccuPrime Taq PCR Reaction Mix and 2× AccuPrime Taq PCR Super Mix. The arrow on the right indicates the specific product expected from the primer set. It is shown that even after incubation equivalent to storage at −20° C. for half a year (2 days at 42° C.) and a year (4 days at 42° C.) reaction mixes function as well as those in control. The gel was also showing that nucleotide mixes used to formulate the mixes was not functioning properly, since external source of nucleotide added performed fine while those stored at −20° C. with nucleotide performed as poorly.

FIG. 10: Real-time stability assay for AccuPrime Taq PCR Reaction Mixes and SuperMixes up to 6 month at room temperature. Each sample was duplicated: Panel A Lanes 1 and 12, Platinum Taq DNA polymerase control; Lane 2, AccuPrime Taq PCR Reaction Mix I (RMI) control; Lanes 3-6, RMI after incubation at RT for 1, 2, 3 and 6 month, respectively; Lane 7, AccuPrime Taq PCR Reaction Mix I without glycerol (RMI-gly) control; Lanes 8-11 RMI-gly after incubation at RT for 1, 2, 3 and 6 month, respectively; Lane 13, AccuPrime Taq PCR SuperMix I (SMI) control; Lanes 8-11 SMI after incubation at RT for 1, 2, 3 and 6 month, respectively. Panel B shows counter parts of AccuPrime Taq PCR Reaction Mix II with and without glycerol, and AccuPrime Taq PCR SuperMix II as shown in Panel A.

FIG. 11: TOPO TA cloning with PCR amplification products from AccuPrime Taq DNA polymerase. The PCR amplification products using two different primer sets were cloned into pCR2.1 TOPO vector, transformed TOP10 cells and selected 6 transformants randomly from each transformation. Plasmids purified from the transformants were checked for the right insert and sequenced the flanking region to make sure they were flanked by TT at 5′end and AA at 3′ end. The sequencing showed the right insert flanked by TT and AA (blue arrows) indicating AccuPrime Taq DNA polymerase adds 3′ A overhang necessary for TOPO TA cloning.

FIG. 12: Restriction enzyme digestion assay for amplification products from AccuPrime Taq DNA polymerase mediated PCR. PCR was done with either 50 or 200 ng of genomic DNA template (K562) for reaction. After PCR, 5 to 10 μl of amplification reaction mix were used directly in 20 μl restriction digestion reaction. Two different digestions shows no detectable hindrance from the components carried over from the PCR mix.

FIG. 13: Comparison of PCR performance of AccuPrime Taq with Taq alone and Hot Star Taq (Qiagen) using four different primer set covering the sizes of amplicons from 1 to 4.4 kb. AccuPrime Taq out performed others in specificity as well as the yield of the specific products.

FIG. 14: Performance comparison of AccuPrime Taq DNA polymerase with Hot Star Taq (Qiagen) using two sets of primers based on the size of the amplicons; (A) β-globin, 468 bp; β-globin, 731 bp; c-myc, 822 bp; β-globin, 1100 bp; and Hpfh, 1,350 bp, (B) β-globin, 2.2 kb; and β-globin, 3.6 kb. AccuPrime Taq performed consistently with a high specificity regardless of the size of the amplicon up to 3.6 kb, while Hot Start Taq were more prone to produce non specific bands as the amplicon size increased.

FIG. 15: Discrimination against false priming site by AccuPrime Taq DNA polymerase, compared with Taq DNA polymerase or Hot Star Taq (Qiagen). A false priming site was introduced by 13 base homology in two different locations of the template, separated by 350 bp, where 13 nucleotides of the 3′ end of the reverse primer could anneal to. The remaining 7 nucleotides of the 20 nucleotide long reverse primer anneals only to the genuine priming site (13951). Only the AccuPrime Taq discriminated against the 13 base homology priming while maintaining a high yield.

FIG. 16: Schematic presentation of the mechanism of MjaSSB in PCR reaction. In it, represents Taq DNA polymerase, anti-Taq DNA polymerase antibody, heat-denatured antibody, AccuPrime protein,

- DNA molecule, primer, non-specifically annealed primer, and - - - newly synthesized DNA. This schematics depicts MjaSSB functions as stabilizer for specific primer-template complex, as competitive inhibitor for non-specific primer annealing, and recruiter for Taq DNA polymerase to specifically primed sites.

FIG. 17: Feasibility assay for PCR miniaturization using AccuPrime Taq DNA polymerase. Unlike Taq DNA polymerase alone, AccuPrime Taq DNA polymerase functions efficiently regardless of the reaction volume and the amount of the enzyme itself could be lowered proportionally to the reaction volume without losing the robustness or specificity of the reaction.

FIG. 18: PCR miniaturization using AccuPrime Taq DNA polymerase with 5 ng human genomic DNA (K562) per reaction as a template. The primers were designed to amplify 1013 bp long amplicon. Unlike Platinum Taq DNA polymerase, AccuPrime Taq DNA polymerase functions efficiently regardless of the reaction volume and the amount of the enzyme itself could be lowered proportionally to the reaction volume without losing the robustness or specificity of the reaction.

FIG. 19: PCR amplification of a difficult template (70% GC) with increasing amount of AccuPrime protein added to Taq DNA polymerase or Platinum Taq DNA polymerase. Marked improvement on the yield of specific product is shown with Taq DNA polymerase at 1× AccuPrime Protein concentration (400 ng/50 ml rxn). Platinum Taq DNA polymerase alone performed better than Taq DNA polymerase alone, but the addition of AccuPrime protein was necessary to amplify the specific product with a high specificity.

FIG. 20: PCR amplification of a difficult template (GC rich) in combination with PCRx enhancer solution. Marked improvement on the yield of specific product is shown with AccuPrime Taq DNA polymerase even at 1× PCRx concentration. With Platinum Taq, at least 3× PCRx was required to see any enhancement on the specificity.

FIG. 21: Genotyping using PCR amplification for gender-specific genes as target amplicons Both the genes, SRY and DYS-391, reside in Y chromosome so that the genomic DNA only from male would have the specific targets. In both cases AccuPrime Taq (AP Taq) showed specific amplification product while suppressing background. The control HotStar Taq (HS Taq) showed many non-specific products in the background especially in SRY gene.

FIG. 22: Multiplex PCR for 12 sets of primers with 100 ng of human genomic DNA as a template in 50 ml reaction. Full 12 amplicons were amplified with 5 units of AccuPrime Taq DNA polymerase. However, the yields (band intensities) were consistent among amplicons as the number of amplicons increased, indicating a robust multiplex PCR application with AccuPrime Taq with little optimization for individual primer set.

FIG. 23: Multiplex PCR for 20 sets of primers with varying amounts of the polymerase. Full 20 amplicons were amplified either with 2, 5 or 10 units of Taq or AccuPrime Taq. However, the variation in band intensities (yields) among amplicons was less with AccuPrime Taq, indicating a robust multiplex PCR with AccuPrime Taq with less optimization required.

FIG. 24: Comparison of High Throughput screening between Platinum Taq and AccuPrime Taq. Colonies from a plate incubated overnight was “picked” by pipette tips and mixed with PCR reaction mixes for a 18 cycle PCR. Only AccuPrime Taq DNA polymerase could successfully amplify the specific amplicon in more than 90% of the 96 colonies. High sensitivity of AccuPrime Taq DNA polymerase made this type of high throughput application possible.

FIG. 25: Performance comparison of AccuPrime Taq DNA polymerase with Taq DNA polymerase and other hot start polymerases (AmpliTaq Gold, Perkin Elmer; Jump Start, Sigma; Fast Start, Roche; Hot Star, Qiagen; Sure Start, Stratagene) using 6 primer sets with amplicons ranging from 264 to 4,350 bp (Pr 1.3, 264 bp; Rhod, 646 bp; β-globin, 731 bp; Hpfh, 1,350 bp; p53, 2,108 bp; p53, 4,350 bp). AccuPrime Taq shows the highest specificity and consistent yields regardless of the amplicon sizes. The yields from the AccuPrime Taq DNA polymerase are among the highest.

FIG. 26: Performance comparison of AccuPrime Taq DNA polymerase with AmpliTaq Gold (Perkin Elmer) in two step PCR (annealing and elongation in a single step following 94° C. denaturation) using 4 primer sets (Pr 1.3, 264 bp; Rhod, 646 bp; β-globin, 731 bp; Hpfh, 1,350 bp). AccuPrime Taq performed consistently with a high specificity and high yield regardless of the annealing temperatures, while AmpliTaq Gold required a narrow window of annealing temperature for each primer set. The result implies less optimization requirement for AccuPrime Taq, compared to AmpliTaq Gold.

FIG. 27: Elution Profile of EMD-SO₃ column chromatography for AccuPrime Protein II purification from BL21 (DE3) CodonPlus (Stratagene) strain. During wash and elution, 2.5 ml fractions were collected. Gradient elution from 50 to 650 mM NaCl followed by 650 mM NaCl elution separates contaminating proteins in a shoulder (Fractions 38 to 44), while gradient elution from 50 to 1000 mM salt co-elutes contaminants with the protein (see gel electrophoresis in FIG. 2).

FIG. 28: SDS polyacrylamide gel electrophoresis (Novex 4-20% Tris Glycine gel) for cross-column analysis of the fractions from Fractogel EMD-SO₃ column from BL21(DE3) CodonPlus host. Lanes in the gel contain: M) markers (BenchMark protein ladders, Invitrogen); 1) flow-through; 2) fraction#24 (2.5 ml fractions); 3) fraction#28; 4) fraction#32; 5) fraction#34; 6) fraction#38; 7) fraction#40; 8) fraction#42; 9) fraction#44; 10) fraction#48; and 11) fraction#52. The gel shows two peaks where AccuPrime protein II elute as a major component, where the first peak contain more contaminants than the second peak. In fact the purity of AccuPrime Protein II in the second peak was high enough to allow one-step purification from the host.

FIG. 29: SDS polyacrylamide gel electrophoresis (Novex 4-20% Tris Glycine gel) analysis for purification steps from the modified protocol. Lanes in the gel contain: M) markers; 1) lysate; 2) supernatant from heat treatment step; 3) load for EMD-SO₃ column; 4) flow through from EMD-SO₃ column; and 5) fraction pool under the second peak from EMD-SO₃ column. The gel shows almost complete retention of AccuPrime Protein II in EMD-SO₃ column, and 90 to 95% purity obtained by the column step, suggesting plausibility of one-step purification of the protein.

FIG. 30: SDS polyacrylamide gel electrophoresis (Novex 4-20% Tris Glycine gel) for cross-column analysis of the fractions from EMD-SO₃ column from BL21(DE3) host. Lanes in the gel contain: M) markers; 1) lysate; 2) heat supernatant; 3) flow-through; 4) wash with 50 mM NaCl; 5) fraction#29 (2.5 ml fractions); 6) #31; 7) #33; 8) #35; 9) #36; 10) #38; 11) #40; 12) #42; 13) #44; 14) #46; 15) #48; 16) #50; 17) #52; 18) #54; 19) #56; 20) #60; 21) #65; and 22) #69. The gel shows that while AccuPrime protein II elutes in two peaks as before (FIG. 2), the second peak still contains a considerable amount of contaminants.

FIG. 31: SDS polyacrylamide gel electrophoresis (Novex 4-20% Tris Glycine gel) for cross-column analysis of the fractions from CHT2-1 hydroxyapatite column from BL21(DE3) host. Lane M indicates the markers; 1) load (pool from EMD-SO₃); 2) flow-through; 3) wash with 50 mM Na phosphate; 4) #6 fractions (1 ml each) from linear gradient; 5) #8; 6) #9; 7) #11; 8) #13; 9) #15; 10) #20; and 11) #10 fraction from 500 mM Na phosphate elution. The gel shows majority of contaminants eluted during the wash step (lane 3), while AccuPrime protein II eluted during the gradient (lane 5).

FIG. 32: Endonuclease activity assay using supercoiled circular plasmid (φX174) incubated with varying amounts of AccuPrime proteins in 50 μl reaction solution at 37° C. for 1 hr. The resulting plasmid was mixed with 5 μl of 10× BlueJuice and analyzed on 0.8% agarose gels for appearance of relaxed circular or linear DNA. Lanes 1 to 4 were from samples made from commercial Platinum Pfx Amplification buffer with 0, 0.75 (2.5×), 1.5 (5×) and 3 (10×) μg of AccuPrime Protein II (APP II), respectively. Lanes 5 to 8 were identical to lanes 1 to 4, except all the components were assembled for the pilot lot. Lanes 9 to 12 contain AccuPrime Protein I (APP I) at the amount of 0, 0.5 (5×), 1 (10×) and 2 (20×) μg, respectively. Panel (A) samples were in 1× BlueJuice and loaded to the gel without heating. Panel (B) samples were heated at 95° C. for 5 min in 1× BlueJuice, and loaded on the gel. Panel (C) samples were heated at 95° C. for 5 min in 1× BlueJuice and 0.5% SDS, and loaded on the gel. The gels clearly show strong binding of AccuPrime Protein II that resulted in shift in mobility of the DNA band and resistant to heat treatment without SDS. AccuPrime protein I came off from the DNA upon heating at 95° C. for 5 min even without SDS.

FIG. 33: PCR functional assay for purified AccuPrime Protein II (APP II). PCR was done with Platinum Pfx in the presence or absence of AccuPrime proteins as indicated. The primer set (p53 2380 bp) targets human p53 gene and amplifies 2380 bp segment of the gene. The PCR product was mixed with BlueJuice and loaded on a 0.8% agarose gel for analysis. The intensity of the specific PCR product (indicated by an arrow) by Platinum Pfx was shown to be intensified as the amount of AccuPrime Protein II (APP II) increased in the presence of 100 ng of AccuPrime Protein I (APP I).

FIG. 34: Host DNA contamination assay in the preparation of AccuPrime Protein II. The assay was done by PCR using a primer set targeting a single copy gene in E. coli genome (priA) in the presence of denatured AccuPrime Protein II at 1× (300 ng per 50 μl reaction) or 2× (600 ng) concentration without added DNA template, with two different polymerases, Pfx and Taq DNA polymerases. Control reactions contain a known amount of E. coli genomic DNA serving as concentration markers in estimating the amount of contaminating DNA.

FIG. 35: Selected examples of PCR enhancement on Pfx DNA polymerase by adding both AccuPrime Protein I and AccuPrime Protein II to the reaction mixes. “Cont” indicates Platinum Pfx DNA polymerase control, “A” the Formula A (only AccuPrime Protein I), and “B” the Formula B (both AccuPrime Protein I and AccuPrime Protein II). The gels show that Formula A resulted in a limited enhancement in a few cases, such as Rhod_{—}670 and Rhod_{—}3831, while Formula B resulted in marked improvement in the yield, the specificity or both in some cases.

FIG. 36: Selected examples of PCR optimization through increasing Pfx amplification buffer concentration. The buffer concentration was increased in 0.5× increment up to 2.5×. A higher concentration of the buffer was proven to be inhibitory. As the gels show the increased buffer strength in some cases enhances overall performance independent of the formula (Platinum, Formula A or Formula B), in the other only the Formula B of AccuPrime Pfx, and in another Platinum and Formula A. However, it seems that titrating buffer strength would be an option to enhance PCR performance of AccuPrime Pfx DNA polymerase.

FIG. 37: Selected examples of PCR optimization through adding an additional component. PCR reaction was done with Hbg_{—}3.6 primer set for different optimization schemes for easy comparison. The Pfx Amplification buffer contains ammonium sulfate at 18 mM, therefore 45 mM ammonium sulfate would be equivalent of 2.5× of the buffer. KCl is a completely new component for Pfx but generally used in Taq PCR buffer. As the gels show the additional component could enhance overall performance of AccuPrime Pfx. This provides an alternative option to enhance PCR performance of AccuPrime Pfx DNA polymerase.

FIG. 38: Competitive audit of AccuPrime Pfx DNA polymerase against Pfu Turbo DNA Polymerase (Stratagene), Pfu Ultra DNA Polymerase (Stratagene), Tgo DNA Polymerase (Roche), and KOD Hot Start DNA Polymerase (Novagen). Each enzyme was used to amplify targets ranging from 822 bp to 6816 bp using 100 to 200 ng of human genomic DNA (K562, genotyping grade). Those are: 1) c-myc 822 bp; 2) p53 2380 bp; 3) Hbg 3.6 kb; 4) Rhod 6173 bp; and 5) Rhod 6816 bp (see Materials and Methods for detail). The gel shows clear and consistent performance of AccuPrime Pfx DNA polymerase over competitors' products.

FIG. 39: PCR using ThermalAce™ DNA polymerase in conjunction with SSBs. PCR was done using SSBs from M. jannachii, M. thermoautotrophicum or S. solfataricus.

FIG. 40: PCR using ThermalAce™ DNA polymerase in conjunction with SSBs, added individually and in combination.

FIG. 41: Use of Methanococcus jannachii SSB in cycle sequencing with ABI Prism® BigDye™ Terminator Cycle sequencing Kits.

FIG. 42: Use of Methanococcus jannachii SSB in cycle sequencing with ABI Prism® BigDye™ Terminator Cycle sequencing Kits.

FIG. 43: SDS polyacrylamide gel electrophoresis (Novex 4-20% Tris Glycine gel) for expression profiling of recombinant SsoSSB (rSsoSSB; Codon optimized) in various E. coli host: BL21(DE3); BL21(DE3)-AI (arabinose induction); and BL21-CodonPlus (rare codon supplemented). The lysates from bacterial cultures with (lanes 8 to 13) or without (lanes 1 to 6) induction of the SSB protein were heat-treated and loaded on the gel to see the level of protein expressed. In particular, the lanes were loaded as follows: Lanes 1 & 2, duplicate of rSsoSSB from uninduced BL21(DE3)-AI; lanes 3 & 4, duplicate of rSsoSSB from uninduced BL21(DE3); lane 5, wild type SsoSSB from uninduced BL21-CodonPlus; lane 6, wild type SsoSSB from uninduced BL21(DE3)-AI; lanes 8 & 9, duplicate of rSsoSSB from induced BL21(DE3)-AI; lanes 10 & 11, duplicate of rSsoSSB from induced BL21(DE3); lane 12, wild type SsoSSB from induced BL21-CodonPlus; and lane 13, wild type SsoSSB from induced BL21(DE3)-AI. Lane 7 contains purified wild type SsoSSB serving as control.

FIG. 44: Elution profile of EMD-SO₃ column chromatography for rSsoSSB from BL21(DE3) host. Protein eluted after 50-650 mM NaCl gradient, followed by 650 mM NaCl elution. The main protein peak was eluted during the high salt elution Shoulder contains larger amounts of truncated protein. Fractions 26-30 were pooled and dialyzed into storage buffer.

FIG. 45: (A) SDS gel of EMD-SO4 fractions. L is load, FT is load flow through. Fractions 26-30 were pooled. (B) Pooled fractions were dialyzed and 2 or 5 ug were run on SDS gel with the purified Sso SSB from Codon Plus cells. 1 is original from Codon Plus, 2 is rSso SSB from BL21 DE3.

### DETAILED DESCRIPTION OF THE INVENTION

The invention provides methods and materials for nucleic acid synthesis (e.g., PCR-based nucleic acid synthesis). The invention is based, in part, on the surprising discovery that the yield and/or homogeneity of primer extension products made by DNAP can be enhanced by including combinations of anti-DNAP antibodies and/or single strand DNA binding proteins (preferably thermostable SSBs) in nucleic acid synthesis mixtures. Nucleic acid synthesis mixture constituents, nucleic acid synthesis methods, and kits useful for performing the same are described herein, along with a brief glossary of terms commonly used by those skilled in the art of molecular biology.

Nucleic acid. In general, a nucleic acid comprises a contiguous series (a.k.a., “strand” and “sequence”) of nucleotides joined by phosphodiester bonds. A nucleic acid can be single stranded or can be double stranded, where two strands are linked via interstrand interactions between complementary nucleotide bases. A nucleic acid can include naturally occurring nucleotides and/or non-naturally occurring nucleotides (e.g., having non-naturally occurring sugar moieties and/or non-naturally occurring base moieties). A nucleic acid can be ribonucleic acid (RNA, including mRNA) or deoxyribonucleic acid (DNA, including genomic DNA, recombinant DNA, cDNA, and synthetic DNA). A nucleic acid can be a discrete molecule such as a chromosome or a cDNA molecule. A nucleic acid also can be a segment (i.e., a series of nucleotides connected by phosphodiester bonds) of a discrete molecule.

Template. A template is a single stranded nucleic acid that, when a part of a primer-template complex, can serve as a substrate for DNAP or RT. A nucleic acid synthesis mixture can include a single type of template, or can include templates having different nucleotide sequences. By using primers specific for particular templates, primer extension products can be made for a plurality of templates in a nucleic acid synthesis mixture. The plurality of templates can be present within different discrete nucleic acids, or can be present within a discrete nucleic acid.

Templates can be obtained, or can be prepared from nucleic acids present in biological sources (e.g., cells, tissues, organs and organisms). Thus, templates can be obtained, or can be prepared from nucleic acids present in bacteria (e.g., species of Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces), fungi such as yeasts, viruses (e.g., Orthomyxoviridae, Paramyxoviridae, Herpesviridae, Picornaviridiae, Hepadnaviridae, Retroviridiae) protozoa, plants and animals (e.g., insects such as Drosophila spp., nematodes such as Caenorhabditis elegans, fish, birds, rodents, porcines, equines, felines, canines, and primates including humans). Templates also can be obtained, or can be prepared from nucleic acids present in environmental samples such as soil, water and air samples. Nucleic acids can be prepared from such biological and environmental sources using routine methods known by those of skill in the art (see, e.g. Maniatis, T. et al. (1978) Cell 15:687-701; Okayama, H., and P. Berg (1982) Mol. Cell. Biol. 2:161-170; Gubler, U., and B. Hoffman (1983) Gene 25:263-269).

In some embodiments, a template is obtained directly from a biological or environmental source. In other embodiments, a template is provided by wholly or partially denaturing a double-stranded nucleic acid obtained from a biological or environmental source. In some embodiments, a template is a recombinant DNA molecule or a synthetic DNA molecule. Recombinant or synthetic DNA can be single stranded or can be double stranded, in which case it is preferably wholly or partially denatured to provide a template. In some embodiments, a template is an mRNA molecule or population of mRNA molecules. In other embodiments, a template is a cDNA molecule or a population of cDNA molecules. A cDNA template can be synthesized in a nucleic acid synthesis reaction by an enzyme having reverse transcriptase activity, or can be provided from an extrinsic source (e.g., a cDNA library).

Primer. A primer is a single stranded nucleic acid that is shorter than a template, and that is complementary to a segment of a template. A primer can hybridize to a template to form a primer-template complex (i.e., a primed template) such that a DNAP can synthesize a nucleic acid molecule (i.e., primer extension product) that is complementary to all or a portion of a template.

Primers typically are 12 to 60 nucleotides long (e.g., 18 to 45 nucleotides long), although they may be shorter or longer in length. A primer is designed to be substantially complementary to a cognate template such that it can specifically hybridize to the template to form a primer-template complex that can serve as a substrate for DNAP to make a primer extension product. In some primer-template complexes, the primer and template are exactly complementary such that each nucleotide of a primer is complementary to and interacts with a template nucleotide. Primers can be made as a matter of routine by those skilled in the art (e.g., using an ABI DNA Synthesizer from Applied Biosystems or a Biosearch 8600 or 8800 Series Synthesizer from Milligen-Biosearch, Inc.), or can be obtained from a number of commercial vendors.

DNA polymerase (DNAP). A DNA polymerase is an enzyme that can add deoxynucleoside monophosphate molecules to the 3′ hydroxy end of a primer in a primer-template complex, and then sequentially to the 3′ hydroxy end of a growing primer extension product in a template dependent manner (i.e., depending upon the sequence of nucleotides in the template). DNAPs typically add nucleotides that are complementary to the template being used, but DNAPs may add noncomplementary nucleotides (mismatches) during the polymerization or synthesis process. Thus, the synthesized nucleic acid strand may not be completely complementary to the template. DNAPs may also make nucleic acid molecules that are shorter in length than the template used. DNAPs have two preferred substrates: one is the primer-template complex where the primer terminus has a free 3′-hydroxyl group, the other is a deoxynucleotide 5′-triphosphate (dNTP). A phosphodiester bond is formed by nucleophilic attack of the 3′-OH of the primer terminus on the α-phosphate group of the dNTP and elimination of the terminal pyrophosphate. DNAPs can be isolated from organisms as a matter of routine by those skilled in the art, and can be obtained from a number of commercial vendors.

Some DNAPs are thermostable, and are not substantially inactivated at temperatures commonly used in PCR-based nucleic acid synthesis. Such temperatures vary depending upon reaction parameters, including pH, template and primer nucleotide composition, primer length, and salt concentration. Thermostable DNAPs include Thermus thermophilus (Tth) DNAP, Thermus aquaticus (Taq) DNAP, Thermotoga neopolitana (Tne) DNAP, Thermotoga maritima (Tma) DNAP, Thermatoga strain FjSS3-B.1 DNAP, Thermococcus litoralis (Tli or VENT™) DNAP, Pyrococcus furiosus (Pfu) DNAP, DEEPVENT™ DNAP, Pyrococcus woosii (Pwo) DNAP, Pyrococcus sp KOD2 (KOD) DNAP, Bacillus sterothermophilus (Bst) DNAP, Bacillus caldophilus (Bca) DNAP, Sulfolobus acidocaldarius (Sac) DNAP, Thermoplasma acidophilum (Tac) DNAP, Thermus flavus (Tfl/Tub) DNAP, Thermus ruber (Tru) DNAP, Thermus brockianus (DYNAZYME™) DNAP, Thermosipho africanus DNAP, and mutants, variants and derivatives thereof (see e.g., U.S. Pat. No. 6,077,664; U.S. Pat. No. 5,436,149; U.S. Pat. No. 4,889,818; U.S. Pat. No. 5,532,600; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 94/26766; WO 92/06188; WO 92/03556; WO 89/06691; WO 91/09950; 91/09944; WO 92/06200; WO 96/10640; WO 97/09451; Barnes, W. Gene 112:29-35 (1992); Lawyer, F. et al (1993) PCR Meth. Appl. 2:275-287; and Flaman, J. et al. (1994) Nucl. Acids Res. 22:3259-3260).

Other DNAPs are mesophilic, including pol I family DNAPs (e.g., DNAPs from E. coli, H. influenzae, D. radiodurans, H. pylori, C. aurantiacus, R. Prowazekii, T pallidum, Synechocysis sp., B. subtilis, L. lactis, S. pneumoniae, M. tuberculosis, M leprae, M. smegmatis, Bacteriophage L5, phi-C31, T7, T3, T5, SP01, SP02, S. cerevisiae, and D. melanogaster), pol III type DNAPs, and mutants, variants and derivatives thereof.

Reverse Transcriptase (RT). Reverse transcriptases are enzymes having reverse transcriptase activity (i.e., that catalyze synthesis of DNA from a single-stranded RNA template). Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al. (1988) Science 239:487-491; U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see e.g., WO 97/09451 and WO 98/47912). Some RTs have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wild type or RNase H+ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al. (1988) Nucl. Acids Res. 16:265 and in Gerard, G. F., et al. (1992) FOCUS 14:91. Particularly preferred polypeptides for use in the invention include, but are not limited to, M-MLV H⁻ reverse transcriptase, RSV H⁻ reverse transcriptase, AMV H⁻ reverse transcriptase, RAV (rous-associated virus) H⁻ reverse transcriptase, MAV (myeloblastosis-associated virus) H⁻ reverse transcriptase and HIV H⁻ reverse transcriptase (see U.S. Pat. No. 5,244,797 and WO 98/47912). It will be understood by one of skill in the art that any enzyme capable of producing a DNA molecule from a ribonucleic acid molecule (i.e., having reverse transcriptase activity) may be equivalently used in the compositions, methods and kits of the invention.

Nucleotide. A nucleotide consists of a phosphate group linked by a phosphoester bond to a pentose (ribose in RNA, and deoxyribose in DNA) that is linked in turn to an organic base. The monomeric units of a nucleic acid are nucleotides. Naturally occurring DNA and RNA each contain four different nucleotides: nucleotides having adenine, guanine, cytosine and thymine bases are found in naturally occurring DNA, and nucleotides having adenine, guanine, cytosine and uracil bases found in naturally occurring RNA. The bases adenine, guanine, cytosine, thymine, and uracil often are abbreviated A, G, C, T and U, respectively

Nucleotides include free mono-, di- and triphosphate forms (i.e., where the phosphate group has one, two or three phosphate moieties, respectively). Thus, nucleotides include ribonucleoside triphosphates (e.g., ATP, UTP, CTG and GTP) and deoxyribonucleoside triphosphates (e.g., dATP, dCTP, dITP, dGTP and dTTP), and derivatives thereof. Nucleotides also include dideoxyribonucleoside triphosphates (ddNTPs, including ddATP, ddCTP, ddGTP, ddITP and ddTTP), and derivatives thereof.

Nucleotide derivatives include [αS]dATP, 7-deaza-dGTP, 7-deaza-dATP, and nucleotide derivatives that confer resistance to nucleolytic degradation. Nucleotide derivatives include nucleotides that are detectably labeled, e.g., with a radioactive isotope such as ³²P or ³⁵S, a fluorescent moiety, a chemiluminescent moiety, a bioluminescent moiety or an enzyme.

Primer extension product. A primer extension product is a nucleic acid that includes a primer to which DNAP has added one or more nucleotides. Primer extension products can be as long as, or shorter than the template of a primer-template complex.

Amplifying. Amplifying refers to an in vitro method for increasing the number of copies of a nucleic acid with the use of a DNAP. Nucleic acid amplification results in the addition of nucleotides to a primer or growing primer extension product to form a new molecule complementary to a template. In nucleic acid amplification, a primer extension product and its template can be denatured and used as templates to synthesize additional nucleic acid molecules. An amplification reaction can consist of many rounds of replication (e.g., one PCR may consist of 5 to 100 “cycles” of denaturation and primer extension). General methods for amplifying nucleic acids are well-known to those of skill in the art (see e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H., and A. Griffin, eds., PCR Technology: Current Innovations, Boca Raton, Fla.: CRC Press (1994)). Amplification methods that can be used in accord with the present invention include PCR (U.S. Pat. Nos. 4,683,195 and 4,683,202), Strand Displacement Amplification (SDA; U.S. Pat. No. 5,455,166; EP 0 684 315), Nucleic Acid Sequenced-Based Amplification (NASBA; U.S. Pat. No. 5,409,818; EP 0 329 822).

Antibodies. In general, the term “antibody” refers to immunoglobulin molecules (e.g., IgG and IgM molecules) and immunologically active portions of immunoglobulin molecules (e.g., F(ab) and F(ab′)₂ fragments). Single chain antibodies and fragments thereof also are contemplated for use in the invention. Antibodies preferably contain at least one antigen binding site that specifically binds one or more antigens. An anti-DNAP antibody is an antibody that specifically binds to or interacts with a DNAP. An anti-RT antibody is an antibody that specifically binds to or interacts with a RT. Some antibodies are temperature sensitive, specifically binding a cognate antigen at one temperature, and exhibiting reduced antigen-binding at a higher temperature.

Polyclonal antibody preparations include a population of antibody molecules that have different antigen binding sites that can immunoreact with different epitopes (i.e., immunogenic portions) of an antigen (e.g., DNAP or RT). Monoclonal antibody preparations include a population of antibody molecules that have single species of antigen binding site that can immunoreact with a particular epitope of an antigen. A monoclonal antibody composition typically exhibits a single binding affinity for an antigen with which it immunoreacts.

Preferably, anti-DNAP and/or anti-RT antibodies of the invention can be inactivated or substantially inactivated such that they retain less than 25% (e.g., less than 20%, less than 15%, preferably less than 10% and most preferably less than 5%) antigen inhibitory activity compared to a control antibody that has not been subjected to the conditions favoring inactivation. Conditions that can be used to inactivate or substantially inactivate antibodies include, e.g., temperature, pH, ionic conditions, although a change in temperature is preferred. U.S. Pat. No. 5,338,671 discloses temperature sensitive monoclonal IgG anti-DNAP antibodies. Antibodies can be designed or generated to have different temperatures at which the antibody is inactivated or substantially inactivated. Preferably, the temperature at which the antibody is inactivated is greater than 45° C., greater than 50° C., greater than 55° C., greater than 60° C., greater than 65° C., greater than 70° C., greater than 75° C., greater than 80° C., greater than 85° C., greater than 90° C., greater than 95° C. or greater than 100° C.

Anti-DNAP and Anti-RT antibodies can be made by immunizing a suitable subject (e.g., rabbit, goat, mouse or other mammal) with an immunogenic preparation that contains isolated DNAP or RT, or immunogenic portions thereof. An immunogenic preparation can contain, for example, a recombinant DNAP or DNAP portion, or a recombinant RT or RT portion. Immunogenic DNAP or RT portions, including recombinant DNAP or RT portions and portions made by enzymatic or chemical proteolysis, have at least 5 amino acids (e.g., at least 10 amino acids, at least 15 amino acids, at least 20 amino acids, and at least 30 amino acids). Some immunogenic DNAP or RT portions correspond to regions of DNAP or RT that are located on the surface of the enzyme (e.g., hydrophilic regions). An immunogenic preparation also can include an adjuvant, such as Freund's complete or incomplete adjuvant, or other immunostimulatory agent.

Immunizing a suitable subject with an immunogenic DNAP or RT preparation induces a polyclonal anti-DNAP or anti-RT antibody response, respectively. The antibody titer in an immunized subject can be monitored over time using standard techniques (e.g., enzyme linked immunosorbent assay (ELISA)). At an appropriate time after immunization (e.g., when antibody titers are greatest), antibodies can be isolated from the subject (e.g., from the blood) to yield a polyclonal antibody preparation. Antibodies can be further purified using routine techniques (e.g., protein A chromatography to obtain the IgG fraction).

Monoclonal antibodies can be made using standard techniques, such as the hybridoma technique disclosed by Kohler and Milstein (1975) Nature 256:495-497 (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the human B cell hybridoma technique (see e.g., Kozbor et al. (1983) Immunol Today 4:72), the EBV-hybridoma technique (see e.g., Cole et al. (1985), Monoclonal Antibodies and Cancer Therapy, Alan R. Liss, Inc., pp. 77-96), or trioma techniques. Techniques for making monoclonal antibody hybridomas are routine and are well known in the art (see e.g., R. H. Kenneth, in Monoclonal Antibodies: A New Dimension In Biological Analyses, Plenum Publishing Corp., New York, N.Y. (1980); E. A. Lerner (1981) Yale J. Biol. Med. 54:387-402; and M. L. Gefter et al. (1977) Somatic Cell Genet. 3:231-36). Briefly, an immortal cell line (e.g., a myeloma) is fused to lymphocytes (e.g., splenocytes) from a mammal immunized with an immunogen such as DNAP or portion thereof, or RT or portion thereof, and the culture supernatants of the resulting hybridoma cells are screened to identify a hybridoma producing a monoclonal antibody that specifically binds the immunogen.

Monoclonal antibodies can be made using routine protocols for fusing lymphocytes and immortalized cell lines (see e.g., G. Galfre et al. (1977) Nature 266:55052; Gefter et al. Somatic Cell Genet., cited supra; Lerner, Yale J. Biol. Med., cited supra; and Kenneth, Monoclonal Antibodies, cited supra). An immortal cell line (e.g., a myeloma cell line) can be derived from the same mammalian species as the lymphocytes. For example, murine hybridomas can be made by fusing lymphocytes from a mouse immunized with an immunogenic preparation with an immortalized mouse cell line. Immortal cell lines include mouse myeloma cell lines that are sensitive to culture medium containing hypoxanthine, aminopterin and thymidine (“HAT medium”). Exemplary myeloma cell lines that can be used as a fusion partner are the P3-NS1/1-Ag4-1, P3-x63-Ag8.653 or Sp2/O-Ag14 myeloma lines. HAT-sensitive mouse myeloma cells can be fused to mouse splenocytes using polyethylene glycol. Resultant hybridoma cells then can be selected using HAT medium, which kills unfused and unproductively fused myeloma cells. Hybridoma cells producing a monoclonal antibody can be detected by screening the hybridoma culture supernatants for antibodies that specifically bind immunogen, e.g., using an ELISA assay.

Monoclonal anti-DNAP and anti-RT antibodies also can be obtained by screening a recombinant combinatorial immunoglobulin library (e.g., an antibody phage display library) with DNAP or RT or portions thereof to identify library members that bind DNAP or RT. Techniques for making and screening phage display libraries are well known, and kits for accomplishing the same are available commercially. Examples of methods and reagents suitable for making and screening antibody display libraries are disclosed in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Anti-DNAP antibodies suitable for use in the present invention are disclosed, for example in, U.S. Pat. No. 5,338,671. Anti-RT antibodies suitable for use in the present invention are disclosed, for example, in WO0052027A1.

Single stranded DNA binding protein (SSB). Single stranded DNA binding proteins (SSBs) are proteins that preferentially bind single stranded DNA (ssDNA) over double-stranded DNA in a nucleotide sequence independent manner. SSBs have been identified in virtually all known organisms, and appear to be important for DNA metabolism, including replication, recombination and repair. Naturally occurring SSBs typically are comprised of two, three or four subunits, which may be the same or different. In general, naturally occurring SSB subunits contains at least one conserved DNA binding domain, or “OB fold” (see e.g., Philipova, D. et al. (1996) Genes Dev. 10:2222-2233; and Murzin, A. (1993) EMBO J. 12:861-867), such that naturally occurring SSBs have four or more OB folds.

SSBs from mesophilic organisms reportedly can improve PCR efficiency (see e.g., U.S. Pat. Nos. 5,605,824 and 5,773,257; Chou, Q. (1992) Nucl. Acids Res. 20:4371; Rapley, R. (1994) Mol. Biotechnol. 2:295-298; and Dabrowski, S. and J. Kur (1999) Protein Expr Purif 16:96-102). However, the temperatures commonly employed in PCR-based nucleic acid synthesis can exceed the upper limit at which mesophilic SSBs bind DNA, limiting their effectiveness in PCR-based nucleic acid synthesis.

Thermostable SSBs bind ssDNA at 70° C. at least 70% (e.g., at least 80%, at least 85%, at least 90% and at least 95%) as well as they do at 37° C., and are better suited for PCR applications than are mesophilic SSBs. Thermostable SSBs can be obtained from archaea. Archaea are a group of microbes distinguished from eubacteria through 16S rDNA sequence analysis. Archaea can be subdivided into three groups: crenarchaeota, euryarchaeota and korarchaeota (see e.g., Woese, C. and G. Fox (1977) PNAS 74: 5088-5090; Woese, C. et al. (1990) PNAS 87: 4576-4579; and Barns, S. et al. (1996) PNAS 93:9188-9193). Recently, there have been reports on the identification and characterization of euryarchaeota SSBs, including Methanococcus jannachii SSB, Methanobacterium thermoautrophicum SSB, and Archaeoglobus fulgidus SSB, as well as crenarchaeota SSBs, including Sulfolobus sulfataricus SSB and Aeropyrum pernix SSB (see e.g., Chedin, F. et al. (1998) Trends Biochem. Sci. 23:273-277; Haseltine C. et al. (2002) Mol. Microbiol. 43:1505-1515; Kelly, T. et al. (1998) Proc. Natl. Acad. Sci. USA 95:14634-14639; Klenk, H. et al. (1997) Nature 390:364-370; Smith, D. et al. (1997) J. Bacteriol. 179:7135-55; Wadsworth, R. and M. White (2001) Nucl. Acids Res. 29:914-920; and in U.S. Patent Application 60/147,680.

Ordinarily skilled artisans can purify SSBs (including archaea SSBs), make recombinant variants, and can measure SSB activity using routine methods, such as those disclosed in Haseltine C. et al. (2002) Mol. Microbiol. 43:1505-1515.

A non-comprehensive list of known SSBs, with GenBank Accession numbers, is provided in Table 1.

TABLE 1

| | |
|---|---|
| gi\|18978392 | Replication factor A related protein [Pyrococcus furiosus DSM |
| | 3638] |
| gi\|15679384 | Replication factor A related protein [Methanothermobacter |
| | thermautotrophicus] [Methanothermobacter thermautotrophicus str. Delta H] |
| gi\|15679383 | Replication factor A related protein [Methanothermobacter |
| | thermautotrophicus] [Methanothermobacter thermautotrophicus str. Delta H] |
| gi\|15669348 | Replication factor A related protein [Methanococcus jannaschii] |
| | [Methanocaldococcus jannaschii] |
| gi\|14520503 | Replication factor A related protein [Pyrococcus abyssi] |
| gi\|2622495 | Replication factor A related protein [Methanothermobacter |
| | thermautotrophicus str. Delta H] |
| gi\|2622494 | Replication factor A related protein [Methanothermobacter |
| | thermautotrophicus str. Delta H] |
| gi\|18894230 | Replication factor A related protein [Pyrococcus furiosus DSM |
| | 3638] |
| gi\|7521609 | Replication factor A related protein PAB2163 - Pyrococcus abyssi |
| | (strain Orsay) |
| gi\|7482812 | Replication factor A related protein - Methanobacterium |
| | thermoautotrophicum (strain Delta H) |
| gi\|7482811 | Replication factor A related protein - Methanobacterium |
| | thermoautotrophicum (strain Delta H) |
| gi\|5457718 | Replication factor A related protein [Pyrococcus abyssi] |
| gi\|1500014 | Replication factor A related protein [Methanococcus jannaschii] |
| | [Methanocaldococcus jannaschii] |
| gi\|22299033 | Single-stranded DNA-binding protein [Thermosynechococcus |
| | elongatus BP-1] |
| gi\|17545141 | Single-strand Binding Protein (Helix Destabilizing Protein) |
| | [Ralstonia solanacearum] |
| gi\|15807618 | Single-stranded DNA-binding protein [Deinococcus |
| | radiodurans] |
| gi\|15645859 | Single-strand DNA-binding protein (ssb) [Helicobacter pylori |
| | 26695] |
| gi\|15616611 | Single-strand DNA-binding protein (phage-related protein) |
| | [Bacillus halodurans] |
| gi\|21233884 | Single-strand DNA binding protein [Proteus vulgaris] |
| gi\|21233779 | Single-strand DNA binding protein [Proteus vulgaris] |
| gi\|21233694 | Single-strand DNA binding protein [Proteus vulgaris] |
| gi\|21203068 | Single-strand DNA binding protein [Proteus vulgaris] |
| gi\|21202963 | Single-strand DNA binding protein [Proteus vulgaris] |
| gi\|21202878 | Single-strand DNA binding protein [Proteus vulgaris] |
| gi\|6767506 | ssDNA-binding protein controls activity of RecBCD nuclease |
| | [Salmonella typhimurium LT2] |
| gi\|19746763 | Single strand binding protein [Streptococcus pyogenes |
| | MGAS8232] |
| gi\|19746681 | Single strand binding protein [Streptococcus pyogenes |
| | MGAS8232] |
| gi\|19745475 | Single strand binding protein [Streptococcus pyogenes |
| | MGAS8232] |
| gi\|19745296 | Single-strand binding protein [Streptococcus pyogenes |
| | MGAS8232] |
| gi\|22295215 | Single-stranded DNA-binding protein [Thermosynechococcus |
| | elongatus BP-1] |
| gi\|21325755 | Single-stranded DNA-binding protein [Corynebacterium |
| | glutamicum ATCC 13032] |
| gi\|21324632 | Single-stranded DNA-binding protein [Corynebacterium |
| | glutamicum ATCC 13032] |
| gi\|205544 | Single-stranded DNA binding protein precursor [Rattus sp.] |
| gi\|22124496 | ssDNA-binding protein [Yersinia pestis KIM] |
| gi\|586039 | Single-strand binding protein (SSB) (Helix-destabilizing protein) |
| gi\|417811 | Single-stranded DNA-binding protein, mitochondrial precursor |
| | (Mt-SSB) (MtSSB) (PWP1-interacting protein 17) |
| gi\|17137188 | Clp-P1; Single stranded-binding protein c6a [Drosophila |
| | melanogaster] |
| gi\|17137156 | Ssb-c31a-P1 [Drosophila melanogaster] |
| gi\|16422814 | ssDNA-binding protein [Salmonella typhimurium LT2] |
| gi\|21957289 | ssDNA-binding protein [Yersinia pestis KIM] |
| gi\|18249854 | Single-stranded DNA binding protein [Aster yellows |
| | phytoplasma] |
| gi\|17981729 | Mitochondrial single stranded DNA-binding protein; low power |
| | [Drosophila melanogaster] |
| gi\|10955315 | Single-strand binding protein [Escherichia coli] |
| gi\|9507481 | Single-stranded DNA binding protein [Plasmid ColIb-P9] |
| gi\|21911117 | Single strand DNA binding protein [Streptococcus pyogenes |
| | MGAS315] |
| gi\|21905327 | Single strand DNA binding protein [Streptococcus pyogenes |
| | MGAS315] |
| gi\|21885285 | Single-stranded DNA binding protein [Vibrio cholerae] |
| gi\|16751957 | Single-stranded DNA binding protein [Plasmid pIPO2T] |
| gi\|16610025 | Single-stranded DNA binding protein [Plasmid pIPO2T] |
| gi\|6968505 | Single-strand DNA binding protein [Campylobacter jejuni subsp. |
| | jejuni NCTC 11168] |
| gi\|18146307 | Phage-related single-strand DNA-binding protein [Clostridium |
| | perfringens str. 13] |
| gi\|18143945 | Phage-related single-strand DNA binding protein [Clostridium |
| | perfringens str. 13] |
| gi\|9626285 | Single-stranded DNA binding protein [Bacteriophage lambda] |
| gi\|21686516 | Single-stranded DNA-binding protein [Arthrobacter aurescens] |
| gi\|21672790 | Single-strand binding protein [Buchnera aphidicola str. Sg |
| | (Schizaphis graminum)] |
| gi\|21203507 | Single-strand DNA-binding protein of phage phi Sa 2mw |
| | [Staphylococcus aureus subsp. aureus MW2] |
| gi\|13700280 | Single-strand DNA-binding protein of phage phi PVL |
| | [Staphylococcus aureus subsp. aureus N315] |
| gi\|21628947 | Single-strand DNA binding (helix-destabilizing) protein |
| | [Haemophilus influenzae biotype aegyptius] |
| gi\|21623439 | Single-strand binding protein [Buchnera aphidicola str. Sg |
| | (Schizaphis graminum)] |
| gi\|21243632 | Single-stranded DNA binding protein [Xanthomonas axonopodis |
| | pv. citri str. 306] |
| gi\|21242946 | Single-stranded DNA binding protein [Xanthomonas axonopodis |
| | pv. citri str. 306] |
| gi\|20809109 | Single-stranded DNA-binding protein [Thermoanaerobacter |
| | tengcongensis] |
| gi\|20808452 | Single-stranded DNA-binding protein [Thermoanaerobacter |
| | tengcongensis] |
| gi\|20807311 | Single-stranded DNA-binding protein [Thermoanaerobacter |
| | tengcongensis] |
| gi\|21591574 | Single-strand DNA binding (helix-destabilizing) protein |
| | [Haemophilus influenzae biotype aegyptius] |
| gi\|17935411 | Single-strand DNA binding protein [Agrobacterium tumefaciens |
| | str. C58] |
| gi\|16272208 | Single-stranded DNA binding protein (ssb) [Haemophilus |
| | influenzae Rd] |
| gi\|16131885 | ssDNA-binding protein [Escherichia coli K12] |
| gi\|15834295 | ssDNA-binding protein [Escherichia coli O157:H7] |
| gi\|15804651 | ssDNA-binding protein [Escherichia coli O157:H7 EDL933] |
| gi\|18311623 | Phage-related single-strand DNA-binding protein [Clostridium |
| | perfringens] |
| gi\|18309269 | Phage-related single-strand DNA binding protein [Clostridium |
| | perfringens] |
| gi\|16802093 | Single-strand binding protein (SSB) [Listeria monocytogenes |
| | EGD-e] |
| gi\|16799117 | Single-strand binding protein (SSB) [Listeria innocua] |
| gi\|16763010 | Single strand binding protein [Salmonella enterica subsp. |
| | enterica serovar Typhi] |
| gi\|16762936 | Single-strand DNA-binding protein [Salmonella enterica subsp. |
| | enterica serovar Typhi] |
| gi\|16332050 | Single-stranded DNA-binding protein [Synechocystis sp. PCC |
| | 6803] |
| gi\|16120662 | Single-strand binding protein [Yersinia pestis] |
| gi\|16081142 | Single-strand DNA-binding protein [Bacillus subtilis] |
| gi\|15965311 | Single-strand binding protein [Sinorhizobium meliloti] |
| gi\|15926067 | Single-strand DNA-binding protein of phage phi PVL |
| | [Staphylococcus aureus subsp. aureus N315] |
| gi\|15923356 | Single-strand DNA-binding protein of phage phi PVL |
| | [Staphylococcus aureus subsp. aureus Mu50] |
| gi\|15899120 | Single-stranded DNA binding protein (SSB) [Sulfolobus |
| | solfataricus] |
| gi\|15896954 | Single strand DNA binding protein, SSB [Clostridium |
| | acetobutylicum] |
| gi\|15895648 | Single-strand DNA-binding protein, ssb [Clostridium |
| | acetobutylicum] |
| gi\|15895193 | Phage related SSB-like protein [Clostridium acetobutylicum] |
| gi\|15894232 | Single-stranded DNA-binding protein [Clostridium |
| | acetobutylicum] |
| gi\|15893218 | Single-strand binding protein [Rickettsia conorii] |
| gi\|15835919 | SS DNA binding protein [Chlamydophila pneumoniae J138] |
| gi\|15829081 | Single-strand DNA binding protein (SSB) (Helix destabilizing |
| | protein) [Mycoplasma pulmonis] |
| gi\|15828449 | Single strand binding protein [Mycobacterium leprae] |
| gi\|15794566 | Single-strand binding protein [Neisseria meningitidis Z2491] |
| gi\|15792396 | Single-strand binding protein [Campylobacter jejuni] |
| gi\|15618301 | SS DNA Binding Protein [Chlamydophila pneumoniae |
| | CWL029] |
| gi\|15617138 | Single-strand binding protein [Buchnera sp. APS] |
| gi\|15612231 | Single-strand binding protein [Helicobacter pylori J99] |
| gi\|15607196 | ssb [Mycobacterium tuberculosis H37Rv] |
| gi\|15605660 | Single stranded DNA-binding protein [Aquifex aeolicus] |
| gi\|15604763 | SS DNA Binding Protein [Chlamydia trachomatis] |
| gi\|15604667 | Single-strand binding protein (ssb) [Rickettsia prowazekii] |
| gi\|15603815 | Ssb [Pasteurella multocida] |
| gi\|15599428 | Single-stranded DNA-binding protein [Pseudomonas |
| | aeruginosa] |
| gi\|13507968 | Single-stranded DNA binding protein [Mycoplasma |
| | pneumoniae] |
| gi\|13358117 | Single-strand binding protein [Ureaplasma urealyticum] |
| gi\|12044943 | Single-stranded DNA-binding protein (ssb) [Mycoplasma |
| | genitalium] |
| gi\|21539818 | Ssb [Lactococcus lactis subsp. cremoris] |
| gi\|15639056 | Single-strand DNA binding protein (ssb) [Treponema pallidum] |
| gi\|15594460 | Single-stranded DNA-binding protein (ssb) [Borrelia |
| | burgdorferi] |
| gi\|17865707 | Single-strand binding protein (SSB) (Helix-destabilizing protein) |
| gi\|8478517 | Single-strand binding protein (SSB) (Helix-destabilizing protein) |
| gi\|1174443 | Single-strand binding protein (SSB) (Helix-destabilizing protein) |
| gi\|417647 | Single-stranded DNA-binding protein RIM1, mitochondrial |
| | precursor (ssDNA-binding protein, mitochondrial) |
| gi\|138390 | Single-stranded DNA binding protein (Helix-destabilizing protein) |
| | (Gp32) |
| gi\|134913 | Single-strand binding protein (SSB) (Helix-destabilizing protein) |
| gi\|21400036 | SSB, Single-strand binding protein family [Bacillus anthracis |
| | A2012] [Bacillus anthracis str. A2012] |
| gi\|21397955 | SSB, Single-strand binding protein family [Bacillus anthracis |
| | A2012] [Bacillus anthracis str. A2012] |
| gi\|18920500 | ssb [Staphylococcus aureus phage phi 11] |
| gi\|16505317 | Single strand binding protein [Salmonella enterica subsp. |
| | enterica serovar Typhi] |
| gi\|16505243 | Single-strand DNA-binding protein [Salmonella enterica subsp. |
| | enterica serovar Typhi] |
| gi\|16412459 | Single-strand binding protein (SSB) [Listeria innocua] |
| gi\|16409404 | Single-strand binding protein (SSB) [Listeria monocytogenes] |
| gi\|15978425 | Single-strand binding protein [Yersinia pestis] |
| gi\|21232166 | Single-stranded DNA binding protein [Xanthomonas campestris |
| | pv. campestris str. ATCC 33913] |
| gi\|21282071 | Single-strand DNA-binding protein of phage phi Sa 2 mw |
| | [Staphylococcus aureus subsp. aureus MW2] |
| gi\|21222314 | Single-strand DNA-binding protein [Streptomyces coelicolor |
| | A3(2)] |
| gi\|21221138 | Single-strand DNA-binding protein [Streptomyces coelicolor |
| | A3(2)] |
| gi\|21109208 | Single-stranded DNA binding protein [Xanthomonas axonopodis |
| | pv. citri str. 306] |
| gi\|21108448 | Single-stranded DNA binding protein [Xanthomonas axonopodis |
| | pv. citri str. 306] |
| gi\|8978758 | SS DNA binding protein [Chlamydophila pneumoniae J138] |
| gi\|21113919 | Single-stranded DNA binding protein [Xanthomonas campestris |
| | pv. campestris str. ATCC 33913] |
| gi\|20910891 | Single-stranded DNA binding protein, mitochondrial precursor |
| | (MT-SSB) (MTSSB) (P16) [Mus musculus] |
| gi\|8052392 | Single-strand DNA-binding protein [Streptomyces coelicolor |
| | A3(2)] |
| gi\|4808403 | Single-strand DNA-binding protein [Streptomyces coelicolor |
| | A3(2)] |
| gi\|20517787 | Single-stranded DNA-binding protein [Thermoanaerobacter |
| | tengcongensis] |
| gi\|20517069 | Single-stranded DNA-binding protein [Thermoanaerobacter |
| | tengcongensis] |
| gi\|20515823 | Single-stranded DNA-binding protein [Thermoanaerobacter |
| | tengcongensis] |
| gi\|15074901 | SSB protein [Streptococcus pneumoniae bacteriophage MM1] |
| gi\|19748994 | Single strand binding protein [Streptococcus pyogenes |
| | MGAS8232] |
| gi\|19748904 | Single strand binding protein [Streptococcus pyogenes |
| | MGAS8232] |
| gi\|19747591 | Single strand binding protein [Streptococcus pyogenes |
| | MGAS8232] |
| gi\|19747395 | Single-strand binding protein [Streptococcus pyogenes |
| | MGAS8232] |
| gi\|6647829 | Single-strand binding protein (SSB) (Helix-destabilizing protein) |
| gi\|13432209 | Single-strand binding protein (SSB) (Helix-destabilizing protein) |
| gi\|1711533 | Single-stranded DNA-binding protein, mitochondrial precursor |
| | (Mt-SSB) (MtSSB) |
| gi\|10956609 | Single-strand binding protein homolog Ssb [Corynebacterium |
| | glutamicum] |
| gi\|19352383 | Ssb protein [uncultured bacterium] |
| gi\|15088755 | SSB protein [Streptococcus pneumoniae bacteriophage MM1] |
| gi\|19070050 | Ssb protein [uncultured bacterium] |
| gi\|19032310 | Single-stranded DNA-binding protein [Anabaena variabilis] |
| gi\|18920719 | Single-strand binding protein Ssb [Bartonella bacilliformis] |
| gi\|11875133 | Single-stranded DNA binding protein [Escherichia coli |
| | O157:H7] |
| gi\|8918883 | Single-strand DNA binding protein [Plasmid F] |
| gi\|7649839 | Ea10 protein; Ssb [Escherichia coli O157:H7] |
| gi\|5103190 | Single strand DNA binding protein [Plasmid R100] |
| gi\|15919964 | Ssb protein [Plasmid pSB102] |
| gi\|15722263 | Ssb protein [Plasmid pSB102] |
| gi\|18654211 | Single strand binding protein [Bacteriophage LL-H] |
| gi\|14246134 | Single-strand DNA-binding protein of phage phi PVL |
| | [Staphylococcus aureus subsp. aureus Mu50] |
| gi\|14195223 | Single-strand binding protein (SSB) (Helix-destabilizing protein) |
| gi\|11387162 | Single-strand binding protein (SSB) (Helix-destabilizing protein) |
| gi\|6647831 | Single-strand binding protein (SSB) (Helix-destabilizing protein) |
| gi\|11387134 | Single-strand binding protein (SSB) (Helix-destabilizing protein) |
| gi\|6647828 | Single-strand binding protein (SSB) (Helix-destabilizing protein) |
| gi\|6647827 | Single-strand binding protein (SSB) (Helix-destabilizing protein) |
| gi\|6647825 | Single-strand binding protein (SSB) (Helix-destabilizing protein) |
| gi\|6647824 | Single-strand binding protein (SSB) (Helix-destabilizing protein) |
| gi\|6647823 | Single-strand binding protein (SSB) (Helix-destabilizing protein) |
| gi\|6647820 | Single-strand binding protein (SSB) (Helix-destabilizing protein) |
| gi\|6647819 | Single-strand binding protein (SSB) (Helix-destabilizing protein) |
| gi\|2500889 | Single-stranded DNA binding protein |
| gi\|1351118 | Single-stranded DNA binding protein |
| gi\|730833 | Single-strand binding protein (SSB) (Helix-destabilizing protein) |
| gi\|134905 | Single-strand binding protein (SSB) (Helix-destabilizing protein) |
| gi\|4507231 | Single-stranded DNA-binding protein 1 [Homo sapiens] |
| gi\|14794570 | Ssb [Cloning vector pRK310] |
| gi\|18150888 | SSB protein [Pseudomonas putida] |
| gi\|18143627 | Single-stranded DNA binding protein [Aster yellows |
| | phytoplasma] |
| gi\|18104278 | ssb protein [Enterococcus faecalis] |
| gi\|18104262 | ssb protein [Enterococcus faecalis] |
| gi\|18077129 | SSB protein [Pseudomonas putida] |
| gi\|17977995 | Single stranded DNA-binding protein SSB [Escherichia coli] |
| gi\|17864928 | SSB-like protein [Haemophilus influenzae biotype aegyptius] |
| gi\|17739937 | Single-strand DNA binding protein [Agrobacterium tumefaciens |
| | str. C58] |
| gi\|9507773 | Single-strand DNA binding protein [Plasmid F] |
| gi\|9507591 | Single strand DNA binding protein [Plasmid R100] |
| gi\|17427432 | Single-strand binding protein (helix destabilizing protein) |
| | [Ralstonia solanacearum] |
| gi\|17381298 | SSB protein [uncultured bacterium] |
| gi\|13561952 | Single-stranded DNA-binding protein [Mycobacterium |
| | smegmatis] |
| gi\|12830947 | SSB [bacteriophage bIL286] |
| gi\|12830884 | SSB protein [bacteriophage bIL285] |
| gi\|5001700 | Single-strand binding protein; SSB [Bacteriophage Tuc2009] |
| gi\|82212 | ssb protein homolog -common tobacco chloroplast |
| gi\|13786543 | SSB [Lactococcus lactis bacteriophage TP901-1] |
| gi\|13661686 | SSB [Lactococcus lactis bacteriophage TP901-1] |
| gi\|13095695 | SSB protein [bacteriophage bIL285] |
| gi\|12829834 | Single stranded binding protein [Lactococcus lactis |
| | bacteriophage TP901-1] |
| gi\|12248112 | SSB [Bacillus phage GA-1] |
| gi\|9632484 | Single-stranded DNA binding protein [Bacteriophage 933W] |
| gi\|16973267 | ssb protein [uncultured bacterium] |
| gi\|16798847 | SSB protein [Bacteriophage A118] |
| gi\|13487814 | Single-strand binding protein; SSB [Bacteriophage Tuc2009] |
| gi\|13095758 | SSB [bacteriophage bIL286] |
| gi\|12141282 | SSB [Bacillus phage GA-1] |
| gi\|7960759 | Single-stranded DNA binding protein [Bacillus phage Nf] |
| gi\|6094357 | Single-strand binding protein (SSB) (EARLY PROTEIN GP5) |
| gi\|6094356 | Single-strand binding protein (SSB) (EARLY PROTEIN GP5) |
| gi\|5823662 | SSB protein [Bacteriophage A118] |
| gi\|5354247 | ssb; helix-destabilizing [Enterobacteria phage T4] |
| gi\|4426959 | Single-stranded DNA-binding protein SSB-P1 [Enterobacteria |
| | phage P1] |
| gi\|4262664 | SSB [Bacteriophage TuIb] |
| gi\|4262663 | SSB [Bacteriophage Mi] |
| gi\|3915274 | Single-stranded DNA binding protein (helix destabilizing protein) |
| | (GP32) |
| gi\|3915273 | Single-stranded DNA binding protein (helix destabilizing protein) |
| | (GP32) |
| gi\|3915272 | Single-stranded DNA binding protein (helix destabilizing protein) |
| | (GP32) |
| gi\|3915271 | Single-stranded DNA binding protein (helix destabilizing protein) |
| | (GP32) |
| gi\|3915270 | Single-stranded DNA binding protein (helix destabilizing protein) |
| | (GP32) |
| gi\|3915269 | Single-stranded DNA binding protein (helix destabilizing protein) |
| | (GP32) |
| gi\|3915268 | Single-stranded DNA binding protein (helix destabilizing protein) |
| | (GP32) |
| gi\|3915267 | Single-stranded DNA binding protein (helix destabilizing protein) |
| | (GP32) |
| gi\|3915266 | Single-stranded DNA binding protein (helix destabilizing protein) |
| | (GP32) |
| gi\|3915265 | Single-stranded DNA binding protein (helix destabilizing protein) |
| | (GP32) |
| gi\|3915264 | Single-stranded DNA binding protein (helix destabilizing protein) |
| | (GP32) |
| gi\|3915263 | Single-stranded DNA binding protein (helix destabilizing protein) |
| | (GP32) |
| gi\|3915262 | Single-stranded DNA binding protein (helix destabilizing protein) |
| | (GP32) |
| gi\|3915261 | Single-stranded DNA binding protein (helix destabilizing protein) |
| | (GP32) |
| gi\|3915248 | Single-stranded DNA binding protein (helix destabilizing protein) |
| | (GP32) |
| gi\|3915242 | Single-stranded DNA binding protein (helix destabilizing protein) |
| | (GP32) |
| gi\|2645797 | SSB [Bacteriophage SV76] |
| gi\|2645795 | SSB [Bacteriophage RB69] |
| gi\|2645793 | SSB [Bacteriophage RB32] |
| gi\|2645791 | SSB [Bacteriophage RB27] |
| gi\|2645789 | SSB [Bacteriophage RB18] |
| gi\|2645787 | SSB [Bacteriophage RB15] |
| gi\|2645785 | SSB [Bacteriophage RB10] |
| gi\|2645783 | SSB [Bacteriophage RB9] |
| gi\|2645781 | SSB [Bacteriophage RB8] |
| gi\|2645779 | SSB [Bacteriophage RB6] |
| gi\|2645777 | SSB [Bacteriophage RB3] |
| gi\|2645775 | SSB [Bacteriophage PST] |
| gi\|2645773 | SSB [Bacteriophage M1] |
| gi\|2645770 | SSB [bacteriophage FS-alpha] |
| gi\|2645768 | SSB [Enterobacteria phage SV14] |
| gi\|2645766 | SSB [Bacteriophage RB70] |
| gi\|1429233 | SSB [Bacteriophage B103] |
| gi\|138392 | Helix-destabilizing protein (Single-stranded DNA-binding protein) |
| | (SSB protein) |
| gi\|138391 | Single-stranded DNA binding protein (Helix-destabilizing protein) |
| | (GP32) |
| gi\|138389 | Helix-destabilizing protein (Single-stranded DNA-binding protein) |
| | (SSB protein) |
| gi\|138388 | Single-stranded DNA binding protein (Helix-destabilizing protein) |
| | (GP32) |
| gi\|138072 | Single-strand binding protein (SSB) (Early protein GP5) |
| gi\|13937510 | SSB protein [Pseudomonas sp. ADP] |
| gi\|15620434 | Single-strand binding protein [Rickettsia conorii] |
| gi\|1568593 | ssb [Mycobacterium tuberculosis H37Rv] |
| gi\|10955209 | SSB [Enterobacter aerogenes] |
| gi\|1572546 | SSB [Enterobacter aerogenes] |
| gi\|15026829 | Single strand DNA binding protein, SSB [Clostridium |
| | acetobutylicum] |
| gi\|15025394 | Single-strand DNA-binding protein, ssb [Clostridium |
| | acetobutylicum] |
| gi\|15024899 | Phage related SSB-like protein [Clostridium acetobutylicum] |
| gi\|13815667 | Single-stranded DNA binding protein (SSB) [Sulfolobus |
| | solfataricus] |
| gi\|9837391 | Ssb [Flavobacterium johnsoniae] |
| gi\|14090025 | Single-strand binding protein (SSB) (Helix-destabilizing protein) |
| | [Mycoplasma pulmonis] |
| gi\|13992542 | Single-stranded DNA binding [Oryctolagus cuniculus] |
| gi\|13774090 | Single-stranded DNA binding protein [Aster yellows |
| | phytoplasma] |
| gi\|13661656 | Single strand binding protein Ssb [Comamonas testosteroni] |
| gi\|12519013 | ssDNA-binding protein [Escherichia coli O157:H7 EDL933] |
| gi\|13364518 | ssDNA-binding protein [Escherichia coli O157:H7] |
| gi\|4115492 | Single strand binding protein [Phytoplasma sp.] |
| gi\|12722386 | Ssb [Pasteurella multocida] |
| gi\|10954410 | Single strand binding protein [Actinobacillus |
| | actinomycetemcomitans] |
| gi\|10880887 | Single strand binding protein [Actinobacillus |
| | actinomycetemcomitans] |
| gi\|13093879 | Single strand binding protein [Mycobacterium leprae] |
| gi\|4583407 | Single-strand binding protein homolog Ssb [Corynebacterium |
| | glutamicum] |
| gi\|10176674 | Single-strand DNA-binding protein (phage-related protein) |
| | [Bacillus halodurans] |
| gi\|7380314 | Single-stranded binding protein [Neisseria meningitidis Z2491] |
| gi\|4376665 | SS DNA Binding Protein [Chlamydophila pneumoniae CWL029] |
| gi\|1790494 | ssDNA-binding protein [Escherichia coli K12] |
| gi\|7428645 | Single-stranded DNA-binding protein 1 precursor, mitochondrial - |
| | African clawed frog |
| gi\|1674304 | Single-stranded DNA binding protein [Mycoplasma pneumoniae] |
| gi\|7439948 | Single-strand binding protein (ssb) RP836 -Rickettsia prowazekii |
| gi\|7439930 | ssb protein - Mycobacterium tuberculosis (strain H37RV) |
| gi\|7439921 | Single-stranded DNA-binding protein 2 precursor, mitochondrial - |
| | African clawed frog |
| gi\|2146650 | Single-stranded DNA-binding protein ssb - Mycoplasma |
| | pneumoniae (strain ATCC 29342) |
| gi\|2127217 | Single-stranded DNA-binding protein ssb - Bacillus subtilis |
| gi\|2120579 | Single-stranded DNA-binding protein - Brucella abortus |
| gi\|2119790 | Excinuclease ABC chain A - Brucella abortus (fragment) |
| gi\|423723 | Single-stranded mitochondrial DNA-binding protein precursor - rat |
| gi\|423082 | Single-stranded mitochondrial DNA-binding protein precursor - |
| | human |
| gi\|96089 | Helix-destabilizing protein - plasmid RK2 |
| gi\|3328436 | SS DNA Binding Protein [Chlamydia trachomatis] |
| gi\|6899559 | Single-strand binding protein [Ureaplasma urealyticum] |
| gi\|7297359 | Ssb-c31a gene product [Drosophila melanogaster] |
| gi\|9954966 | Chain D, Crystal Structure Of Chymotryptic Fragment Of E. Coli |
| | Ssb Bound To Two 35-Mer Single Strand Dnas |
| gi\|9954965 | Chain C, Crystal Structure Of Chymotryptic Fragment Of E. Coli |
| | Ssb Bound To Two 35-Mer Single Strand Dnas |
| gi\|9954964 | Chain B, Crystal Structure Of Chymotryptic Fragment Of E. Coli |
| | Ssb Bound To Two 35-Mer Single Strand Dnas |
| gi\|9954963 | Chain A, Crystal Structure Of Chymotryptic Fragment Of E. Coli |
| | Ssb Bound To Two 35-Mer Single Strand Dnas |
| gi\|10039203 | Single-strand binding protein [Buchnera sp. APS] |
| gi\|9950448 | Single-stranded DNA-binding protein [Pseudomonas aeruginosa] |
| gi\|9230773 | Single stranded DNA-binding protein [Thermus aquaticus] |
| gi\|6841054 | Single-stranded DNA-binding protein [Borrelia hermsii] |
| gi\|8569292 | Chain D, Crystal Structure Analysis Of Single Stranded Dna |
| | Binding Protein (Ssb) From E. Coli |
| gi\|8569291 | Chain C, Crystal Structure Analysis Of Single Stranded Dna |
| | Binding Protein (Ssb) From E. Coli |
| gi\|8569290 | Chain B, Crystal Structure Analysis Of Single Stranded Dna |
| | Binding Protein (Ssb) From E. Coli |
| gi\|8569289 | Chain A, Crystal Structure Analysis Of Single Stranded Dna |
| | Binding Protein (Ssb) From E. Coli |
| gi\|8548923 | Single stranded binding protein [Thermus thermophilus] |
| gi\|7388261 | Single-strand binding protein (SSB) (Helix destabilizing protein) |
| gi\|2815500 | Single-strand DNA-binding protein R, mitochondrial precursor |
| | (MT-SSB-R) (MT-SSB 2) |
| gi\|586040 | Single-strand binding protein (SSB) (Helix destabilizing protein) |
| gi\|417812 | Single-strand DNA-binding protein, mitochondrial precursor (MT- |
| | SSB) (MTSSB) (P16) |
| gi\|134916 | Single-strand binding protein (SSB) (Helix destabilizing protein) |
| gi\|134914 | Single-strand binding protein (SSB) (Helix destabilizing protein) |
| gi\|134912 | Single-strand DNA-binding protein S, mitochondrial precursor |
| | (MT-SSB-S) (MT-SSB 1) |
| gi\|134910 | Single-strand binding protein (SSB) (Helix destabilizing protein) |
| gi\|134906 | Single-strand binding protein (SSB) (Helix destabilizing protein) |
| gi\|134904 | Single-strand binding protein (SSB) (Helix destabilizing protein) |
| gi\|134903 | Single-strand binding protein (SSB) (Helix destabilizing protein) |
| gi\|6513859 | Single strand binding protein [Salmonella typhi] |
| gi\|7439942 | Single-stranded DNA-binding protein (ssb) homolog - Lyme |
| | disease spirochete |
| gi\|7439928 | Single-strand DNA binding protein (ssb) - syphilis spirochete |
| gi\|7428646 | Single-stranded DNA-binding protein - Escherichia coli |
| gi\|1361850 | Single-stranded DNA binding protein ssb homolog - Mycoplasma |
| | genitalium |
| gi\|484396 | Single-stranded DNA-binding protein - Serratia marcescens |
| gi\|70818 | Single-stranded DNA-binding protein - Escherichia coli plasmid F |
| gi\|70817 | Single-stranded DNA-binding protein - Escherichia coli plasmid |
| | ColIb-P9 |
| gi\|7264824 | Single-stranded DNA-binding protein [Escherichia coli] |
| gi\|3114758 | Single strand DNA binding protein [Campylobacter jejuni] |
| gi\|6739548 | SSB protein [Thermus thermophilus] |
| gi\|466378 | SSB [Plasmid R751] |
| gi\|2735512 | SSB [Staphylococcus carnosus] |
| gi\|4688844 | SSB protein [Escherichia coli] |
| gi\|6066193 | Single strand binding protein [Sinorhizobium meliloti] |
| gi\|6015512 | SSB-like protein [unidentified] |
| gi\|2959411 | Single-stranded binding protein [Mycobacterium leprae] |
| gi\|5702178 | Single stranded DNA binding protein [Escherichia coli] |
| gi\|3337047 | Single-strand binding protein [Escherichia coli] |
| gi\|4585395 | Single-stranded DNA binding protein [Bacteriophage 933W] |
| gi\|2314411 | Single-strand DNA-binding protein (ssb) [Helicobacter pylori |
| | 26695] |
| gi\|4512478 | Single-stranded DNA binding protein [Plasmid ColIb-P9] |
| gi\|4377534 | ssb protein [Escherichia coli] |
| gi\|3851548 | Single strand DNA-binding protein; SSB [Vibrio cholerae] |
| gi\|4261534 | Single-stranded DNA binding protein [Saccharomyces cerevisiae] |
| gi\|4155774 | Single-strand binding protein [Helicobacter pylori J99] |
| gi\|4099056 | Single-stranded DNA binding protein [Rhodobacter sphaeroides] |
| gi\|3861362 | Single-strand binding protein (ssb) [Rickettsia prowazekii] |
| gi\|3844678 | Single-stranded DNA-binding protein (ssb) [Mycoplasma |
| | genitalium] |
| gi\|3822198 | Single strand binding protein [Escherichia coli O157:H7] |
| gi\|2780888 | Chain D, Structure Of Single Stranded Dna Binding Protein (Ssb) |
| gi\|2780887 | Chain C, Structure Of Single Stranded Dna Binding Protein (Ssb) |
| gi\|2780886 | Chain B, Structure Of Single Stranded Dna Binding Protein (Ssb) |
| gi\|2780885 | Chain A, Structure Of Single Stranded Dna Binding Protein (Ssb) |
| gi\|2687989 | Single-stranded DNA-binding protein (ssb) [Borrelia burgdorferi] |
| gi\|3322320 | Single-strand DNA binding protein (ssb) [Treponema pallidum] |
| gi\|1502417 | Single-stranded DNA binding protein p12 subunit |
| | [Schizosaccharomyces pombe] |
| gi\|1502415 | Single-stranded DNA binding protein p30 subunit |
| | [Schizosaccharomyces pombe] |
| gi\|1502413 | Single-stranded DNA binding protein p68 subunit |
| | [Schizosaccharomyces pombe] |
| gi\|396394 | Single-strand DNA-binding protein [Escherichia coli] |
| gi\|3600051 | Similar to the single-strand binding proteins family (Pfam: |
| | SSB.hmm, score: 24.02) [Arabidopsis thaliana] |
| gi\|3323586 | Single-strand binding protein [Salmonella typhimurium] |
| gi\|1573216 | Single-stranded DNA binding protein (ssb) [Haemophilus |
| | influenzae Rd] |
| gi\|2982816 | Single stranded DNA-binding protein [Aquifex aeolicus] |
| gi\|2636637 | Single-strand DNA-binding protein [Bacillus subtilis] |
| gi\|467374 | Single strand DNA binding protein [Bacillus subtilis] |
| gi\|104268 | Single-stranded DNA-binding protein r - African clawed frog |
| | mitochondrion (SGC1) |
| gi\|104182 | Single-stranded DNA-binding protein 1 precursor, mitochondrial - |
| | African clawed frog |
| gi\|1490785 | Single stranded DNA-binding protein [Shewanella sp. SC2A] |
| gi\|1490783 | Single stranded DNA-binding protein [Shewanella sp. F1A] |
| gi\|1490781 | Single stranded DNA-binding protein [Shewanella sp. PT99] |
| gi\|1490779 | Single stranded DNA-binding protein [Shewanella hanedai] |
| gi\|483597 | Single-stranded DNA binding protein [Pseudomonas aeruginosa] |
| gi\|264475 | SSb = 12 kda basic functional DNA binding region of 30 kda single- |
| | stranded nucleic-acid-specific acidic protein {N-terminal} [Pisum |
| | sativum = peas, cv. Arkel, Peptide Chloroplast Partial, 20 aa] |
| gi\|264474 | SSB = 28 kda single-stranded nucleic-acid-specific acidic protein |
| | {N-terminal} [Pisum sativum = peas, cv. Arkel, Peptide Chloroplast Partial, 17 |
| | aa] |
| gi\|264473 | SSB = 30 kda single-stranded nucleic-acid-specific acidic protein |
| | {N-terminal} [Pisum sativum = peas, cv. Arkel, Peptide Chloroplast Partial, 25 |
| | aa] |
| gi\|264472 | SSB = 33 kda single-stranded nucleic-acid-specific acidic protein |
| | {N-terminal} [Pisum sativum = peas, cv. Arkel, Peptide Chloroplast Partial, 25 |
| | aa] |
| gi\|254074 | Single-stranded DNA binding protein; SSB [Saccharomyces |
| | cerevisiae] |
| gi\|1097885 | ssDNA-binding protein |
| gi\|225266 | ssb-like ORF 273 |
| gi\|64899 | mitochondrial DNA specific single-stranded DNA binding protein |
| | (mt-SSB) [Xenopus laevis] |
| gi\|47270 | Single-stranded DNA-binding protein [Serratia marcescens] |
| gi\|45638 | Single-stranded DNA-binding protein [Proteus mirabilis] |
| gi\|144656 | Single-stranded DNA-binding protein [Plasmid ColIb-P9] |
| gi\|1107472 | Single stranded DNA binding protein [Plasmid F] |
| gi\|662792 | Single-stranded DNA binding protein [uncultured eubacterium] |
| gi\|507347 | SSB [Haemophilus influenzae] |
| gi\|188856 | Single stranded DNA binding protein [Homo sapiens] |
| gi\|552025 | Single stranded DNA binding protein [Salmonella typhimurium] |
| gi\|147870 | Single-strand DNA-binding protein (ssb) [Escherichia coli] |
| gi\|409951 | Mitochondrial single-stranded DNA-binding protein [Drosophila |
| | melanogaster] |
| gi\|144126 | Single stranded DNA binding protein [Brucella melitensis biovar |
| | Abortus] |

Isolated. With respect to polypeptides, “isolated” refers to a polypeptide that constitutes a major component in a mixture of components, e.g., 30% or more, 40% or more, 50% or more, 60% or more, 70% or more, 80% or more, 90% or more, or 95% or more by weight. Isolated polypeptides typically are obtained by purification from an organism that contains the polypeptide (e.g., a transgenic organism that expresses the polypeptide), although chemical synthesis is also feasible. Methods of polypeptide purification include, for example, ammonium sulfate precipitation, chromatography and immunoaffinity techniques.

A polypeptide of the invention can be detected by any means known in the art, including sodium dodecyl sulphate (SDS)-polyacrylamide gel electrophoresis followed by Coomassie Blue-staining or Western blot analysis using monoclonal or polyclonal antibodies that have binding affinity for the polypeptide to be detected.

Thermostable. “Thermostable” refers to an enzyme or protein (e.g., DNAP, RT and SSB) that is resistant to inactivation by heat. In general, a thermostable enzyme is more resistant to heat inactivation than a mesophilic enzyme. Thus, the nucleic acid synthesis activity or single stranded binding activity of thermostable enzyme or protein may be reduced by heat treatment to some extent, but not as much as mesophilic enzyme or protein.

A thermostable DNAP retains at least 50% (e.g., at least 60%, at least 70%, at least 80%, at least 90%, and at least 95%) of its nucleic acid synthetic activity after being heated in a nucleic acid synthesis mixture at 90° C. for 30 seconds. In contrast, mesophilic DNAPs lose most of their nucleic acid synthetic activity after such heat treatment. Thermostable DNAPs typically also have a higher optimum nucleic acid synthesis temperature than the mesophilic T5 DNAP.

Thermostable SSBs bind ssDNA at 70° C. at least 70% (e.g., at least 80%, at least 85%, at least 90%, and at least 95%) as well as they do at 37° C. The degree to which an SSB binds ssDNA at such temperatures can be determined by measuring intrinsic SSB fluorescence. Intrinsic SSB fluorescence is related to conserved OB fold amino acids, and is quenched upon binding to ssDNA (see e.g., Alani, E. et al. (1992) J. Mol. Biol. 227:54-71). A routine protocol for determining SSB-ssDNA binding is described in Kelly, T. et al. (1998) Proc. Natl. Acad. Sci. USA 95:14634-14639. Briefly, SSB-ssDNA binding reactions are performed in 2 ml buffer containing 30 mM HEPES (pH 7.8), 100 mM NaCl, 5 mM MgCl₂, 0.5% inositol and 1 mM DTT. A fixed amount of SSB is incubated with varying quantities of poly(dT), and fluorescence is measured using an excitation wavelength of about 295 nm and an emission wavelength of about 348 nm.

Fidelity. Fidelity refers to the accuracy of nucleic acid polymerization; the ability of DNAP or RT to discriminate correct from incorrect substrates (e.g., nucleotides) when synthesizing nucleic acid molecules which are complementary to a template. The higher the fidelity, the less the enzyme misincorporates nucleotides in the growing strand during nucleic acid synthesis. Thus, an increase or enhancement in fidelity results in more faithful nucleic acid synthesis by DNAP or RT, with decreased misincorporation.

Increased/enhanced/higher fidelity means having an increase in fidelity, preferably about 1.2 to about 10,000 fold, about 1.5 to about 10,000 fold, about 2 to about 5,000 fold, or about 2 to about 2000 fold (preferably greater than about 5 fold, more preferably greater than about 10 fold, still more preferably greater than about 50 fold, still more preferably greater than about 100 fold, still more preferably greater than about 500 fold and most preferably greater than about 100 fold) reduction in the number of misincorporated nucleotides during synthesis of a nucleic acid of given length compared to the fidelity of a control DNAP or RT (e.g., in the absence of SSBs) during nucleic acid synthesis.

Reduced misincorporation means less than 90%, less than 85%, less than 75%, less than 70%, less than 60%, or preferably less than 50%, preferably less than 25%, more preferably less than 10%, and most preferably less than 1% of relative misincorporation compared to a control DNAP or RT (e.g., in the absence of SSBs) during nucleic acid synthesis.

Homologs and variants. DNAP, RT and SSB polypeptides suitable for the compositions and methods of the invention can be identified by homologous nucleotide and polypeptide sequence analyses. Known polypeptides in one organism can be used to identify homologous polypeptides in another organism. For example, performing a query on a database of nucleotide or polypeptide sequences can identify homologs of a known polypeptide. Homologous sequence analysis can involve BLAST or PSI-BLAST analysis of databases using known polypeptide amino acid sequences. Those proteins in the database that have greater than 35% sequence identity are candidates for further evaluation for suitability in the compositions and methods of the invention. If desired, manual inspection of such candidates can be carried out in order to narrow the number of candidates that can be further evaluated. Manual inspection is performed by selecting those candidates that appear to have domains conserved among known polypeptides.

A percent identity for any subject nucleic acid or amino acid sequence relative to another “target” nucleic acid or amino acid sequence can be determined as follows. First, a target nucleic acid or amino acid sequence can be compared and aligned to a subject nucleic acid or amino acid sequence, using the BLAST 2 Sequences (Bl2seq) program from the stand-alone version of BLASTZ containing BLASTN and BLASTP (e.g., version 2.0.14). The stand-alone version of BLASTZ can be obtained at <www.fr.com/blast> or at <www.ncbi.nlm.nih.gov>. Instructions explaining how to use BLASTZ, and specifically the Bl2seq program, can be found in the ‘readme’ file accompanying BLASTZ. The programs also are described in detail by Karlin et al. (1990) Proc. Natl. Acad. Sci. 87:2264; Karlin et al. (1993) Proc. Natl. Acad. Sci. 90:5873; and Altschul et al. (1997) Nucl. Acids Res. 25:3389.

Bl2seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per position cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acids (i.e., excluding gaps) from the target sequence that align with sequence from the subject sequence starting with any matched position and ending with any other matched position. A matched position is any position where an identical nucleotide or amino acid is present in both the target and subject sequence. Gaps of one or more positions can be inserted into a target or subject sequence to maximize sequence alignments between structurally conserved domains.

The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the Bl2seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90). In some embodiments, the amino acid sequence of a suitable homolog or variant has 40% sequence identity to the amino acid sequence of a known polypeptide. It will be appreciated that a nucleic acid or amino acid target sequence that aligns with a subject sequence can result in many different lengths with each length having its own percent identity. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

In some embodiments, the amino acid sequence of a suitable homolog or variant has greater than 40% sequence identity (e.g., >80%, >70%, >60%, >50% or >40% to the amino acid sequence of a known polypeptide.

The identification of conserved regions in a subject polypeptide can facilitate homologous polypeptide sequence analysis. Conserved regions can be identified by locating a region within the primary amino acid sequence of a subject polypeptide that is a repeated sequence, forms a secondary structure (e.g., alpha helices and beta sheets), establishes positively or negatively charged domains, or represents a protein motif or domain. See, e.g., the Pfam web site describing consensus sequences for a variety of protein motifs and domains at http://www.sanger.ac.uk/Pfam/ and http://genome.wustl.edu/Pfam/. A description of the information included at the Pfam database is described in Sonnhammer et al. (1998) Nucl. Acids Res. 26:320-322; Sonnhammer et al. (1997) Proteins 28:405-420; and Bateman et al. (1999) Nucl. Acids Res. 27:260-262. From the Pfam database, consensus sequences of protein motifs and domains can be aligned with the template polypeptide sequence to determine conserved region(s). Other methods for identifying conserved regions in a subject polypeptide are described, e.g., in Bouckaert et al. U.S. Ser. No. 60/121,700, filed Feb. 25, 1999.

Typically, polypeptides that exhibit at least about 35% amino acid sequence identity are useful to identify conserved regions. Conserved regions of related proteins sometimes exhibit at least 40% amino acid sequence identity (e.g., at least 50%, at least 60%, at least 70%, at least 80%, or at least 90% amino acid sequence identity). In some embodiments, a conserved region of target and template polypeptides exhibit at least 92, 94, 96, 98, or 99% amino acid sequence identity. Amino acid sequence identity can be deduced from amino acid or nucleotide sequence.

Some variants of known proteins suitable for use in the compositions and methods of the invention have an amino acid sequence with substitutions, insertions or deletions relative to a known polypeptide or homolog. Thus, in some embodiments, the amino acid sequence of a polypeptide corresponds to less than the full-length sequence (e.g. a conserved or functional domain) of a known polypeptide or homolog.

One of skill in the art can make “conservatively modified variants” by making individual substitutions, deletions or additions to a polypeptide that alter, add or delete a single amino acid or a small percentage of amino acids in the encoded sequence where the alteration results in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see e.g., Creighton, Proteins (1984)).

Vector. A vector is a nucleic acid such as a plasmid, cosmid, phage, or phagemid that can replicate autonomously in a host cell. A vector has one or a small number of sites that can be cut by a restriction endonuclease in a determinable fashion, and into which DNA can be inserted. A vector also can include a marker suitable for use in identifying hosts that contain the vector. Markers confer a recognizable phenotype on host cells in which such markers are expressed. Commonly used markers include antibiotic resistance genes such as those that confer tetracycline resistance or ampicillin resistance. Vectors also can contain sequences encoding polypeptides that facilitate the introduction of the vector into a host. Such polypeptides also can facilitate the maintenance of the vector in a host.

“Expression vectors” include nucleic acid sequences that can enhance and/or regulate the expression of inserted DNA, after introduction into a host. Expression vectors contain one or more regulatory elements operably linked to a DNA insert. Such regulatory elements include promoter sequences, enhancer sequences, response elements, protein recognition sites, or inducible elements that modulate expression of a nucleic acid. As used herein, “operably linked” refers to positioning of a regulatory element in a vector relative to a DNA insert in such a way as to permit or facilitate transcription of the insert and/or translation of resultant RNA transcripts. The choice of element(s) included in an expression vector depends upon several factors, including, replication efficiency, selectability, inducibility, desired expression level, and cell or tissue specificity.

Host. The term “host” includes prokaryotes, such as E. Coli, and eukaryotes, such as fungal, insect, plant and animal cells. Animal cells include, for example, COS cells and HeLa cells. Fungal cells include yeast cells, such as Saccharomyces cereviseae cells. A host cell can be transformed or transfected with a vector using techniques known to those of ordinary skill in the art, such as calcium phosphate or lithium acetate precipitation, electroporation, lipofection and particle bombardment. Host cells that contain a vector or portion thereof (a.k.a. “recombinant hosts”) can be used for such purposes as propagating the vector, producing a nucleic acid (e.g., DNA, RNA, antisense RNA) or expressing a polypeptide. In some cases, a recombinant host contains all or part of a vector (e.g., a DNA insert) on the host genome.

Nucleic acid synthesis compositions. The invention provides nucleic acid synthesis compositions that include one or more anti-DNAP antibodies and/or one or more anti-RT antibodies and/or one or more SSBs (or combinations thereof). In particular, the invention provides compositions that contain one or more temperature sensitive anti-DNAP antibodies, one or more temperature sensitive anti-RT antibodies and/or one or more SSBs. Preferably, one or more thermostable SSBs are used in the invention. In some embodiments, nucleic acid synthesis compositions include one or more temperature sensitive anti-DNAP antibodies and one or more thermostable SSBs. In another aspect, the nucleic acid synthesis compositions include temperature sensitive anti-RT antibodies and are one or more SSBS. In some embodiments, nucleic acid synthesis compositions of the invention include two or more SSBs, which preferably are thermostable SSBs.

Nucleic acid synthesis compositions in accord with the invention also can include one or more DNAPs (preferably thermostable DNAPs), one or more nucleotides, one or more primers, and/or one or more templates. In some embodiments, a nucleic acid synthesis reaction can include mRNA and an enzyme having reverse transcriptase activity.

Methods for synthesizing nucleic acids. Compositions of the invention can be used to improve the yield and/or homogeneity of primer extension products made by DNAP during nucleic acid synthesis (e.g., during first strand synthesis, cDNA synthesis, amplification and combined cDNA synthesis/amplification reactions).

Compositions of the invention may be used, e.g., in “hot-start” nucleic acid synthesis, where a reaction is set up at a temperature such that anti-DNAP antibodies and/or anti-RT antibodies can exhibit nucleic acid synthesis and where nucleic acid synthesis subsequently is initiated by increasing the temperature to reduce inhibition by the anti-DNAP antibodies and/or anti-RT antibodies. Thus, the invention provides a method for synthesizing a nucleic acid involving: (a) mixing one or more templates with one or more anti-DNAP antibodies and/or one or more anti-RT antibodies and/or one or more SSBs (or combinations thereof) to form a mixture; (b) incubating the mixture under conditions sufficient to inhibit or prevent nucleic acid synthesis; and (c) incubating the mixture under conditions sufficient to make one or more nucleic acid molecules complementary to all or a portion of said templates (i.e., a primer extension product). Reaction conditions sufficient to allow nucleic acid synthesis (e.g., pH, temperature, ionic strength, and incubation time) can be optimized according to routine methods known to those skilled in the art and may involve the use of one or more primers, one or more nucleotides, one or more buffers or buffering salts, one or more RTs and/or one or more DNAPs (or combinations thereof).

In one aspect, a nucleic acid method of the invention may comprise mixing one or more templates with one or more anti-DNAP antibodies and/or one or more anti-RT antibodies and/or one or more SSBs to form a mixture, and incubating the mixture under conditions sufficient to make one or more nucleic acid molecules complementary to all or a portion of said templates. Such conditions may involve the use of one or more primers, one or more nucleotides, one or more buffers or buffering salts, one or more RTs and/or one or more DNAPs (or combinations thereof). Conditions to facilitate nucleic acid synthesis such as pH, ionic strength, temperature and incubation time can be determined as a matter of routine by those skilled in the art.

In one embodiment, a nucleic acid molecule is synthesized by mixing one or more templates, one or more thermostable DNAPs, one or more temperature sensitive anti-DNAP antibodies, and one or more thermostable SSBs to form a mixture. In another embodiment, nucleic acid synthesis is accomplished by mixing one or more templates, one or more RTs, one or more temperature sensitive anti-RT antibodies and one or more SSBs to form a mixture. Synthesis of a nucleic acid molecule complementary to all or a portion of the template is accomplished after raising the temperature of the reaction and thereby reducing inhibition of DNAP by anti-DNAP antibodies and/or by reducing inhibition of RT by anti-RT antibodies. Nucleic acid synthesis is accomplished in the presence of nucleotides (e.g., deoxyribonucleoside triphosphates (dNTPs) and/or dideoxyribonucleoside triphosphate (ddNTPs) or derivatives thereof).

In another aspect, the invention provides a method for synthesizing a nucleic acid involving: (a) mixing one or more templates with two or more (three or more, four or more, five or more, six or more, etc.) SSBs to form a mixture; and (b) incubating the mixture under conditions sufficient to make a nucleic acid complementary to all or a portion of the templates (i.e., a primer extension product). Reaction conditions sufficient to allow nucleic acid synthesis (e.g., pH, temperature, ionic strength, and incubation time) can be optimized according to routine methods known to those skilled in the art and may involve the use of one or more primers, one or more nucleotides, one or more buffers or buffering salts, one or more RTs and/or one or more DNAPs (or combinations thereof).

The invention also provides a method for amplifying a nucleic acid involving: (a) mixing one or more templates with one or more anti-DNAP antibodies (and optionally one or more anti-RT antibodies), and one or more thermostable SSBs to form a mixture; (b) incubating the mixture under conditions sufficient to inhibit or prevent nucleic acid amplification; and (c) incubating the mixture under conditions sufficient to allow the one or more DNAPs to amplify a nucleic acid molecule complementary to all or a portion of the template. Reaction conditions sufficient to allow nucleic acid synthesis (e.g., pH, temperature, ionic strength, and incubation time) can be optimized according to routine methods known to those skilled in the art and may involve the use of one or more primers, one or more nucleotides, one or more buffers or buffering salts, one or more RTs and/or one or more DNAPs (or combinations thereof).

In one embodiment, a nucleic acid is amplified by mixing one or more templates, one or more thermostable DNAPs (and optionally one or more reverse transcriptases), one or more temperature-sensitive anti-DNAP antibodies (and optionally one or more anti-RT antibodies), and one or more thermostable SSBs to form a mixture. Amplifying a nucleic acid molecule complementary to all or a portion of the templates is accomplished after raising the temperature of the reaction and thereby reducing inhibition of DNAP by anti-DNAP antibodies. Nucleic acid synthesis is accomplished in the presence of nucleotides (e.g., deoxyribonucleoside triphosphates (dNTPs), dideoxyribonucleoside triphosphate (ddNTPs) or derivatives thereof).

In another aspect, the invention provides a method for amplifying a nucleic acid involving: (a) mixing one or more templates with two or more SSBs to form a mixture; and (b) incubating the mixture under conditions sufficient to amplify a nucleic acid complementary to all or a portion of the templates. Such conditions may involve the use of one or more primers, one or more nucleotides, one or more buffers or buffering salts, one or more RTs and/or one or more DNAPs (or combinations thereof). Conditions to facilitate nucleic acid synthesis such as pH, ionic strength, temperature and incubation time can be determined as a matter of routine by those skilled in the art.

Nucleic acid amplification methods may involve the use of one or more enzymes having reverse transcriptase activity, in methods known in the art as one-step (e.g., one-step RT-PCR) or two-step (e.g., two-step RT-PCR) reverse transcriptase-amplification reactions. To amplify long nucleic acid molecules (e.g., greater than about 3-5 Kb in length), a combination of DNA polymerases may be used, as disclosed in WO 98/06736 and WO 95/16028.

Following nucleic acid synthesis, nucleic acids can be isolated for further use or characterization. Synthesized nucleic acids can be separated from other nucleic acids and other constituents present in a nucleic acid synthesis reaction by any means known in the art, including gel electrophoresis, capillary electrophoresis, chromatography (e.g., size, affinity and immunochromatography), density gradient centrifugation, and immunoadsorption. Separating nucleic acids by gel electrophoresis provides a rapid and reproducible means of separating nucleic acids, and permits direct, simultaneous comparison of nucleic acids present in the same or different samples. Nucleic acids made by the provided methods can be isolated using routine methods. For example, nucleic acids can be removed from an electrophoresis gel by electroelution or physical excision. Isolated nucleic acids can be inserted into vectors, including expression vectors, suitable for transfecting or transforming prokaryotic or eukaryotic cells.

Some nucleic acid synthesis techniques involve sequencing nucleic acids, e.g., by routine methods known in the art (see e.g., U.S. Pat. Nos. 4,962,022 and 5,498,523). The invention is particularly well-suited for cycle sequencing reactions. Cycle sequencing often involves the use of fluorescent dyes. In some cycle sequencing protocols, sequencing primers are labeled with fluorescent dye (e.g., using Amersham Bioscience MegaBACE DYEnamic ET Primers, ABI Prism® BigDye™ primer cycle sequencing kit, and Beckman Coulter WellRED fluorescence dye). Sequencing reactions using fluorescent primers offers advantages in accuracy and readable sequence length. However, separate reactions must be prepared for each nucleotide base for which sequence position is to be determined. In other cycle sequencing protocols, fluorescent dye is linked to ddNTP as a dye terminator (e.g., using Amersham Bioscience MegaBACE DYEnamic ET Terminator cycle sequencing kit, ABI Prism® BigDye™ Terminator cycle sequencing kit, ABI Prism® dRhodamine Terminator cycle sequencing kit, LI-COR IRDye™ Terminator Mix, and CEQ Dye Terminator Cycle sequencing kit with Beckman Coulter WellRED dyes). Since dye terminators can be labeled with unique fluorescence dye for each base, sequencing can be done in a single reaction.

The invention thus provides a method for sequencing a nucleic acid involving: (a) mixing one or more templates to be sequenced with one or more anti-DNAP antibodies, and one or more SSBs (and optionally one or more terminating agents such as ddNTPs) to form a mixture; (b) incubating the mixture under conditions sufficient to inhibit or prevent nucleic acid sequencing or synthesis; (c) incubating the mixture under conditions sufficient to synthesize a population of molecules complementary to all or a portion of the templates to be sequenced; and (d) separating the population to determine the nucleotide sequence of all or a portion of the template to be sequenced. Reaction conditions sufficient to allow nucleic acid synthesis (e.g., pH, temperature, ionic strength, and incubation time) can be optimized according to routine methods known to those skilled in the art and may involve the use of one or more primers, one or more nucleotides, one or more buffers or buffering salts, and/or one or more DNAPs (or combinations thereof).

In one aspect, a sequencing method of the invention may comprise mixing one or more templates to be sequenced with one or more anti-DNAP antibodies and/or one or more SSBs to form a mixture and incubating the mixture under conditions sufficient to make a population of nucleic acid molecules complementary to all or a portion of said templates, and separating the population of nucleic acid molecules to determine the nucleotide sequence of all or a portion of the templates to be sequenced. Such conditions may involve the use of one or more primers, one or more nucleotides, one or more buffers or buffering salts, one or more nucleic acid synthesis terminating agents (e.g., ddNTP), and/or one or more DNAPs (or combinations thereof). Conditions to facilitate nucleic acid synthesis such as pH, ionic strength, temperature and incubation time can be determined as a matter of routine by those skilled in the art.

In one embodiment, a nucleic acid is sequenced by mixing one or more templates to be sequenced with one or more thermostable DNAPs, one or more temperature sensitive anti-DNAP antibodies, and one or more thermostable SSBs to form a mixture. Synthesis of nucleic acid molecules complementary to all or a portion of the templates to be sequenced is accomplished after raising the temperature of the reaction and thereby reducing inhibition of DNAP by anti-DNAP antibodies.

In another aspect, the invention provides a method for sequencing a nucleic acid involving: (a) mixing one or more templates to be sequenced with two or more SSBs (and optionally one or more nucleic acid synthesis terminating agents such as ddNTPs) to form a mixture; (b) incubating the mixture under conditions sufficient to synthesize a population of molecules complementary to all or a portion of the template to be sequenced; and (c) separating the population to determine the nucleotide sequence of all or a portion of the template to be sequenced.

Kits. The invention also provides kits for use in, for example, the synthesis, amplification or sequencing of nucleic acids. Kits can include one or more of the following constituents: one or more DNAPs, one or more RTs, one or more nucleotides, one or more primers, one or more templates, one or more anti-DNAP antibodies, one or more anti-RT antibodies, and one or more SSBs. In some embodiments, kits of the invention include one or more anti-DNAP antibodies and/or one or more anti-RT antibodies and/or one or more SSBs (or combinations thereof). In some embodiments, kits include two or more SSBs. Kits of the invention also can include one or more host cells (which may be competent to uptake nucleic acid molecules such as chemically competent cells or electrocompetent cells). Kits of the invention also can include one or more ligases (preferably DNA ligases such as T4 DNA ligase, one or more topoisomerases (such as type 1A and 1B) and/or one or more vectors. Kit constituents typically are provided, individually or collectively, in containers (e.g., vials, tubes, ampules, and bottles). Kits typically include packaging material, including instructions describing how the kit can be used for example to synthesize, amplify or sequence nucleic acids.

It will be readily apparent to one of ordinary skill in the relevant arts that other suitable modifications and adaptations to the methods and applications described herein may be made without departing from the scope of the invention or any embodiment thereof. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the inventions described in the claims.

### Example 1

### Accuprime™ TAQ DNA Polymerase System

Description: The AccuPrime™ Taq DNA Polymerase System provides qualified reagents for the amplification of nucleic acid templates by polymerase chain reaction (PCR). The AccuPrime™ Taq DNA polymerase contains anti-Taq DNA polymerase antibodies. 10× AccuPrime™ buffers contain thermostable AccuPrime™ protein (i.e., Methanococcus jannachii SSB), Mg⁺⁺, and deoxyribonucleotide triphosphates at concentrations sufficient to allow amplification during PCR. Two individual buffer systems (10× AccuPrime™ PCR Buffer I and II) are provided for amplification of specific types of templates. Reagents sufficient for 200 or 1,000 amplification reactions of 25 μl each are provided.

Anti-Taq DNA polymerase antibodies inhibit polymerase activity providing an automatic “hot start” (Chou, Q. et al. (1992) Nucl. Acids Res. 20:1717; and Sharkey, D. et. al. (1994) BioTechnology 12:506) and permits ambient temperature set-up. The thermostable AccuPrime™ protein enhances specific primer-template hybridization during every cycle of PCR. Antibody/AccuPrime™ protein-mediated amplification dramatically improves PCR specificity. It also improves the fidelity of Taq by 2-fold, and provides robust PCR for multiplex PCR and sub-optimal primer sets.

Formulation:

| | | Amt | |
|---|---|---|---|
| Component | Amt (200 rxn kit) | (1,000 rxn kit) | |
| AccuPrime ™ Taq DNA Polymerase | 100 μl | 500 | μl |
| 10X AccuPrime ™ PCR Buffer I* | 500 μl | 2 × 1.25 | ml |
| 10X AccuPrime ™ PCR Buffer II* | 500 μl | 2 × 1.25 | ml |
| 50 mM Magnesium Chloride | 500 μl | 500 | μl |

*10X AccuPrime ™ PCR Buffer I is designed for small genomic DNA amplicon (≦200 bp), plasmid, or cDNA applications. Use 10X AccuPrime ™ PCR Buffer II for genomic DNA (200 bp-4 kb) applications.

10× AccuPrime PCR Buffer I and II: 200 mM Tris-HCl (pH 8.4), 500 mM KCl, 15 mM MgCl₂, 2 mM dGTP, 2 mM dATP, 2 mM dTTP, 2 mM dCTP, thermostable AccuPrime™ protein (10 ug/ml for Buffer I, 80 ug/ml for Buffer II), 10% glycerol.

Storage Buffer: 20 mM Tris-HCl (pH8.0), 0.1 mM EDTA, 1 mM DTT, stabilizers, 50% (v/v) glycerol.

Quality Control: AccuPrime™ Taq DNA Polymerase is evaluated in a PCR functional assay. AccuPrime™ Taq DNA Polymerase and 10× AccuPrime™ PCR Buffers are functionally tested for amplification. AccuPrime™ Taq DNA Polymerase and AccuPrime™ protein are tested for the absence of double- and single-stranded endonuclease activity as well as the absence of 5′- and 3′-exonuclease activity.

PCR Precautions: Since PCR is a powerful technique capable of amplifying trace amounts of DNA, all appropriate precautions should be taken to avoid cross-contamination. Ideally, amplification reactions should be assembled in a DNA-free environment. Use of aerosol-resistant barrier tips is recommended. Take care to avoid contamination with the primers or template DNA used in individual reactions. PCR products should be analyzed in an area separate from the reaction assembly area.

General Protocol: The following general procedure is suggested as a guideline and as a starting point when using AccuPrime™ Taq DNA Polymerase in any PCR amplification. Optimal reaction conditions (incubation times and temperatures, amount of AccuPrime™ Taq DNA Polymerase, primers, MgCl₂, and template DNA) vary and need to be optimized. Reaction size may be altered to suit user preferences. For general PCR reaction assembly refer to volumes and quantities in column 3 of the following tables. For miniaturization, refer to quantities in columns 1 & 2 of the following tables for recommended miniaturized PCR reaction assembly.

1. Add the following components to a sterile thin wall 0.25-ml or 0.5-ml PCR tube at either ambient temperature, or on ice:

For Small Genomic DNA (≦200 bp), Plasmid or cDNA:

| Component | 10-μl Reaction | | 25-μl Reaction | | 50-μl Reaction | |
|---|---|---|---|---|---|---|
| 10X AccuPrime ™ | 1 | μl | 2.5 | μl | 5 | μl |
| PCR Buffer I | | | | | | |
| Primer Mix (10 μM | 0.2 | μl | 0.5 | μl | 1 | μl |
| each) | | | | | | |
| Template DNA | 10 pg-200 ng | | 10 pg-200 ng | | 10 pg-200 ng | |
| AccuPrime ™ Taq | 0.25 | μl | 0.5 | μl | 1 | μl |
| DNAP | | | | | | |
| Autoclaved distilled | To 10 | μl | To 25 | μl | To 50 | μl |
| water | | | | | | |

For Genomic DNA (200 bp-4 kb):

| Component | 10-μl Reaction | | 25-μl Reaction | | 50-μl Reaction | |
|---|---|---|---|---|---|---|
| 10X AccuPrime ™ | 1 | μl | 2.5 | μl | 5 | μl |
| PCR Buffer II | | | | | | |
| Primer Mix (10 μM | 0.2 | μl | 0.5 | μl | 1 | μl |
| each) | | | | | | |
| Template DNA | 1-200 | ng | 1-200 | ng | 1-200 | ng |
| AccuPrime ™ Taq | 0.25 | μl | 0.5 | μl | 1 | μl |
| DNAP | | | | | | |
| Autoclaved distilled | To 10 | μl | To 25 | μl | To 50 | μl |
| water | | | | | | |

If desired, a master mix can be prepared for multiple reactions, to minimize reagent loss and to enable accurate pipetting.

2. Mix contents of the tubes and overlay with 50 μl of mineral or silicone oil, if necessary.

3. Cap the tubes and centrifuge briefly to collect the contents.

4. Incubate tubes in a thermal cycler at 94° C. for 2 min to completely denature the template and activate the enzyme.

5. Perform 25-35 cycles of PCR amplification as follows:
Denature: 94° C. for 15-30 s
Anneal: 55° C.-60° C. for 15-30 s
Extend: 68° C. for 1 min per kb

6. Maintain the reaction at 4° C. after cycling. The samples can be stored at −20° C. until use.

7. Analyze the amplification products by agarose gel electrophoresis and visualize by ethidium bromide staining. Use appropriate molecular weight standards.

Specialized Protocols: The following specialized procedure is suggested as a guideline and as a starting point when using AccuPrime™ Taq DNA Polymerase in Multiplex PCR amplification. Optimal reaction conditions (incubation times and temperatures, amount of AccuPrime™ Taq DNA Polymerase, primers, MgCl₂, and template DNA) vary and need to be optimized. Reaction size may be altered to suit user preferences.

Add the following components to a sterile thin wall 0.25-ml or 0.5-ml PCR tube at either ambient temperature, or on ice:

| For small genomic Amplicon | | | |
|---|---|---|---|
| (≦200 bp), cDNA or Plasmid | | For genomic DNA (200 bp-4 kb) | |
| Components | Amount | Components | Amount |
| 10X | 5 μl | 10X AccuPrime ™ | 5 μl |
| AccuPrime ™ | | PCR Buffer II | |
| PCR Buffer I | | | |
| Primer mix | 1 μl each | Primer mix | 1 μl each (0.2 μM |
| (10 μM each) | (0.2 μM | (10 μM each) | each) |
| | each) | | |
| Template DNA | 100-200 ng | Template DNA | 100-200 ng |
| AccuPrime ™ | 1-2.5 μl | AccuPrime ™ Taq | 1-2.5 μl |
| Taq DNA | | DNA Polymerase | |
| Polymerase | | | |
| Autoclaved, | to 50 μl | Autoclaved, | to 50 μl |
| distilled water | | distilled water | |

For primer mixes up to 5 sets, 1 μl of enzyme is sufficient. If desired, a master mix can be prepared for multiple reactions, to minimize reagent loss and to enable accurate pipetting. Continue with steps 2-7 of the General Protocol.

### Example 2

### Accuprime™ Supermix II

AccuPrime™ SuperMix II is designed for amplification of genomic DNA (200 bp-4 kb) templates.

Description: AccuPrime™ SuperMix II provides reagents for the amplification of nucleic acid templates by polymerase chain reaction (PCR). The mixture contains anti-Taq DNA polymerase antibodies, thermostable AccuPrime™ protein (i.e., Methanococcus jannachii SSB), Mg⁺⁺, deoxyribonucleotide triphosphates, and recombinant Taq DNA polymerase at concentrations sufficient to allow amplification during PCR. AccuPrime™ SuperMix II is supplied at 2× concentration to allow 50% of the final reaction volume to be used for the addition of primer and template solutions. Reagents sufficient for 200 or 1,000 amplification reactions of 25 μl each are provided.

Anti-Taq DNA polymerase antibodies inhibit polymerase activity providing an automatic “hot start” (Chou, Q. et al. (1992) Nucl. Acids Res. 20:1717; and Sharkey, D. et. al. (1994) BioTechnology 12.506) and permits ambient temperature set-up. The thermostable AccuPrime™ protein enhances specific primer-template hybridization during every cycle of PCR. Antibody/AccuPrime™ protein-mediated amplification dramatically improves PCR specificity. It also improves the fidelity of Taq by 2-fold, and provides the most robust PCR for multiplex PCR and sub-optimal primer sets.

AccuPrime™ SuperMix II may be stored at either −20° C. or 4° C. Storage at 4° C. avoids the necessity of thawing the mix before assembling the PCR. No detectable reduction of PCR performance or enzyme activity is observed after storage of AccuPrime™ SuperMix II for twelve months at 4° C. Repeated freeze-thaw cycles can reduce performance or activity.

Configuration:

| No. reactions | Component | No. tubes | Amt./tube |
|---|---|---|---|
| 200 reactions | AccuPrime ™ SuperMix II | 2 | 1.25 ml |
| 1,000 reactions | AccuPrime ™ SuperMix II | 1 | 12.5 ml |

AccuPrime™ SuperMix II: 40 mM Tris-HCl (pH 8.4), 100 mM KCl, 3 mM MgCl₂, 400 μM dGTP, 400 μM dATP, 400 μM dTTP, 400 μM dCTP, AccuPrime™ Taq DNA Polymerase, thermostable AccuPrime™ protein, stabilizers.

Quality Control: AccuPrime™ SuperMix II is evaluated in a PCR functional assay. Components of AccuPrime™ SuperMix II are tested for the absence of DNase, RNase, and exonuclease activities. AccuPrime™ Taq DNA polymerase and AccuPrime™ protein are tested for the absence of exonuclease, and double- and single-stranded endonuclease activities. The enzyme is >90% homogeneous as determined by SDS-polyacrylamide gel electrophoresis.

PCR Precautions: Since PCR is a powerful technique capable of amplifying trace amounts of DNA, all appropriate precautions should be taken to avoid cross-contamination. Ideally, amplification reactions should be assembled in a DNA-free environment.

General Protocol: The following general procedure is suggested as a guideline and as a starting point when using AccuPrime™ SuperMix II in any PCR amplification. Optimal reaction conditions (incubation times and temperatures, primers, and template DNA) vary and need to be optimized. Reaction size may be altered to suit user preferences.

Recommended starting volumes for AccuPrime™ SuperMix II:

| Component | 10-μl Rxn | 25-μl Rxn | 50-μl Rxn |
|---|---|---|---|
| AccuPrime ™ | 5 μl | 12.5 μl | 25 μl |
| SuperMix II | | | |
| Primer mix | 0.2 μl (0.2 μM | 0.5 μl (0.2 μM | 1 μl (0.2 μM |
| (10 μM each) | each) | each) | each) |
| Template | 10 pg-200 ng | 10 pg-200 ng | 10 pg-200 ng |
| DNA | | | |
| DNase-free | Up to 10 μl | Up to 25 μl | Up to 50 μl |
| H₂O | | | |

PCR Assembly from Reaction Components:

1. Using the chart above as a guide, add the following components in any order to each reaction:
a. AccuPrime™ SuperMix II
b. Primer solution (200 nM final concentration of each is recommended)
c. Template DNA
d. DNase-free H₂O to final total volume.

2. Mix contents of tubes and cover with mineral or silicone oil if necessary.

3. Cap tubes and centrifuge briefly to collect the contents to the bottom of the tubes.

4. Incubate tubes in a thermal cycler at 94° C. for 2 min to completely denature the template and activate the enzyme.

5. Perform 25-35 cycles of PCR amplification as follows:
Denature: 94° C. for 15-30 s
Anneal: 55° C.-60° C. for 15-30 s
Extend: 68° C. for 1 min per kb

6. Maintain the reaction at 4° C. after cycling. The samples can be stored at −20° C. until use.

7. Analyze the amplification products by agarose gel electrophoresis and visualize by ethidium bromide staining. Use appropriate molecular weight standards.

### Example 3

### Accuprime™ Supermix I

AccuPrime™ SuperMix I is designed for amplification of genomic DNA amplicons (≦200 bp), plasmid DNA, or cDNA templates.

Description: AccuPrime™ SuperMix I provides qualified reagents for the amplification of nucleic acid templates by polymerase chain reaction (PCR). The mixture contains anti-Taq DNA polymerase antibodies, thermostable AccuPrime™ protein (i.e., Methanococcus jannachii SSB), Mg⁺⁺, deoxyribonucleotide triphosphates, and recombinant Taq DNA polymerase at concentrations sufficient to allow amplification during PCR. AccuPrime™ SuperMix I is supplied at 2× concentration to allow 50% of the final reaction volume to be used for the addition of primer and template solutions. Reagents sufficient for 200 or 1,000 amplification reactions of 25 μl each are provided.

Anti-Taq DNA polymerase antibodies inhibit polymerase activity providing an automatic “hot start” (Chou, Q. et al. (1992) Nucl. Acids Res. 20:1717; and Sharkey, D. et. al. (1994) BioTechnology 12:506) and permits ambient temperature set-up. The thermostable AccuPrime™ protein enhances specific primer-template hybridization during every cycle of PCR. Antibody/AccuPrime™ protein-mediated amplification dramatically improves PCR specificity. It also improves the fidelity of Taq by 2-fold, and provides the most robust PCR for multiplex PCR and sub-optimal primer sets.

AccuPrime™ SuperMix I may be stored at either −20° C. or 4° C. Storage at 4° C. avoids the necessity of thawing the mix before assembling the PCR. No detectable reduction of PCR performance or enzyme activity is observed after storage of AccuPrime™ SuperMix I for twelve months at 4° C. Repeated freeze-thaw cycles can reduce performance or activity.

Configuration:

| No. reactions | Component | No. tubes | Amt./tube |
|---|---|---|---|
| 200 reactions | AccuPrime ™ SuperMix I | 2 | 1.25 ml |
| 1,000 reactions | AccuPrime ™ SuperMix I | 1 | 12.5 ml |

AccuPrime™ SuperMix I: 40 mM Tris-HCl (pH 8.4), 100 mM KCl, 3 mM MgCl₂, 400 μM dGTP, 400 μM dATP, 400 μM dTTP, 400 μM dCTP, AccuPrime™ Taq DNA Polymerase, thermostable AccuPrime™ protein, stabilizers.

Quality Control: AccuPrime™ SuperMix I is evaluated in a PCR functional assay. Components of AccuPrime™ SuperMix I are tested for the absence of DNase, RNase, and exonuclease activities. AccuPrime™ Taq DNA polymerase and AccuPrime™ protein are tested for the absence of exonuclease, and double- and single-stranded endonuclease activities. The enzyme is >90% homogeneous as determined by SDS-polyacrylamide gel electrophoresis.

PCR Precautions: Since PCR is a powerful technique capable of amplifying trace amounts of DNA, all appropriate precautions should be taken to avoid cross-contamination. Ideally, amplification reactions should be assembled in a DNA-free environment.

General Protocol: The following general procedure is suggested as a guideline and as a starting point when using AccuPrime™ SuperMix I in any PCR amplification. Optimal reaction conditions (incubation times and temperatures, primers, and template DNA) vary and need to be optimized. Reaction size may be altered to suit user preferences.

Recommended starting volumes for AccuPrime™ SuperMix I:

| Component | 10-μl Rxn | 25-μl Rxn | 50-μl Rxn |
|---|---|---|---|
| AccuPrime ™ | 5 μl | 12.5 μl | 25 μl |
| SuperMix I | | | |
| Primer mix | 0.2 μl (0.2 μM | 0.5 μl (0.2 μM | 1 μl (0.2 μM |
| (10 μM each) | each) | each) | each) |
| Template DNA | 1-200 ng | 1-200 ng | 1-200 ng |
| DNase-free H₂O | Up to 10 μl | Up to 25 μl | Up to 50 μl |

PCR Assembly from Reaction Components:

1. Using the chart above as a guide, add the following components in any order to each reaction:
a. AccuPrime™ SuperMix I
b. Primer solution (200 nM final concentration of each is recommended)
c. Template DNA
d. DNase-free H₂O to final total volume.

2. Mix contents of tubes and cover with mineral or silicone oil if necessary.

3. Cap tubes and centrifuge briefly to collect the contents to the bottom of the tubes.

4. Incubate tubes in a thermal cycler at 94° C. for 2 min to completely denature the template and activate the enzyme.

5. Perform 25-35 cycles of PCR amplification as follows:
Denature: 94° C. for 15-30 s
Anneal: 55° C.-60° C. for 15-30 s
Extend: 68° C. for 1 min per kb

6. Maintain the reaction at 4° C. after cycling. The samples can be stored at −20° C. until use.

7. Analyze the amplification products by agarose gel electrophoresis and visualize by ethidium bromide staining. Use appropriate molecular weight standards.

### Example 4

### Development and Characterization of the Accuprime™TAQ DNA Polymerase System

Introduction

A highly thermostable single stranded binding protein derived from Archaea (see U.S. application No. 60/149,680) which we call here AccuPrime protein has been successfully integrated with antibodies specific to DNA polymerases (e.g. Taq DNA polymerase) to generate a next generation amplification enzyme—AccuPrime Taq™ DNA polymerase. We have optimized the AccuPrime Taq™ DNA polymerase (which includes single stranded binding protein from Archaea, Taq DNA polymerase and two different Taq antibodies) for PCR applications that requires high specificity, high sensitivity and robustness. Such applications include multiplex PCR, genotyping, colony PCR, high-throughput PCR and PCR miniaturization.

We find that the AccuPrime protein enhances the activity of Taq DNA polymerase and in PCR improves the specificity drastically. Unlike other hot-start DNA polymerases, it improves PCR performance by promoting specific primer-template hybridization before as well as during every cycle of PCR. All commercially available hot-start Taq DNA polymerase, either by chemically modification or anti-Taq antibody addition, designed to block DNA polymerase activity before PCR cycle but not during PCR cycles. In a PCR study using more than 300 primer sets, AccuPrime Taq™ DNA polymerase showed improvement in yield, sensitivity and/or specificity over other hot-start PCR enzymes in 75% of the cases. While its sensitivity and specificity makes the new amplification enzyme ideal to variety of PCR/RT-PCR applications, its robustness reduces the need for optimization to the minimum for any particular PCR application. In high throughput or multiplex format, such as, genotyping, colony PCR and PCR miniaturization, the new PCR enzyme out-performs all premier gold standard enzymes in the current PCR market. It is also demonstrated that AccuPrime protein improves the fidelity of Taq DNA polymerase by at least 2 fold.

It was believed that single stranded DNA binding protein (SSB) would help in PCR in terms of the specificity, yield and sensitivity, on the basis of the function of the protein in DNA replication system. In DNA replication, helicases unwind double stranded (ds) DNA into two complementary single strands (ss), necessary for the functions of primases and DNA polymerases. SSB protects ssDNA template simply by coating the molecules and, while doing so, prevents the ssDNA from base pairing with the complementary strand. There also exists a set of evidence that SSB may directly interact with DNA polymerases in a species-specific manner (Kim et al., 1992; Kim and Richardson, 1994; Glover and McHenry, 1998; Lee et al., 1998).

The most obvious reason for SSB to enhance PCR reaction would be its ability to remove secondary structures (hairpins and such) from the template, and to maintain the DNA template single-stranded. In fact, it has been reported that SSB from E. coli and other mesophilic organisms improved PCR efficiency (Chou, 1992; Rapley, 1994; Dabrowski and Kur, 1999). However, due to the thermo-labile nature of the mesophilic SSB, the enhancement by the proteins were too limited to be practical in PCR application where the cycling incubation temperatures exceed the upper limit of their thermostability.

The existence of a thermostable SSB from an archaeon was first reported by Dr. Stephen C. Kowalczykowski from UC, Davis (Chedin et al., 1998) and its gene was subsequently cloned by Thomas J. Kelly's group in the Johns Hopkins University (Kelly et al., 1998). We used the SSB from UC, Davis (the protein will be referred to as “AccuPrime protein” hereon) and studied its effect on DNA polymerase activity and fidelity. This manuscript reports our endeavor in creating a next generation PCR amplification technology. The new technology offers PCR specificity improvement in every cycles of PCR unlike the hot start technology where it functions up to the start of PCR cycle.

Materials and Methods

Small Scale Purification of AccuPrime protein

The plasmid containing the AccuPrime protein gene was provided by Dr. Steve Kowalczykowski at UC Davis. The plasmid was transformed into BL21(DE3) cells freshly for each protein purification. A single colony from the transformation plate was used to inoculate a starter culture of 500 ml. The media used was Terrific Broth (Life Technologies), supplemented with 50 μl Kanamycin. The starter culture was incubated at 37° C. overnight, and used in its entirely to inoculate 10 liter TB+Kan media. The culture was incubated at 37° C. to the 1 OD₆₀₀ (4 to 6 hours), induced with 1 mM IPTG, and incubation continued for another 2.5 hrs. Cells were pelleted by centrifugation at 3,000g for 20 min. at 4° C.

Cells were resuspended in Lysis buffer (2 ml per g of cell pellet; 0.5M NaCl, 50 mM potassium phosphate, pH8.0, 0.25 mM PMSF, 10 mM imidazole), containing the protease inhibitor cocktail (Sigma, P 8849; 1 ml of the cocktail per 20 g of cell pellet). Cells were lysed by sonication (10 cycles of 10 sec pulses with a quarter inch probe at 80% power, or continued until >80% lysis). Cell debris was removed by centrifugation at 23,000 g (14,000 rpm in a SS34 rotor) for 1 hr at 4° C. The supernatant was loaded onto a Ni-NTA agarose column.

A Ni-NTA agarose (Qiagen) column (20 ml resin volume) was equilibrated with equilibration buffer with the protease inhibitor cocktail (0.5 M NaCl, 50 mM potassium phosphate, pH8.0, 0.25 mM PMSF, 20 mM imidazole; 1 ml of the inhibitor cocktail per 1 liter of buffer). The column was washed with 10 column volumes (200 ml) of low imidazole buffer (1 M NaCl, 50 mM potassium phosphate, pH8.0, 0.25 mM PMSF, 20 mM imidazole; 1 ml of the inhibitor cocktail per 1 liter of buffer). The protein was eluted with a high imidazole buffer (1 M NaCl, 50 mM potassium phosphate, pH8.0, 0.25 mM PMSF, 250 mM imidazole; 1 ml of the inhibitor cocktail per 1 liter of buffer) in 4 ml fractions. Fractions containing AccuPrime protein (monitored by SDS-PAGE) were pooled and dialyzed into low salt ssDNA agarose column buffer (1 M NaCl, 25 mM Tris-HCl, pH7.5, 1 mM EDTA, 1 mM DTT, 0.25 mM PMSF, 10% glycerol) at 4° C. overnight.

The dialyzed fraction pool was loaded to a ssDNA agarose column (20 ml resin volume) pre-equilibrated with low salt ssDNA agarose column buffer (1 M NaCl, 25 mM Tris-HCl, pH7.5, 1 mM EDTA, 1 mM DTT, 0.25 mM PMSF, 10% glycerol). The column was washed with 10 column volumes (200 ml) of the low salt buffer. The protein was eluted with the high salt ssDNA column buffer (2.5 M NaCl, 40% ethylene glycol, 25 mM Tris-HCl, pH7.5, 1 mM EDTA, 1 mM DTT, 0.25 mM PMSF, 10% glycerol) in 5 ml fractions. The fractions containing the protein were pooled and dialyzed into low salt monoQ column buffer (50 mM NaCl, 25 mM Tris-HCl, pH8.0, 1 mM EDTA, 1 mM DTT, 5% glycerol).

The pool of ssDNA column fractions, dialyzed into the low salt monoQ column buffer was loaded into a MonoQ (5/5) column (Pharmacia, 1 ml resin volume) pre-equilibrated with low salt monoQ column buffer (50 mM NaCl, 25 mM Tris-HCl, pH8.0, 1 mM EDTA, 1 mM DTT, 5% glycerol). The column was operated in a FPLC with the flow rate set at 1 ml/min. The column was washed with 10 column volumes (10 ml) of the low salt buffer and eluted with 20 column volumes of a linear gradient of salt (50 to 1000 mM NaCl), collecting 1 ml fractions. The fractions containing the protein were pooled and dialyzed into the storage buffer (100 mM NaCl, 25 mM Tris-HCl, pH7.5, 1 mM EDTA, 1 mM DTT, 10% glycerol).

Large Scale Purification of AccuPrime Protein

The AccuPrime protein gene was modified (to eliminate an internal ribosome binding site), re-cloned into a pET vector under T7 promoter and transformed into BL21(DE3) pLysP cells. A large-scale culture (120 liter) was grown from a starter culture in Buffered Rich media using a Fermanta. The culture was incubated at 37° C. to the 2.5 to 3 OD₆₀₀, induced with 1 mM IPTG, and incubation continued for another 4 hrs. Cells were pelleted by centrifugation at 3,000 g for 20 min at 4° C. (1.7 Kg wet cell from 120 liter culture) and stored at −20° C. till use.

A part of cell pellet was thawed resuspended in Buffer A (2 L per Kg of cell pellet; 50 mM Tris-HCl, pH8.5, 5 mM sodium azide, 5 mM β-mercaptoethanol, 10 mM imidazole). Cells were lysed by Turrax homogenizer in the presence of 5 mM PMSF and two passes through Big Gaulin at 9,000 psi. The cell extract was heat treated at 90° C. for 30 min (internal temperature reached at 80° C. at the end of the heat treatment) in a water bath and chilled in a ice/water bath till the internal temperature reached below 8° C. Salt was added after heat treatment to the final concentration to 1 M by adding a third of the cell suspension volume of Buffer B (4 M NaCl, 50 mM Tris-HCl, pH8.5, 5 mM sodium azide, 5 mM β-mercaptoethanol, 10 mM imidazole). Cell debris was removed by centrifugation at 4,500 rpm in a H-6000 rotor for 1.5 hr at 4° C. using RC-3B centrifuge. When necessary the supernatant was clarified by a 0.45 μg/m Nalge filter unit. The clarified supernatant was loaded onto a 500 ml Tosof, AF-Chelate-650M column at the flow rate 60 ml/min.

An AF-Chelate-650M column (TosoHaas, 500 ml resin volume) was equilibrated with an equilibration buffer (0.2M NaCl, 50 mM Tris-HCl, pH7.5, 1 mM sodium azide, 2 mM EDTA). The column was washed with 6 column volumes (3000 ml) of Buffer B, followed by 6 column volumes of Buffer C (2.5 M NaCl, 50 mM Tris-HCl, pH8.5, 5 mM sodium azide, 5 mM β-mercaptoethanol, 10 mM imidazole, 40% ethylene glycol) and 10 column volumes of Buffer A. The protein was eluted in a gradient of 10 mM to 150 mM of imidazole (7 column volumes of Buffer A and 0 to 50% of Buffer D; 50 mM Tris-HCl, pH8.5, 5 mM sodium azide, 5 mM β-mercaptoethanol, 300 mM imidazole) at the flow rate of 13 ml/min. Eluate was collected in 20 ml fractions. Fractions containing AccuPrime protein (monitored by absorption at 280 nm) were pooled and diluted 2 fold with Buffer E (50 mM Tris-HCl, pH8.0, 1 mM EDTA, 5 mM β-ME, 5 mM sodium azide).

The diluted fraction pool was loaded to an EMD SO₃-650M column (EMERK, 200 ml bed volume) pre-equilibrated with the equilibration buffer (0.2M NaCl, 50 mM Tris-HCl, pH7.5, 1 mM sodium azide, 2 mM EDTA) at the flow rate of 6.6 ml/min. The column was washed with 10 column volumes (2000 ml) of Buffer E. The protein was eluted with 10 column volumes of 0 to 600 mM NaCl gradient (Buffer E and 0 to 60% of Buffer F; 50 mM Tris-HCl, pH8.0, 1 mM EDTA, 5 mM β-ME, 5 mM sodium azide, 1 M NaCl) in 15 ml fractions at the flow rate of 3.3 ml/min. The fractions containing the protein (fractions with OD₂₈₀ higher than 50% the peak height) were pooled and diluted 3 fold with Buffer E.

The diluted pool of EMD SO₃-650M column fractions was loaded to a High Q column (BioRad, 160 ml bed volume) at the flow rate of 17 ml/min. The column was pre-equilibrated with a mix of Buffer E (90%) and F (10%) (100 mM NaCl, 50 mM Tris-HCl, pH8.0, 1 mM EDTA, 5 mM β-ME, 5 mM sodium azide). The column was washed with 10 column volumes (1.6 L) of the mix of Buffer E and F (9:1) and eluted at the flow rate of 3.3 ml/min with 15 column volumes of a linear gradient of NaCl from 100 to 550 mM (Buffer E and 10 to 55% of Buffer F), collecting 10 ml fractions. After the gradient the column was further eluted with 1.5 column volumes of buffer F to ensure complete elution of the protein. The fractions containing the protein (fractions with OD₂₈₀ higher than 50% the peak height) were pooled. After adding ¼ volume of Storage buffer (100 mM NaCl, 25 mM Tris-HCl, pH7.5, 1 mM EDTA, 1 mM DTT, 50% glycerol) to the pool, and dialyzed against 20 volumes of the Storage buffer with two changes of the buffer overnight.

Protein Assay for Purified AccuPrime Protein

Bradford protein assay was performed using Bio-Rad Protein Assay Dye Reagent Concentrate (Bio-Rad; Part# 500-0006) and lyophilized bovine gamma globulin (Bio-Rad; Part# 500-0005) reconstituted to 1.41 mg/ml as a standard. AccuPrime protein (lot# KP-3) at the concentration of 0.31 mg/ml was used as a stock. Three different measurements at three different dates were made to test reproducibility of the quantitation values.

The same AccuPrime protein stock solution was used to measure concentration using UV absorption at 275 nm. UV spectrum was measured using Beckman Model DU-640 spectrophotometer in a Beckman micro quartz cell (8 mm) from 220 to 320 nm. Absorbance at 320, 310, 275 and 245 nm were read from the spectrum. The absorbance at 320 and 310 nm were used to calculate slope of the baseline, while the absorbance at 275 and 245 nm were used to estimate the extent of nucleic acid contamination. Absorbance at 275 nm was calibrated by subtracting baseline, calculated from the slope of the baseline, using the equation: Abs(275)_{cal}=Abs(275)_{obs}−4.5× (Abs(310)_{obs}−Abs(320)_{obs}), where Abs(275)_{cal} is calibrated absorbance at 275 nm, and Abs(275)_{obs}, Abs(310)_{obs} and Abs(320)_{obs} are measured absorbance at 275, 310 and 320 nm, respectively.

QC Assays for Purified AccuPrime Protein

Endo-nuclease activity. Endo-nuclease assay for a batch of AccuPrime protein was performed using a double-stranded endonuclease assay. Each reaction contained 1 μg of supercoiled φX174 RF DNA and 4 (10×) or 8 (20×) μg of AccuPrime protein in 50 μl of 1× PCR buffer (20 mM Tris-HCl, pH 8.4, 50 mM KCl) including 1.5 mM of MgCl₂. Reaction mix was incubated at 37° C. for 1 hr, and the reaction was terminated by adding 6 μl of 10× BlueJuice (gel loading buffer). The reaction mix was assayed by agarose gel electrophoresis. The electrophoresis was done for 10 μl each of the mixes on a 0.8% horizontal agarose gel and the gel was stained with Ethidium Bromide.

Exo-nuclease activity. 100 pmol of oligonucleotide (36mer; 5′-GGG AGA CGG GGA ATT CGT CGA CGC GTC AGG ACT CTA-3′) was labeled with ³²P at the 5′ end using 10 units of T4 polynucleotide kinase and 10 μCi of [γ-³² P] ATP in 50 μl of 1× PNK exchange buffer. The reaction mix was incubated at 37° C. for 30 min and the reaction was terminated by incubating the mix at 70° C. for 10 min. Unincorporated nucleotides were removed by eluting the reaction mix through Amersham-Pharmacia Micro Spin G-25 column twice following the manufacturers instruction.

40 pmol of the radio-labeled oligonucleotide was incubated with 8 μg (10×) of AccuPrime protein in 100 μl of 1×PCR buffer (20 mM Tris-HCl, pH 8.4, 50 mM KCl) including 1.5 mM of MgCl₂ at 37° C. or 72° C. For samples incubated at 72° C., 20 μl aliquots were taken out at 0, 5, 10, and 30 min, and mixed with 10 μl of 3× formamide sequencing gel loading buffer and stored on ice. The samples were heated at 95° C. for 5 min and 10 μl each was loaded on an 8% polyacrylamide sequencing gel. For samples incubated at 37° C., 20 μl aliquots were taken out at 0, 15, 30, and 60 min, mixed with 1 μl each of 10% SDS and 20 mg/ml Proteinase K (Invitrogen; part# 25530-049), and incubated at 55° C. for 45 min. At the end of reaction, samples were mixed with 10 μl of 3× formamide sequencing gel loading buffer each and heated at 95° C. for 5 min. 10 μl each was loaded on an 8% polyacrylamide sequencing gel. The polyacrylamide gel was dried and autoradiographed using Kodak BioMax MR X-ray film.

Characterization of AccuPrime protein

Single stranded DNA binding. AccuPrime protein affinity for secondary structure of the single stranded DNA was tested with the 84 mer synthetic oligonucleotide KP_PALIN_cont: 5′-CTC CTG GAT CGA CTT CAG TCC GCT GAT GAT TAG ATG TCG TCC TGG ATC GAC TTC ACT CCG CAC CCG CTA CCA ACA ACA GTA CCC-3′. The oligonucleotide was radiolabeled at the 5′ end in the same manner as the 5′ (ss) substrate for the exo-nuclease activity assay above with the oligonucleotide concentration at 5 μM.

The protein-oligonucleotide binding was performed in 50 μl of 1× PCR buffer including 1.5 mM MgCl₂ with the protein concentrations varying from 0 to 40 nM with an increment in step of 10 nM at the oligonucleotide concentration at 20 nM. The reaction mixes were incubated at 70° C. for 5 min and loaded on a 6% non-denaturing horizontal polyacrylamide gel with the current on. The electrophoresis was done at 100V for 1 hr. The gel was dried and autoradiographed on Kodak BioMax MR X-ray film.

Effect on Tag DNA polymerase unit activity. To see the effect of AccuPrime protein on Taq DNA polymerase activity at the elongation phase, the incorporation rate of radiolabeled nucleotides was measured using nicked salmon testes DNA or pre-primed M13mp19 circular single stranded DNA in the presence of various concentrations of Taq DNA polymerase and AccuPrime protein. The nucleotide incorporation into acid-insoluble fraction was measured by spotting a fraction of reaction to GF/C filter, washing the filter with TCA solution, and counting the amount of radioactivity decay in the filter using a scintillation counter.

Brief descriptions of assays are as following. For a standard unit assay in the presence of AccuPrime protein, each of serial dilutions of Taq DNA polymerase to 0.0083, 0.0125, 0.025 units was added to a set of 50 μl reactions containing 0.1, 0.2, 0.4, 1 or 3.2 μg of AccuPrime protein. Each reaction contained 0.5 μg/μl of nicked salmon tested DNA, 0.2 mM each of nucleotides in 1× Taq unit assay buffer (25 mM TAPS, pH 9.3, 50 mM KCl, 2 mM MgCl2, 1 mM DTT and 1 to 2 μCi [α-³²P] dCTP in the final volume of 50 μl per reaction. The reaction was initiated upon addition of Taq polymerase and transfer to heating block equilibrated to 72° C. The reaction was continued for 10 min and terminated by adding 10 μl of 0.5 M EDTA to each of the 50 μl reactions on ice. 40 μl each of the reactions was spotted onto a GF/C filter for TCA precipitation. In a similar experiment, nucleotide incorporation rate was measured by spotting 10 μl aliquots of a reaction at several time points, 0, 5, 10 and 30 min, to separate GF/C filters during incubation. Incubation temperature for this experiment was varied from 55 to 74° C. with 5° increment to see the temperature effect on AccuPrime protein function. The AccuPrime protein concentration, when present, was 0.1 μg per 50 μl reaction.

For more defined mechanistic studies, pre-primed single stranded circular M13mp19 DNA was used in place of nicked salmon testes DNA as template. The primer, M13mp19_{—}1442L30, used in this study was designed to anneal to coordinate 1442 of the (+) strand of M13mp19 DNA and has the sequence: 5′-GCC GAC AAT GAC AAC AAC CAT CGC CCA CGC-3′.

The primer was mixed with the template at the 2 to 10 folds molar excess to the template in TE buffer, heated at 95° C. for 5 min, and slow-cooled to room temp for 30 min. For a 50 μl reaction 0.4 to 3.2 pmole template was used with 0.125 to 0.5 units of Taq DNA polymerase. However, due to a lower substrate:polymerase ratio, the reaction rate would be easily saturated by a slight increase of the enzyme in a given concentration of the template. The total amount of polymerase should be below a saturation level which should be empirically determined at a given template concentration. For instance, 0.125 units of polymerase were below saturation with 0.4 pmole template, while 0.5 units of the enzyme was saturating with 3.2 pmole of template. The AccuPrime protein concentration, when present, was 50 ng per 50 μl reaction. Incubation temperature was set at 70° C., but all other conditions were the same as above. The nucleotide incorporation rate was measured by spotting 10 μl aliquots of a reaction at several time points, 0, 5, 10 and 30 min, to separate GF/C filters during incubation.

TCA precipitation for the samples on GF/C filters were performed following the standard protocol, 30001.SOP. The filters were washed first in 10% TCA solution containing 1% sodium pyrophosphate for 15 min, and in 5% TCA for 10 min three times, followed by wash in 95% ethanol for 10 min. The filters were dried under a heat lamp for 5 to 10 min and the radioactivity decay rate was measure in ScintiSafe Econo 1 scintillation cocktail (Fisher Scientific, part# SX20-5) using a Beckman scintillation counter (Model# LS 3801).

Effect on elongation activity of Tag DNA polymerase. The M13mp19 primer, M13 mp19_{—}1442L30, was radiolabeled using T4 polynucleotide kinase and [γ-³²P] ATP as above, and annealed to single stranded circular M13mp 19 DNA at the primer:template molar ratio of 10:1.

The elongation reaction was set in the final volume of 350 μl, equivalent of 7×50 μl reactions. During incubation at 70° C., 50 μl aliquots were taken out at 30 sec intervals up to 2 min and mixed with 10 μl of 0.5M EDTA to terminate the elongation. Each 50 μl aliquot contained 0.18 pmole of the pre-primed template, 10 units of Taq DNA polymerase, and 0, 50 or 100 ng of AccuPrime protein or 100 ng of MthSSB (AccuPrime protein homologue from M. thermoautotropicum) in 1× Taq polymerase unit assay buffer. To each of the 60 μl aliquot, 7 μl of 3M sodium acetate and 175 μl of 95% ethanol were added. DNA was precipitated at −20° C. for 2 hrs and pelleted in a micro-centrifuge at the maximum speed for 15 min at 4° C. After the supernatant was removed, the pellet was air-dried and resuspended in 20 μl of alkaline gel loading buffer (10 mM NaOH, 0.1% bromophenol blue, and 10% glycerol).

1% agarose gel (11×14 cm) was made in 50 mM NaCl, 1 mM EDTA solution and, after solidified, soaked in 30 mM NaOH, 1 mM EDTA solution at room temperature for at least 2 hrs. 8 μl each of the samples was loaded on the gel, and electrophoresis done at 95 volt for 1 hr. The gel was dried under vacuum for 30 min without heat and further dried at 50° C. under vacuum for another hour. The gel was autoradiographed onto a phospho-imager plate from Molecular Dynamics, and the image was processed using ImageQuant ver 3.3 program. Densitometry was performed with NIH Image ver 1.61 program from the image file converted to TIFF format in the ImageQuant program.

Stability of AccuPrime Protein in AccuPrime Formulation

Accelerated stability assay. Accelerated stability assay is based on assumption that an elevated temperature would thermodynamically accelerate the rate of a reaction, and that deterioration (inactivation, denaturation or degradation) of a protein is a reaction from the thermodynamic point of view. Therefore, incubating a protein solution at a higher temperature for a certain period of time would mimic an effect of a longer period of storage at a lower temperature. The extent of the acceleration was estimated using Arrhenius equation: k=A e^{[-Ea/R T]} where k is the rate constant, A is a constant, Eₐ is the activation energy, R is the universal gas constant 8.314×10⁻³ kJ mol⁻¹K⁻¹, and T is the temperature (in degrees Kelvin).

All four different AccuPrime Taq DNA polymerase formulations were tested at 37° C. and 45° C. for 7 and 4 days, respectively, which were equivalent to 1 yr of storage at −20° C. The four different formulations are: 10× reaction mix for genomics with AccuPrime protein concentration at 4 μg/50 μl (10× AccuPrime Taq PCR Reaction Mix II); 10× reaction mix for cDNA with the protein concentration at 0.5 μg/50 μl (10× AccuPrime Taq PCR Reaction Mix I); 2× Supermix for genomics with the protein concentration at 0.8 μg/50 μl (2× AccuPrime Taq PCR SuperMix II); and 2× Supermix for cDNA with the protein concentration at 0.1 μg/50 μl (2× AccuPrime Taq PCR SuperMix 1). For 10× reaction mix formulation for genomics, two different batches were made: one with nucleotides; and the other without nucleotide.

After the period of incubation, the reaction mix (or Supermix) was tested for its function using PCR at 1× strength. For the functional assay, a primer set was selected for its difficulty in its PCR in obtaining specific product with other Taq DNA polymerases. The sequences of the Rhod_{—}626 primer set primers are: forward primer (Rhod_{—}147F) 5′-AGG AGC TTA GGA GGG GGA GGT-3′; reverse primer (Rhod_{—}773R) 5′-CAT TGA CAG GAC AGG AGA AGG GA-3′.

The non-specific bands were to be eliminated by functional AccuPrime Taq, leaving only the enhanced specific band of 626 bp in length. PCR reaction was carried out in a standard manner using 20 ng of K562 genotyping DNA as template and 0.2 μM each of the primers in 50 μl of 1× PCR buffer including 1.5 mM MgCl₂. PCR incubation was set with 94° C. pre-incubation for 2 min, followed by 35 cycles of 94° C. for 15 see, 58° C. for 30 see and 68° C. for 1 min. The PCR products were analyzed on a 1% horizontal agarose gel.

Real-time stability assay. Real-time stability assay was performed similarly to the accelerated stability assay with a few exceptions. This time all the formulation contained nucleotides. The lot for 10× reaction mix formulation was divided to two batches to include one batch without glycerol. Incubation was done at three different temperatures, −20, 4 and 22° C.

One more set of primers, in addition to the primer set above, was used for PCR functional assay of the reaction mixes for genomic and cDNA templates, respectively. For AccuPrime Taq Reaction Mix I and AccuPrime Taq SuperMix I, pUC19₁₃ 2.7 primers were used: forward primer: (pUC19_{—}2182F) 5′-TCA ACC AAT TCA TCC TGA GAA TAG T-3′; reverse primer (pUC19_{—}2177R) 5′-TCA CCA GTC ACA GAA AAG CAT CTT AC-3′. For AccPrime Taq Reaction Mix II and AccuPrime Taq SuperMix II, the Rhod_{—}626 primer set was used.

The criterion for the PCR functional assay was same as above in that a functional reaction mix would suppress non-specific bands while enhance specific band of 4.4 Kb, 626 bp and 2.7 Kb in length for p53_{—}4.4, Rhod_{—}626 and pUC19_{—}2.7 primer sets, respectively. PCR reaction was carried out in a standard manner using 20 ng of K562 genotyping DNA as genomic template or 200 fg of pUC19 as cDNA template and 0.2 μM each of the primers in 50 μ1 of 1×PCR buffer. PCR incubation was set the same above except annealing temperature and elongation period, which were 60° C. and 4 min for p53_{—}4.4, 58° C. and 1 min for Rhod_{—}626, and 54° C. and 2.5 min for pUC19_{—}2.7, respectively. The PCR products were analyed by 0.8-1% agarose gel electrophoresis.

Post-PCR Assay for AccuPrime Taq DNA Polymerase Amplification

During the a testing, a several questions were raised regarding the end product of PCR amplification using AccuPrime Taq DNA polymerase. The questions pertain to the compatibility of the amplification products with downstream applications such as TOPO TA cloning and RFLP assays. To address such concerns, we performed PCR amplification and assayed the amplification product for its compatibility with such downstream applications.

TOPO TA cloning. Two separate amplicons from pUC19 were selected for their general usage and GC-richness. The first amplicon (multi-cloning site) was selected for frequency of its use. The commercial M13/pUC Amplification primers were used for the PCR reaction: forward primer (LTI, 18431-015) 5′-CCG AGT CAC GAC GTT GTA AAA CG-3′; reverse primer (LTI, 18432-013) 5′-AGC GGA TAA CAA TTT CAC ACA GG-3′. The second amplicon was selected for its GC-richness (62% GC content): forward primer (pUC19_{—}606f) 5′-CCA GTC GGG AAA CCT GTC GT-3′; reverse primer (pUC19_{—}745r): 5′-ACC GCC TTT GAG TGA GCT GA-3′. The amplicons were 136 and 159 bp long, respectively.

PCR reactions were prepared in 50 μl reaction volumes containing 1× PCR buffer (20 mM Tris-HCl, pH 8.4, 50 mM KCl), 1.5 mM MgCl₂, and 0.2 μM of each primer. The concentration of each of four deoxynucleoside triphosphate (dNTPs) was 0.2 mM. Template concentration varied from 100 pg (for plasmids and cDNA) to 100 ng (genomic DNA) depending on the application. Two units of AccuPrime Taq DNA polymerase and 2 units of Platinum Taq DNA polymerase as control were used in a typical 50 μl reaction. Thermocycling was conducted using the Perkin Elmer GeneAmp PCR System 2400:

| | |
|---|---|
| 94° C. | 2 minutes |
| 35 cycles of | |
| 94° C. | 15 seconds |
| 58° C. | 30 seconds |
| 68° C. | 1 min |
| Hold at 4° C. | |

Following the completion of thermocycling, PCR amplification products were analyzed on 2% agarose gel electrophoresis to make sure that the right sizes of amplicons were amplified. The PCR products then were used for TOPO TA cloning according to the manufacturer's instruction. The resulting clones were purified and sequenced using ABI automatic sequencer.

Restriction Endonuclease Digestion. For RFLP assay, the p53 primer set with its amplicon size of 220 bp was used with 50 to 200 ng of genomic DNA (K562) as template. PCR was performed similarly as above including the Platinum Taq control reaction:

| | |
|---|---|
| 94° C. | 2 minutes |
| 35 cycles of | |
| 94° C. | 15 seconds |
| 55° C. | 30 seconds |
| 68° C. | 1 min |

After PCR amplification, the product, either 5 or 10 μl for each reaction, was digested with 10 units of ScaI or PvuII in 20 μl digestion reaction at 37° C. for 2 hr in appropriate buffers as recommended by the manufacturers. The digestion products were assayed on 2% agarose gel electrophoresis.

PCR Application Development for AccuPrime Taq DNA Polymerase

Standard PCR reactions. Unless otherwise indicated, all the PCR reactions were run following a standard protocol. PCR reactions were prepared in 50 μl reaction volumes containing 1× PCR buffer (20 mM Tris-HCl, pH 8.4, 50 mM KCl), 1.5 mM MgCl₂, and 0.2 μM of each primer. The concentration of each of four deoxynucleoside triphosphate (dNTPs) was 0.2 mM. Template concentration varied from 100 pg (for plasmids and cDNA) to 100 ng (genomic DNA) depending on the application. Two units of AccuPrime Taq DNA polymerase were used in a typical 50 μl reaction. Primer sets used in development of AccuPrime Taq DNA polymerase system and its applications are listed in Table 2.

TABLE 2

| | |
|---|---|
| p32D9 149 bp | 5′ 3′ |
| Forward primer: | ATC CCC CAC CCC CGC ACC |
| Reverse primer: | GGG CGC GAG ATG GGC TGC |
| Pr1.2 235 bp | 5′ 3′ |
| Forward primer: | TTG GAG GGG TGG GTG AGT CAA G |
| Reverse primer: | GGA GGG GTG GGG GTT AAT GGT TA |
| Pr1.3 265 bp | 5′ 3′ |
| Forward primer: | GCA TCT GGG GCC TGG GAT TTA G |
| Reverse primer: | TAC AAG GCA GGC ATC ATG ACT CAC G |
| p53 gene 504 bp | 5′ 3′ |
| Forward primer: | TGC CGT CCC AAG CAA TGG ATT T |
| Reverse primer: | CAG GAG AGA TGC TGA GGG TGT GGA |
| c-myc gene 822 bp | 5′ 3′ |
| Forward primer: | CGG TCC ACA AGC TCT CCA CTT G |
| Reverse primer: | CTG TTT GAC AAA CCG CAT CCT TGc- |
| myc gene 1069 bp | 5′ 3′ |
| Forward primer: | GGT TTT CGG GGC TTT ATC TAA CTC |
| Reverse primer: | GCC TAC CCA ACA CCA CGT CCT |
| p53 gene 1587 bp | 5′ 3′ |
| Forward primer: | GCT GCC GTG TTC CAG TTG CTT TAT C |
| Reverse primer: | GCA GCT CGT GGT GAG GCT CCC |
| p53 gene 1996 bp | 5′ 3′ |
| Forward primer: | CCT TGG CTT TTG AAA ATA AGC TCC TGA |
| Reverse primer: | GCA GCT CGT GGT GAG GCT CCC |
| p53 gene 2108 bp | 5′ 3′ |
| Forward primer: | GCA GAG ACC TGT GGG AAG CGA AAA |
| Reverse primer: | GAG AGC TGT GGC AAG CAG GGG A |
| Rhodopsin gene 3047 bp | 5′ 3′ |
| Forward primer: | GCC CTA ACT TCT ACG TGC CCT TCT |
| Reverse primer: | AGG CTT CCA GCG CAC GTC ATT |
| p53 gene 4356 bp | 5′ 3′ |
| Forward primer: | CCC CTC CTG GCC CCT GTC AT |
| Reverse primer: | GTT AGA TGA CTT TGC CCA ACT GTA GGG |

Thermocycling was conducted using either the Perkin Elmer GeneAmp PCR System 9600 or the Perkin Elmer GeneAmp PCR System 2400.

Standard PCR Program:

| | |
|---|---|
| 94° C. | 2 minutes |
| 35 cycles of | |
| 94° C. | 15 seconds |
| 55° C.-60° C. | 30 seconds (5 degrees below Tm) |
| 68° C. | 1 min/kb |
| Hold at 4° C. | |

Following the completion of thermocycling, PCR amplification products were mixed with 5 ml of 10× BlueJuice and aliquot (20%, or 10 μl, of total reaction volume per each lane) were analyzed on 0.8%-1.5% agarose gel electrophoresis with an ethidium bromide concentration of 0.5 μg/ml premixed in 0.5×TBE. The resulting gels were analyzed visually for specificity and yield among different samples.

Miniaturization. PCR reactions were prepared for 10 μl and 25 μl reactions using proportionally reduced volumes of 10×PCR buffer, 50 mM MgCl₂, and 10 μM Primer. Final standard concentrations for each component were 1×, 1.5 mM, and 0.2 μM, respectively. Deionized water was used to QS to the appropriate volumes. A 20 ng/μl amount and concentration of K562 human genotyping DNA template remained constant throughout all experiments. Thermocycling was conducted using either the Perkin Elmer GeneAmp PCR System 9600 or the Perkin Elmer GeneAmp PCR System 2400.

Titrations with dNTP and primer were conducted with five points 0.1 mM, 0.15 mM, 0.2 mM, 0.3 mM and 0.4 mM final concentration. In addition, a comparison in enzyme units was made between 0.5 units and 1 unit. PCR reactions were prepared at room temperature in attempts to decrease the efficiencies of the polymerases, and enhance the advantages of AccuPrime Taq.

PCR program for dNTP and Primer Titration:

| | |
|---|---|
| 25° C. | 60 minutes |
| 94° C. | 2 minutes |
| 35 cycles of | |
| 94° C. | 15 seconds |
| 55° C.-60° C. | 30 seconds (5 degrees below Tm) |
| 68° C. | 1 min/kb |
| Hold at 4° C. | |

To determine the optimal enzyme units required, titrations were focused on six different amounts: 0.2 units, 0.4 units, 0.6 units, 1 unit, 1.5 units, and 2 units.

PCR Program for Enzyme Titration:

| | |
|---|---|
| 94° C. | 2 minutes |
| 35 cycles of | |
| 94° C. | 15 seconds |
| 55° C.-60° C. | 30 seconds (5 degrees below Tm) |
| 68° C. | 1 min/kb |
| Hold at 4° C. | |

To optimize the concentration of Single Stranded Binding protein in miniaturized reactions titration were conducted using 80 ng, 100 ng, and 120 ng for 10 μl reactions and 160 ng, 200 ng, 240 ng for 20-25 μl reactions. In addition, 1 unit of enzyme was used throughout.

PCR Program for SSB Titration:

| | |
|---|---|
| 94° C. | 2 minutes |
| 35 cycles of | |
| 94° C. | 15 seconds |
| 55° C.-60° C. | 30 seconds (5 degrees below Tm) |
| 68° C. | 1 min/kb |
| Hold at 4° C. | |

Following the completion of thermocycling, PCR products were analyzed on 1.2%-1.5% agarose gel electrophoresis with an ethidium bromide concentration of 0.5 μg/ml premixed in 0.5×TBE. Comparisons were made visually between specificity and yield for the different samples.

Difficult templates. Initial experiments were conducted by varying only the annealing temperatures in the range of 55-60° C. Standard concentrations and amounts of PCR buffer, MgCl₂, dNTP, and Primer were used. With the next series of experiments we tried substituting Hi-Fi Buffer and MgSO₄ for 10×PCR buffer and MgCl₂. In the third series of experiments we made use of PCRx enhancer, a solution specially designed to improve difficult templates. Titrations of PCRx enhancer solution (available from Invitrogen Corp.) were applied covering a range of zero to 3×.

PCR Program for Difficult Template:

| | |
|---|---|
| 94° C. | 2 minutes |
| 35 cycles of | |
| 94° C. | 15 seconds |
| 55° C.-60° C. | 30 seconds (5 degrees below Tm) |
| 68° C. | 1 min/kb |
| Hold at 4° C. | |

Multiplex PCR. Random designs of primer sets from different genes were selected for multiplex PCR. To determine the optimal conditions, titrations were conducted involving all practical aspects of a standard PCR reaction such as:
a) DNA template—using 100 ng, 200 ng, and 500 ng.
b) Enzyme units—with 2 units, 5 units, and 10 units.
c) dNTP—focusing on 0.1 mM, 0.2 mM, and 0.4 mM final concentrations.
d) MgCl₂—centering on, 1.2, 1.5, 1.8, 2, and 2.5 mM final concentrations.
e) Single Stranded Binding Protein concentration—200, 400, 600, and 800 ng.

PCR reactions were prepared on ice in the standard format using 100 ng of K562 genotyping DNA as a template and 2-5 units of enzyme in addition to the obvious substitution of each of the variables as outlined above. The primer sets used in multiplex PCR are listed in Table 3.

TABLE 3

| | |
|---|---|
| #1 | |
| Tms1-44 | 5′ 3′ |
| Forward primer: | GGC TGG AGT GCA GTG GTG CAA T |
| Reverse primer: | GGC AGA GGC TAC AGT GAG CCA A |
| #2 | |
| Thal-57 | 5′ 3′ |
| Forward primer: | GGG CAG AGC CAT CTA TTG CTT ACA |
| Reverse primer: | GGT TGC TAG TGA ACA CAG TTG TGT CA |
| #3 | |
| Hba2-67 | 5′ 3′ |
| Forward primer: | GCA CTC TTC TGG TCC CCA CAG A |
| Reverse primer: | TTG GTC TTG TCG GCA GGA GAC A |
| #4 | |
| Rgr-74 | 5′ 3′ |
| Forward primer: | CCC ACG ATC AAT GCC ATC AAC T |
| Reverse primer: | CGG TGA GAG GCA CTG CCA GAT T |
| #5 | |
| B-glo-thal-84 | 5′ 3′ |
| Forward primer: | GCT CGC TTT CTT GCT GTC CAA T |
| Reverse primer: | GCC CTT CAT AAT ATC CCC CAG TTT |
| #6 | |
| c-myc-100 | 5′ 3′ |
| Forward primer: | GTC CTT CCC CCG CTG GAA AC |
| Reverse primer: | GCA GCA GAG ATC ATC GCG CC |
| #7 | |
| Zip-116 | 5′ 3′ |
| Forward primer: | GTG GGG GTG CTG GGA GTT TGT |
| Reverse primer: | TCG GAC AGA AAC ATG GGT CTG AA |
| #8 | |
| Csh1-135 | 5′ 3′ |
| Forward primer: | GGT GCT CAG AAC CCC CAC AAT C |
| Reverse primer: | CCT ACC GAC CCC ATT CCA CTC T |
| #9 | |
| Sub-153 | 5′ 3′ |
| Forward primer: | CAC AGA TTT CCA AGG ATG CGC TG |
| Reverse primer: | CGT GCT CTG TTC CAG ACT TG |
| #10 | |
| Svmt-170 | 5′ 3′ |
| Forward primer: | CGT CTG GCG ATT GCT CCA AAT G |
| Reverse primer: | GGG CAG TTG TGA TCC ATG AGA A |
| #11 | |
| Olf-183 | 5′ 3′ |
| Forward primer: | GGC TTG CAC CAG CTT AGG AAA G |
| Reverse primer: | CGT TAG GCA TAA TCA GTG GGA TAG T |
| #12 | |
| P53-193 | 5′ 3′ |
| Forward primer: | GCC TCT GAT TCC TCA CTG ATT GCT CT |
| Reverse primer: | TGT CAA CCA CCC TTA ACC CCT CC |
| #13 | |
| Pr1.2-237 | 5′ 3′ |
| Forward primer: | TTG GAG GGG TGG GTG AGT CAA G |
| Reverse primer: | GGA GGG GTG GGG GTT AAT GGT TA |
| #14 | |
| Hmk-243 | 5′ 3′ |
| Forward primer: | GGA ACA AGA CAC GGC TGG GTT |
| Reverse primer: | AGC AAG GCA GGG CAG GCA A |
| #15 | |
| Rhod-273 | 5′ 3′ |
| Forward primer: | CGG TCC CAT TCT CAG GGA ATC T |
| Reverse primer: | GCC CAG AGG AAG AAG AAG GAA A |
| #16 | |
| Caaf1-300 | 5′ 3′ |
| Forward primer: | GCC CCC ACC CAG GTT GGT TTC TA |
| Reverse primer: | ATG CCT TCA TCT GGC TCA GTG A |
| #17 | |
| P-450 B-319 | 5′ 3′ |
| Forward primer: | GCT CAG CAT GGT GGT GGC ATA A |
| Reverse primer: | CCT CAT ACC TTC CCC CCC ATT |
| #18 | |
| S-100-360 | 5′ 3′ |
| Forward primer: | GAC TAC TCT AGC GAC TGT CCA TCT C |
| Reverse primer: | GAC AGC CAC CAG ATC CAA TC |
| #19 | |
| B-cone-432 | 5′ 3′ |
| Forward primer: | GGC AGC TTT CAT GGG CAC TGT |
| Reverse primer: | GAC AGG GCT GGA CTG ACA TTT G |
| #20 | |
| Hbg-469 | 5′ 3′ |
| Forward primer: | CTG CTG AAA GAG ATG CGG TGG |
| Reverse primer: | AGG AAA ACA GCC CAA GGG ACA G |

Standard program for multiplex PCR reactions

| | |
|---|---|
| 94° C. | 2 minutes |
| 35 cycles | |
| 94° C. | 15 seconds |
| 60° C. | 30 seconds (5 degrees below Tm) |
| 68° C. | 1 min/kb |
| Hold at 4° C. | |

The PCR products were then analyzed on a 3% horizontal agarose gel with an ethidium bromide concentration of 0.5 μg/ml premixed in 0.5×TBE. Comparisons were made visually for specificity and yield between the different samples.

High throughout PCR. Accuprime Taq DNA polymerase was compared with Platinum™ Taq DNA polymerase (Invitrogen Corp.) to examine for improvement in high throughput screening. Standard PCR was performed for 18 cycles of amplification using 2 Units of Accuprime Taq DNA polymerase and 2 Units of Platinum Taq DNA polymerase.

Transformed cells plated on X-gal/IPTG/Amp plates containing the pUC19 plasmid DNA insert were used as plasmid template for high throughput screening. Mutant colonies were selected with a sterile pipette tip and mixed in the standard PCR reactions. PCR cycling parameters were 94° C. for 2 min, followed by 18 cycles of 94° C. for 15 s, 55° C. for 30 s, and 68° C. for 3 min. PCR product was analyzed by agarose gel electrophoresis.

rpsL Fidelity Assay

Fidelity assay was performed based on streptomycin resistance of rpsL mutation exhibits (Lackovich et al., 2001; Fujii et al., 1999). Briefly, pMOL 21 plasmid DNA (4 kb), containing the ampicillin (Ap′) and (rpsL) genes, was linearized with Sca I and standard PCR was performed on the linearized product using biotinylated primers. Amplification was completed using 2 units of AccuPrime Taq DNA polymerase. Template DNA was 1 ng for 25 cycles of amplification. PCR cycling parameters were 94° C. for 2 min, followed by 25 cycles of 94° C. for 15 s, 58° C. for 30 s, and 68° C. for 5 min. PCR product was streptavidin-magnetic-bead-purified to ascertain linearity. Purified PCR product was analyzed on an agarose gel, and DNA concentration and template doubling was estimated. The purified DNA was ligated with T4 DNA ligase and transformed into MF101 competent cells. Cells were plated on ampicillin plates to determine the total number of transformed cells. Cells were plated on ampicillin and streptomycin plates to determine the total number of rpsL mutants. Mutation frequency was determined by dividing the total number of mutations by the total number of transformed cells. The error rate was determined by dividing the mutation frequency by 130 (the number of amino acids that cause phenotypic changes for rpsL) and the template doubling.

### RESULTS AND DISCUSSION

Purification of AccuPrime Protein

Due to the toxicity of the protein expressed, exerted most likely by interfering with replication and recombination intermediates, even at the basal level of lac promoter used in the construct, the cells grew extremely slow (1.5 hr doubling time). Probably due to the toxicity, the yield of the purified protein was very low and highly dependent on the media used (Table 4). Though not tested, a slow growing condition would enhance the yield.

TABLE 4

| | Volume | Cell | | Protein amount |
|---|---|---|---|---|
| | used | mass | | (purity) |
| Media | (liter) | (gram) | Procedure | & comments |
| LB | 12 | 40 | Ni-NTA agarose, | 6 mg (95%) |
| | | | ssDNA agarose & | Should've been |
| | | | mono Q | 12 mg, since |
| | | | | approx. 50% cell |
| | | | | mass used in |
| | | | | optimization |
| | | | | trials. |
| Terrific | 10 | 50 | Protease | 57 mg (90%) |
| Broth | | | inhibitors, | |
| | | | Ni-NTA agarose, | |
| | | | ssDNA agarose & | |
| | | | mono Q | |
| Magnificent | 6 | 40 | Protease | ˜16 mg (90%) |
| Broth | | | inhibitors, | Heavy DNA |
| | | | Ni-NTA agarose & | contamination. |
| | | | mono Q | Unknown amount |
| | | | | lost due to cell |
| | | | | lysis during |
| | | | | thawing, leak in |
| | | | | dialysis tubing |
| | | | | and freak |
| | | | | accident during |
| | | | | mono Q run. |

The other reason for the low yield could be found in active proteolysis of the protein during purification. As an evidence for the proteolysis, inclusion of the protease inhibitor cocktail greatly enhanced the yield. When cells were grown in Magnificent Broth (MacConnell Research), spontaneous lysis of the cells was observed while thawing the frozen cell pellet. Since the protease inhibitors were not introduced while thawing, proteases were able to digest a considerable fraction of the protein as evidenced by the appearance of a second smaller band below the major full-length protein band in a SDS-PAGE. This could have been prevented by thawing under lysis buffer with the protease inhibitors.

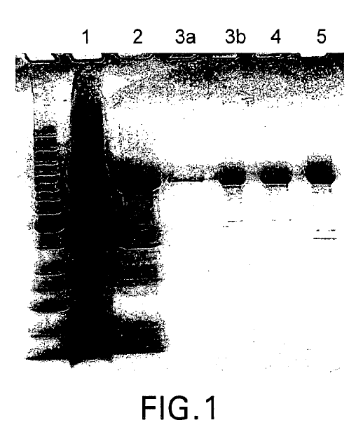

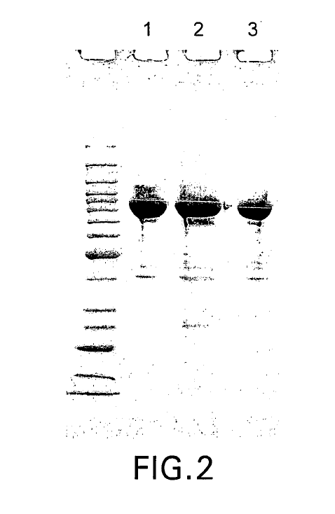

The protocol was not optimized in preventing loss of the protein. The protocol I was following was developed by the UC Davis group where an optimum purification scheme might not have factored in the maximum quantity of the protein per prep (FIG. 1). The procedure in question is the ssDNA column chromatography step. While the protein was eluted in a broad peak within 2 column volumes from the ssDNA agarose column, a considerable amount of the protein was eluted as a long trail following the main peak. The loss by cutting the tail off the peak was estimated to be up to 50% of potential amount. Yet, ssDNA column did not improve the purity enough to warrant the loss (FIG. 2).

Protein Assay for AccuPrime Protein

Table 5 shows three independent measurements each of the two protein assays using a single protein stock solution. The standard deviation for Bradford assay was higher than 2 times that of the UV absorption. The value indicates that one in three measurements the protein concentration determined by the Bradford assay would be off by more than 15% from its real concentration, compared to about 6% from the UV absorption method. The results clearly show the inherent problem associated with protein assay methods using chromogenic dyes, such as Bradford assay. While UV absorption is an intensive property of the solution, Bradford assay measures an extensive property of the solution (the volume of the sample solution added to the dye solution, in addition to the concentration, determines the outcome). Another variant of the Bradford assay is the necessary standard curve that introduces yet another set of manipulation errors.

TABLE 5

| Assay | Bradford | UV absorption |
|---|---|---|
| First | 0.32 | 0.31 |
| Second | 0.44 | 0.36 |
| Third | 0.34 | 0.32 |
| Deviation | 0.36 ± 0.052 | 0.33 ± 0.021 |

QC Assays for AccuPrime Protein

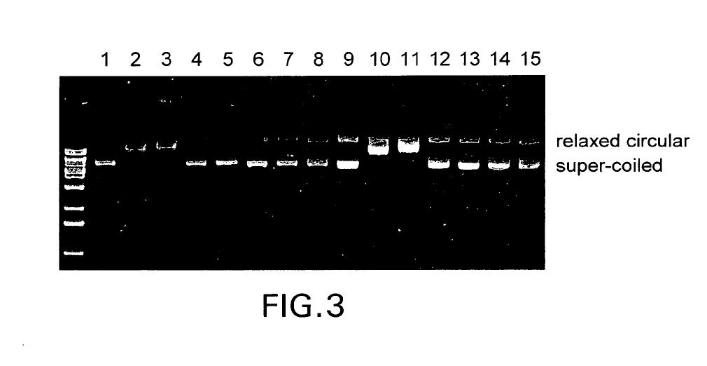

Endo-nuclease activity. No detectable endonuclease activity was found in the AccuPrime protein prep at the protein concentration up to 20× of that recommended for PCR reaction using a double stranded, super-coiled substrate (FIG. 3). After hour incubation at 37° C., the ratio of band intensities for relaxed and supercoiled DNA did not change from that of control that was incubated under identical condition except the absence of the protein. This result indicates that not even a nicking activity was found in the prep.

Exo-nuclease activity. Exonuclease activity was tested in two different temperatures under otherwise an identical condition: at 37° C. and 72° C. While the exonuclease activity stemming from E. coli during purification was checked at the lower temperature incubation, an intrinsic exonuclease activity the protein might have was checked at the higher temperature. The exonuclease activity assay in both cases was with 5′ radiolabeled single stranded oligonucleotide. Both of the reactions were aliquoted and terminated at several time points during the time course to check progressions of the reaction, and the products were analyzed in a denaturing polyacrylamide gel electrophoresis. Autoradiogram of the gel would be able to distinguish whether the solution have 3′ or 5′ exonuclease activity or not. A 3′ exo activity would decrease the length of the labeled nucleotide gradually, a 5′ exo activity, on the other hand, would decrease the band intensity of the full length oligonucleotide band with concurrent appearance of a band corresponding to radiolabeled mononucleotide without intermediate bands in between.

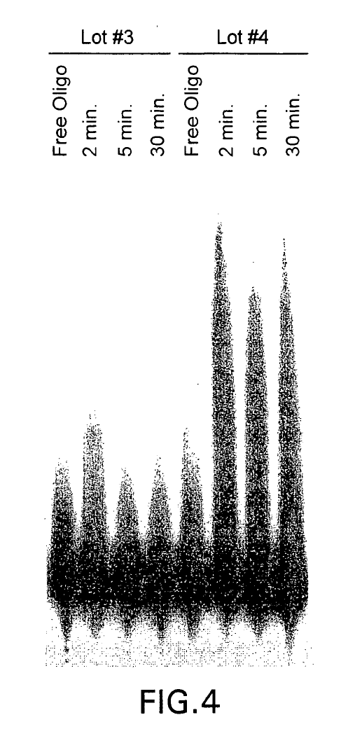

The gel (FIG. 4) showed only a single band corresponding to the radiolabeled full-length oligonucleotide without even reduction in band intensity up to 30 min at 72° C. or to 60 min at 37° C. One peculiar observation, however, was the necessity of proteinase K treatment for the samples incubated with the protein, due to the mobility shift of the band presumably because of the protein binding that survived heating at 95° C. for 5 min in the presence of 30% formamide. This fact along with data from purification indicates a very strong binding of the protein to the oligonucleotide, which might protect the oligonucleotide from any contaminating exonuclease activity especially if the oligonucleotide is short enough.

Characterization of AccuPrime protein

Single stranded DNA binding. The binding affinity of AccuPrime protein to single stranded DNA (ssDNA) was measured using electrophoretic mobility shift assay (EMSA) on 6% horizontal polyacrylamide gel in TBE. The ssDNA molecules used in the assay were synthetic 86-mer oligonucleotides. The 5′ radiolabeled oligonucleotides were incubated with increasing amounts of the protein in 1×PCR buffer (20 mM Tris-HCl, pH 8.4, 50 mM KCl, 1.5 mM MgCl₂) at 70° C. for 5 min, and an aliquot of the reaction mix was loaded on the gel with currents on. The electrophoresis was continued for 1 hr and the gel was autoradiographed after dried.

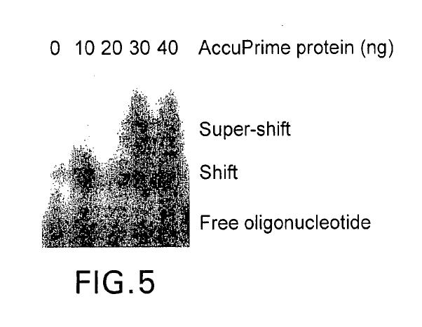

The gel (FIG. 5) showed the mobility of the oligonucleotide shifted almost stoichiometrically with the amount of the protein in the reaction mix, indicating a strong binding as expected. What was unexpected was the presence of a super-shifted band (a band showing higher mobility shift shown above the shifted band). The usual explanation for the super-shifted band is an additional protein binding to protein-DNA complex making further retardation of already retarded protein-DNA complex band.

With the oligonucleotide, the pattern of the mobility shift as the protein concentration increased was that of protein binding to DNA with rather negative cooperativity. Typical negative cooperativity would not show any super-shift even at the highest protein concentration. However, a protein with a rather weak negative cooperativity would allow second protein binding to a same DNA molecule only when the protein concentration was high enough. Such was the observation with the control oligonucleotide.

Effect on Taq polymerase unit activity. It was believed that SSB protein would help DNA polymerases in the elongation phase by removing secondary structures from the template DNA. However, in PCR where the reaction takes place at an high temperature, the secondary structure would play a dismal, if any, role in elongation of the polymerase, so that any enhancement of polymerase activity through such mechanism would be marginal. Nonetheless, it provides a good starting point in exploring the mechanism of AccuPrime protein in enhancement of Taq DNA polymerase activity.

The unit activity assay for Taq DNA polymerase was performed using two different templates: First, nicked and gapped salmon testes DNA where the primed sites would be molar excess to that of the polymerase would provide information regarding the initiation step of the elongation. The initiation step involves recruiting all the necessary components of elongation at the primed site including the polymerase. Second, a pre-primed circular single strand DNA template where a sequence specific primer was annealed to a long circular ssDNA template would provide information about the rate of the elongation.

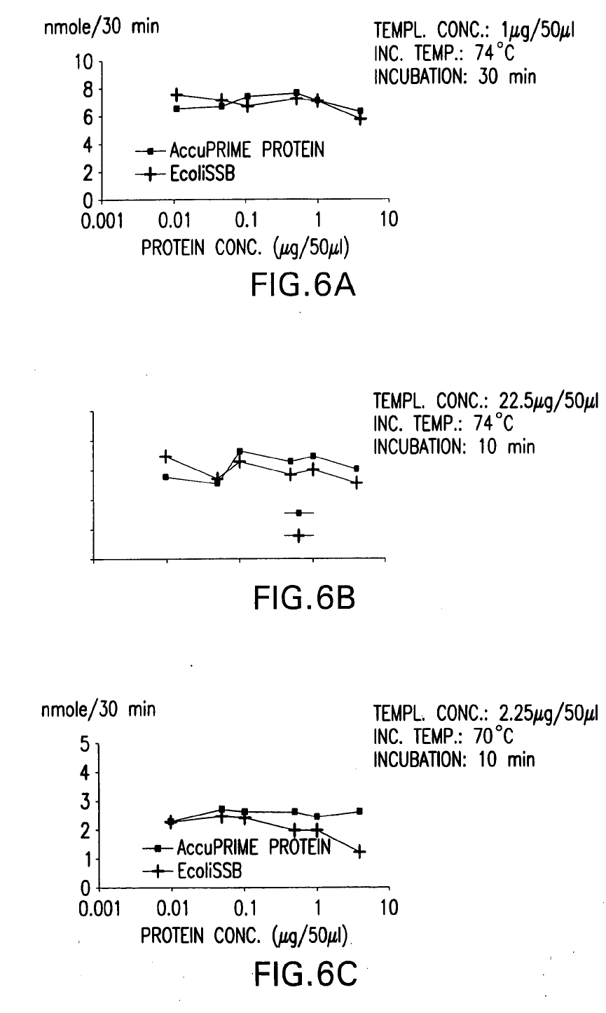

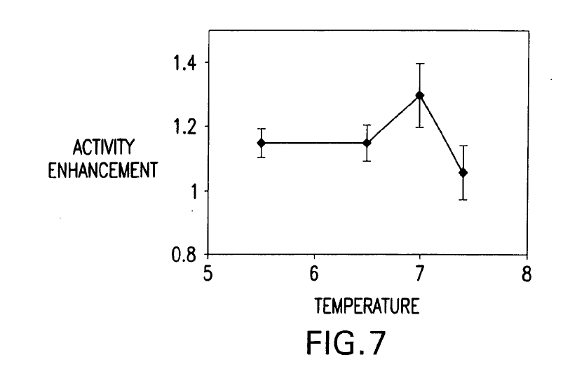

With the nicked and gapped salmon testes DNA there was a increase in the unit activity by about 10 to 40% depending on the template concentration and the incubation temperature (FIG. 6). Enhancement was more pronounce at a lower template concentration (below 0.05 μg/μl, or 2.5 μg in a 50 μl reaction), as if the enhancement would be more detectable in a less optimal (unsaturating) condition. However the temperature effect in the enhancement was less obvious at the beginning. A systematic study with the temperature variation revealed that the enhancement was at the maximum at around 70° C. and gradually decreased either at higher or lower temperatures (FIG. 7). The temperature effect suggests that there were at least two independent factors involved in enhancing the Taq DNA polymerase unit activity by AccuPrime protein. Considering that the temperature optimum for the polymerase activity itself was at 74° C., a negative factor might be taking over at 70° C. or higher.

Almost no detectable enhacement in the polymerase unit activity was observed with pre-primed circular ssDNA template regardless of the variation in the template concentration and incubation temperature. These results strongly suggest that the enhancement in Taq DNA polymerase unit activity by AccuPrime protein is through the recruitment of the polymerase to the primed sites.

Effect on elongation activity of Taq polymerase. The polymerase unit assay based on the incorporation of radioactive nucleotides into acid-insoluble fraction is suitable to measure polymerase activity in that it can provide the rate of incorporation quantitatively. However, it lacks in showing other characteristics of the enzyme, such as processivity and fidelity. Processivity of a polymerase could be assessed by analyzing the elongation product on denaturing agarose gel electrophoresis.

Elongation was done similarly with the unit assay using 5′ radio labeled primer annealed to circular ssDNA template. At predetermined time points after addition of nucleotide mix, an aliquot was retrieved and reaction was terminated by mixing EDTA to the final concentration of 0.1M. The elongation product was concentrated by ethanol precipitation and redissolved in alkaline gel loading buffer. The concentrated samples were loaded onto 1% alkaline agarose gel. After electrophoresis the gel was dried and autoradiographed.

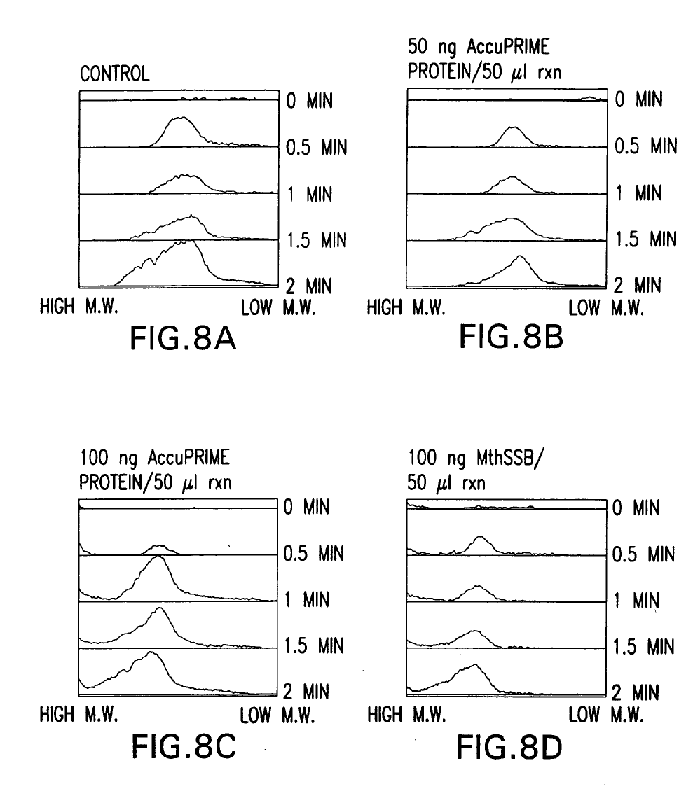

The gel showed that there was a noticeable change in the length distribution of the elongation products by adding AccuPrime protein protein to the reaction (FIG. 8). In presence of the protein the majority of the population of extended molecules was shifted to shorter molecules, compared to those in the absence of the protein. This result suggests that AccuPrime protein made Taq DNA more distributive. It is not still clear the if the protein enhanced initiation step of the polymerase or prevented the enzyme from carrying on polymerization once initiated by this experiment alone. When considering the result of the unit assay result above, it could be deduced that AccuPrime protein helps Taq DNA polymerae to be loaded on to the primed sites.

Stability of AccuPrime Protein in AccuPrime Formulation

Accelerated stability assay. All four formula of AccuPrime Taq PCR reaction mixes (10× AccuPrime Taq PCR Reaction Mix I and II, and 2× AccuPrime Taq PCR SuperMix I and II) were subject to accelerated stability test using two different incubation temperatures, 37 and 45° C., for duration, 7 and 4 days, respectively, that are equivalent to storage at −20° C. for a year. After a period of incubation, the reaction mixes were used in PCR reactions similar to that of functional QC.

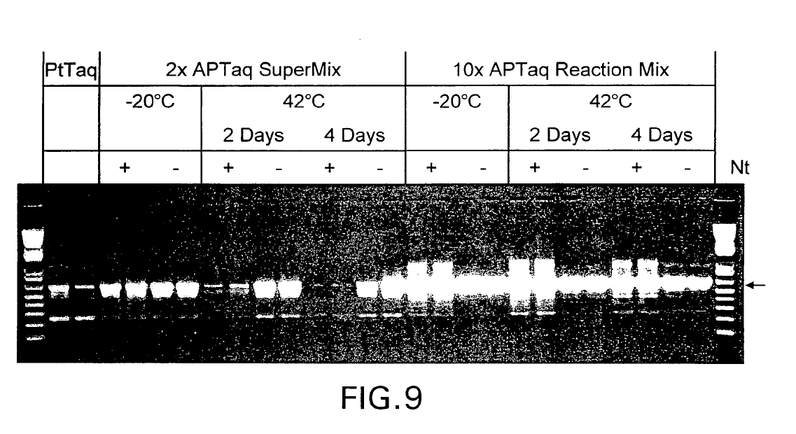

The PCR product was assayed on 1% agarose gel electrophoresis with Ethidium bromide staining. The visual inspection of the band intensity for specific and non-specific product showed that the reaction mixes after incubation functioned just as well as those in control (FIG. 9). The control reaction mixes were stored frozen at −20° C. and never subject to a high-temperature incubation other than thawing right before use, or freshly made just before uses. This result indicates that the AccuPrime protein was stable in storage at −20° C. up to a year in the Reaction Mixes at both high and low concentrations, and in combination with AccuPrime Taq DNA polymerase.

Real-time stability assay. Though the accelerated stability assay provided us with the necessary information about the stability of the reaction mixes, a real-time stability test is preferred simply due to the fact that it is real and not simulated. Reaction mixes in small aliquots (each aliquot contains the reaction mix equivalent to 10 50 μl PCR reactions) stored at three different temperatures, −20° C., 4° C. and room temperature, and at pre-determined time points a few aliquots were retrieved from incubation and stored at −20° C. until the PCR functional assay. The PCR functional assay was performed in the identical manner as in the accelerated stability assay.

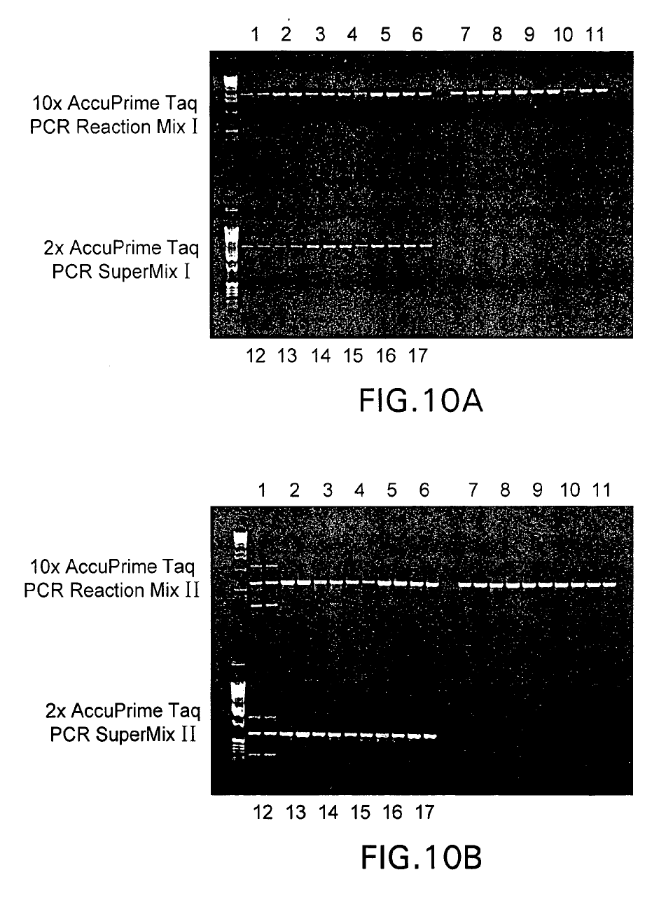

As of this writing, 6-month incubation was completed and all reaction mixes were found to be functional after 6-month incubation at room temperature (FIG. 10). However, it was also found that a series of freeze-and-thaw cycles (more than 5 times) were more detrimental in its functionality, especially with the SuperMix formula, than keeping them at room temperature for the period. This result may indicate that the aliquoting the reaction mix should be strongly encouraged, and that an aliquot should be stored at 4° C., if it is frequently used.

Post-PCR Assay for AccuPrime Taq DNA Polymerase Amplification

TOPO TA cloning. The transformation efficiency right after the TOPO TA cloning, assuming all other parameter remained same, was a little lower for the amplification product from AccuPrime Taq DNA polymerase (70% for the multi-cloning site amplicon and 37% for the GC rich amplicon) than the Platinum control. The lower transformation efficiency might have resulted from the high affinity to ssDNA by the AccuPrime protein that carried over to the cloning and transformation, and might have caused a problem for one of the α testers.

However, out of 6 colonies randomly selected for each transformation, 5 transformants from each amplicon amplified by AccuPrime Taq showed the right insert, compared to only 4 for GC rich amplicon and 6 for the multi-cloning site amplicon by the Platinum Taq PCR. This result indicated less discrimination between amplicons by AccuPrime Taq DNA polymerase.

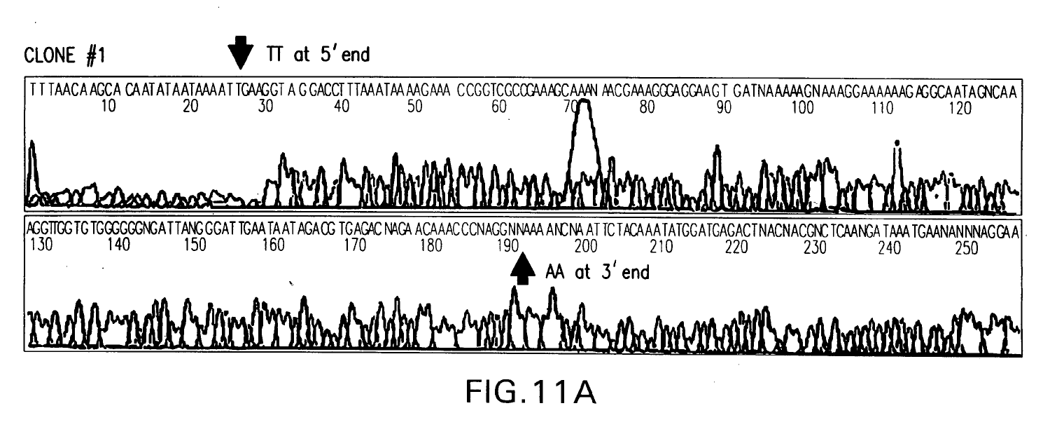

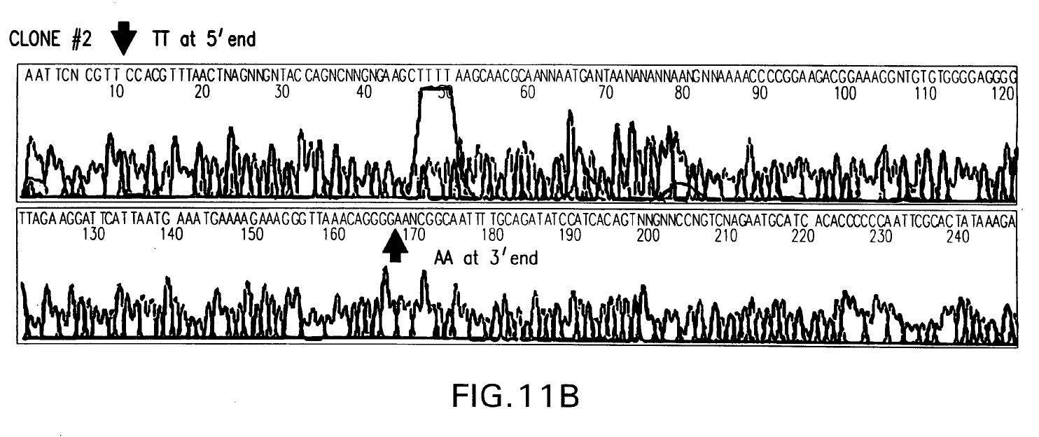

More conclusive evidence that the amplification products were compatible with TOPO TA cloning was provided by the sequencing result (FIG. 11). The sequencing clearly demonstrated that the insert into the pCR2.1-TOPO vector was flanked by TT at the 5′ end and AA at the 3′ end. This result indicates that AccuPrime Taq DNA polymerase rightly produces 3′ A overhang necessary for TOPO TA cloning.

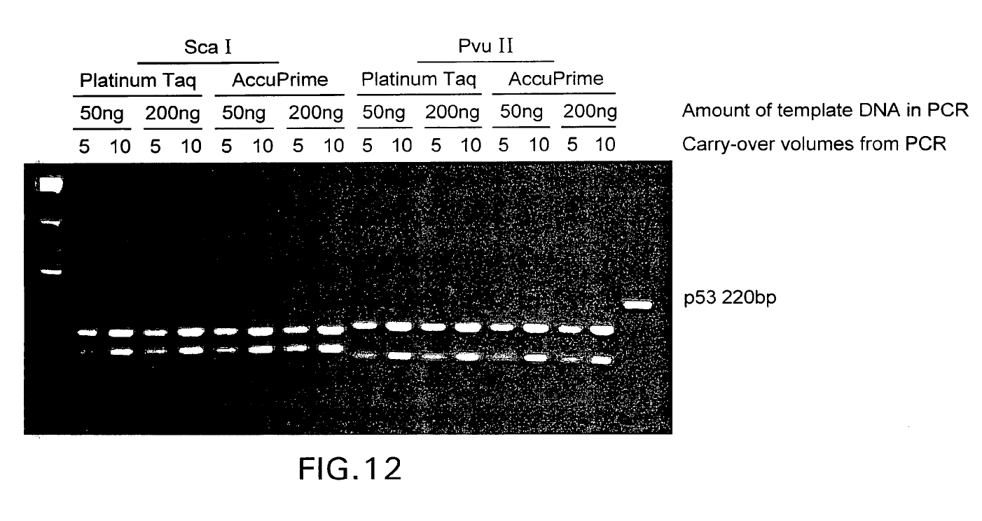

Restricition Endonuclease Digestion. The compatibility of amplification product of the AccuPrime Taq DNA polymerase to RFLP assay was analyzed by using the amplification product from PCR reaction directly in the digestion reaction with two different restriction enzymes. For up to 50% of reaction volume carried over from PCR reaction, there was no detectible hindrance observed for restriction digestion (FIG. 12). This result indicates that whatever the component that might have carried over to the digestion reaction did not interfere with the enzyme digestion of the amplification product.

PCR Application Development for AccuPrime Taq DNA Polymerase

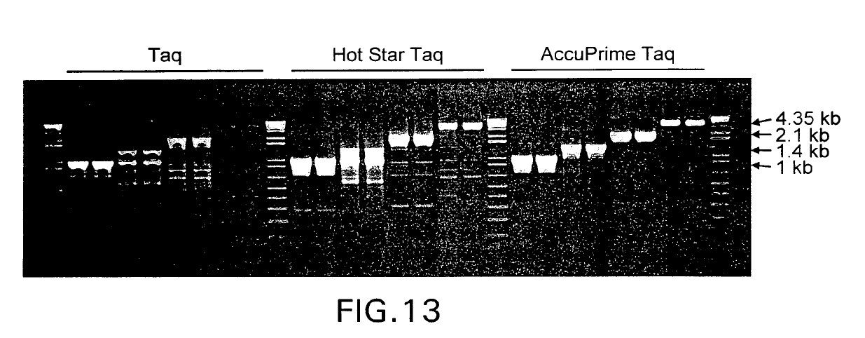

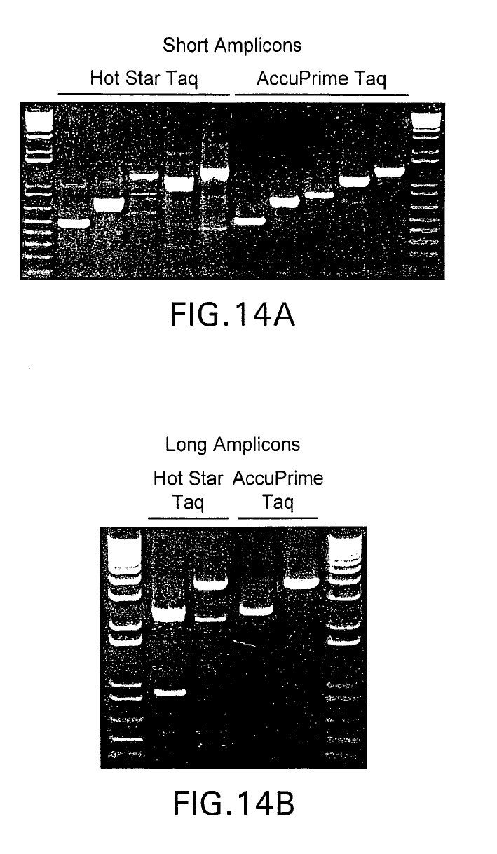

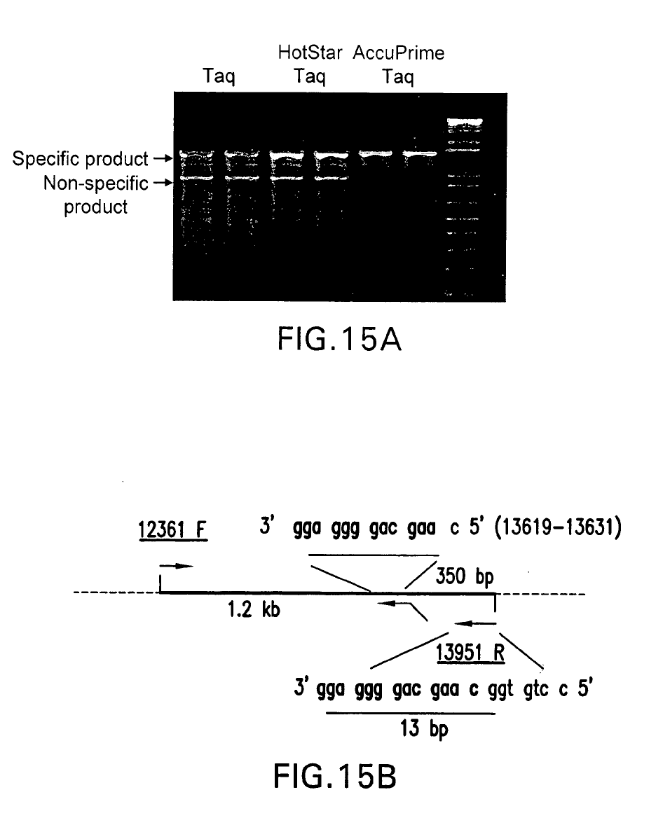

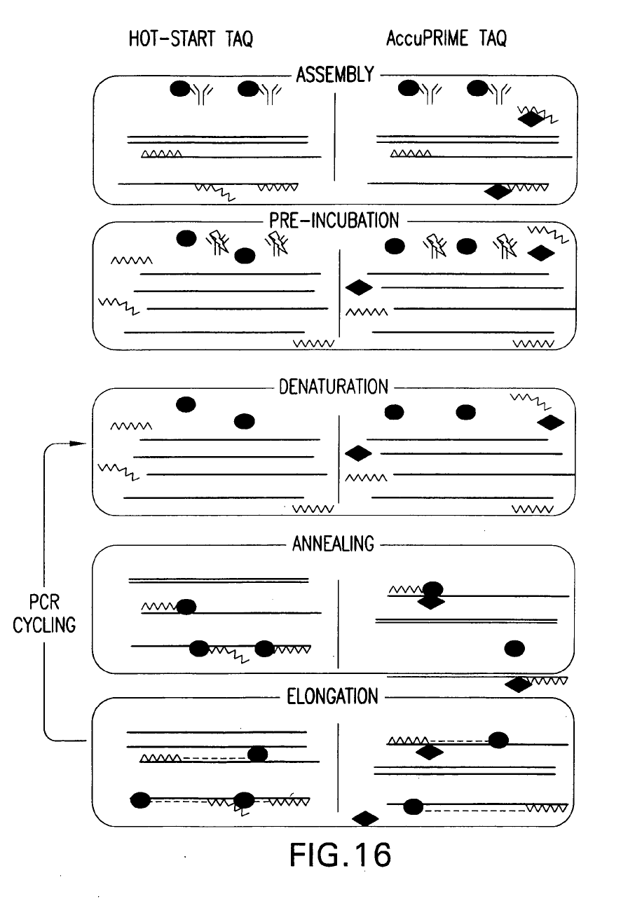

Specificity enhancement of AccuPrime Taq DNA polymerase. In performance comparison using 6 primer sets with amplicons ranging from 264 to 4,350 bp (Pr 1.3, 264 bp; Rhod, 646 bp; β-globin, 731 bp; Hpfh, 1,350 bp; p53, 2,108 bp; p53, 4,350 bp), AccuPrime Taq DNA polymerase out performed Taq DNA polymerase and all other hot start polymerases (AmpliTaq Gold, Perkin Elmer; Jump Start, Sigma; Fast Start, Roche; Hot Star Taq, Qiagen; Sure Start, Stratagene). AccuPrime Taq shows the highest specificity and consistent yields regardless of the amplicon sizes (FIGS. 13 & 14). The yields from the AccuPrime Taq DNA polymerase are among the highest. In more detailed surveys, AccuPrime Taq DNA polymerase required less optimization in terms of primer annealing or amplicon size to obtain consistent high specificity than the gold standards of current market, AmpliTaq Gold (Perkin Elmer) (FIG. 15) or HotStar Taq (Qiagen) (FIG. 16).

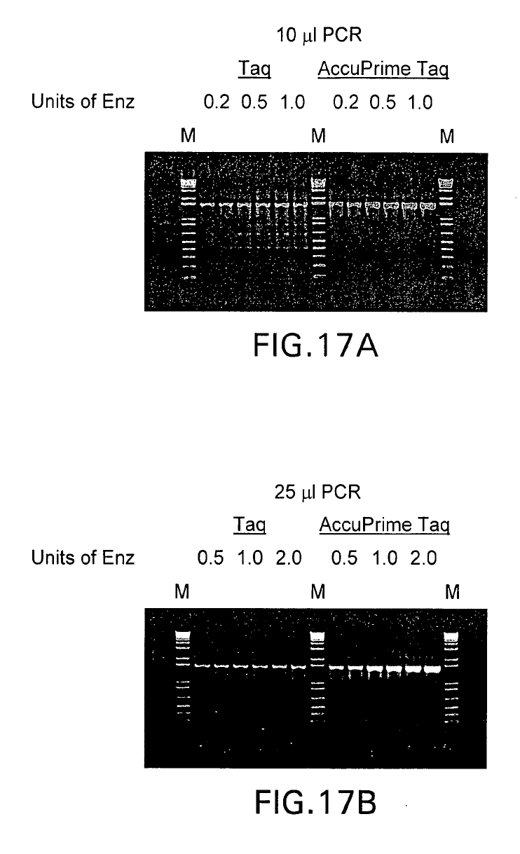

To establish its ability to suppress non-specific priming, a primer was designed so that it would fully anneal to a site and partially (13 bp at the 3′ end) anneal to another 350 bp downstream from the full-annealing site. It was achieved by taking advantage of an amplification target that had 13-bp homology to the downstream site. Unlike HotStar Taq or Taq alone that could not descriminate against the partial annealing site, AccuPrime Taq was able to suppress false priming to produce only the specific product (FIG. 17). This result re-emphasize the advantage the AccuPrime Taq had over other hot-start enzyme where AccuPrime functions throughout PCR cycling to prevent non-specific priming.

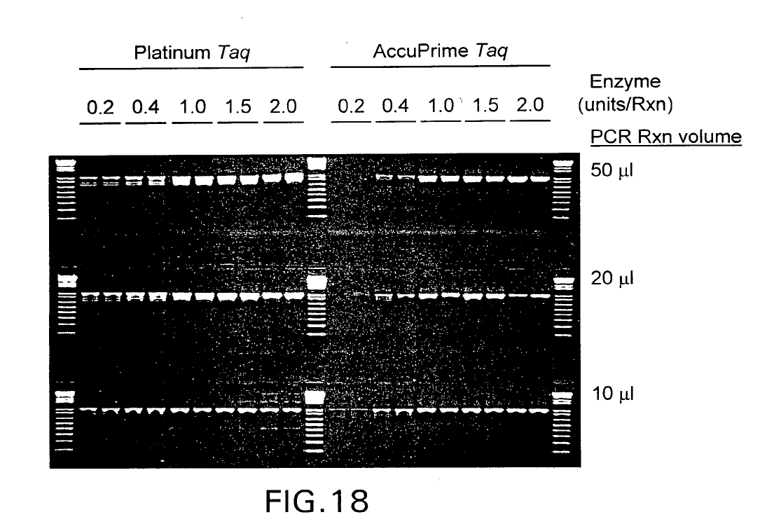

In PCR AccuPrime protein enhances Taq DNA polymerase activity in its sensitivity, specificity and fidelity (Table 6). Such enhancement makes AccuPrime Taq DNA polymerase system suitable to many areas of PCR application, such as, high throughput PCR, multiplex PCR and PCR miniaturization (Table 7). We propose here a mechanism that explains its role in PCR. The protein seems to stabilize the specific primer:template interaction, to compete off non-specific primer annealing, and to recruit Taq DNA polymerase to the primed sites (FIG. 18). As a result, it utilizes available resources to the specific primer elongation reaction in PCR where the primers and the polymerases repeat annealing and elongation at each reaction cycle.

TABLE 6

| | | No difference | Total |
|---|---|---|---|
| Great | Slight | (including already | primer |
| Improvement | Improvement | good primers) | sets |
| 120 (40%) | 105 (35%) | 75 (25%) | 300 |

TABLE 7

| | Amplitaq | | |
|---|---|---|---|
| Features | Gold | AccuPrime Taq | PlatinumTaq |
| Automatic Hot | + | + | + |
| Start | | | |
| Accurate | − | + | − |
| Priming | | | |
| Length of PCR | 0.1-2 Kb | 0.1-4 Kb | 0.1-4 Kb |
| Product | | | |
| Reactivation of | 10 min, at | 0.5-2 min at 94° C. | 0.5-2 min at |
| Taq Activity | 95° C. | (standard PCR) | 94° C. |
| | (pre- | | (standard PCR) |
| | incubation | | |
| | is required) | | |
| Template | ? | 5 ng-200 ng | 5 ng-200 ng |
| Requirements | | | |
| (Genomic DNA) | | | |
| Degree of | Moderate | Low | Low |
| Optimization | | | |
| Fidelity | Standard | 2-fold better than | Standard Taq |
| | Taq | Taq alone | |
| Difficult | − | ++ | + |
| Templates | | | |
| (GC-Rich, n.t | | | |
| repeats) | | | |
| Templates | − | + | − |
| w/Secondary | | | |
| Structure | | | |
| Specificity | + | +++ | + |
| Sensitivity | + | +++ | ++ |
| Yield | + | +++ | +++ |

Miniaturization. The concept of miniaturization was first conceived as a cost-effective way of doing PCR reactions. The focus was placed on three different volumes of reactions, 10 μl, 20-25 μl, and, the standard 50 μl reaction as a control. Titrations and optimization experiments were conducted for each component of a typical PCR reaction, such as dNTP, primer, AccuPrime Taq DNA polymerase, and specifically for this product, AccuPrime protein (single stranded DNA binding protein, or AccuPrime protein).

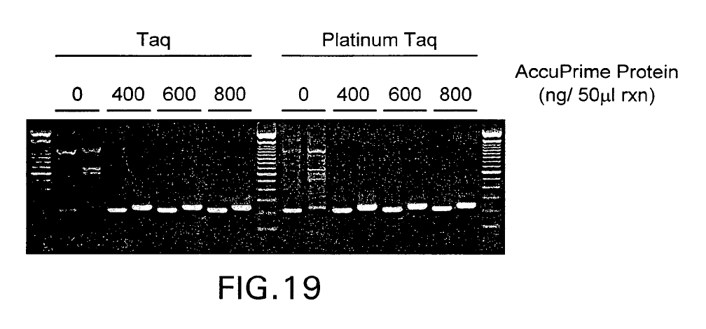

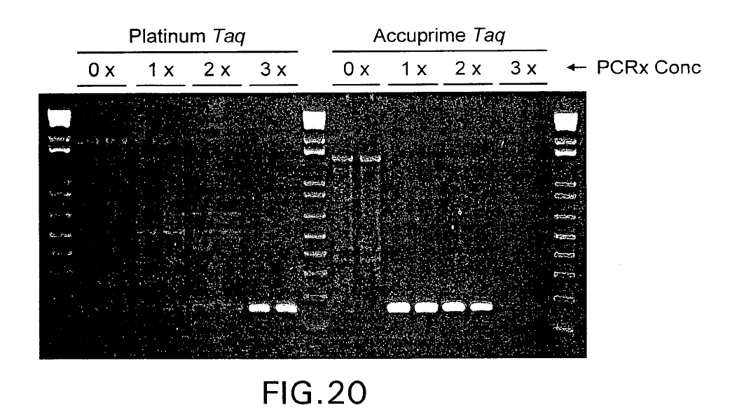

From our experiments we found that the concentrations of dNTP's, buffers, and primer could be proportionally reduced and still maintained the high performance seen with standard 50 μl reactions (FIGS. 19 & 20). The combined results are summarized in Table 8. Most significantly, we also found that the required amounts of AccuPrime Taq DNA polymerase to be 2 to 3 times less than either Taq or Platinum Taq in producing the same quality PCR products.

TABLE 8

| | | Reaction Volume | | | | | |
|---|---|---|---|---|---|---|---|
| | | 10 ul | | | 25 ul | | |
| Primer Enzyme | | 0.2 u | 0.5 u | 1.0 u | 0.5 u | 1.0 u | 2.0 u |
| p53 | Taq¹ | + | ++ | +++ | ++ | +++ | +++ |
| (504 bp) | Pt² | + | ++ | +++ | ++ | +++ | +++ |
| | AP³ | +++ | +++ | +++ | +++ | +++ | +++ |
| c-myc | Taq | ++ | ++ | +++ | + | ++ | +++ |
| (1069 bp) | Pt | ++ | ++ | +++ | + | ++ | +++ |
| | AP | +++ | +++ | +++ | +++ | +++ | +++ |
| p53 | Taq | ++ | ++ | +++ | ++ | ++ | +++ |
| (1587 bp) | Pt | ++ | +++ | +++ | ++ | +++ | +++ |
| | AP | +++ | +++ | +++ | +++ | +++ | +++ |
| p53 | Taq | ++ | ++ | +++ | +++ | +++ | +++ |
| (1996 bp) | Pt | ++ | ++ | +++ | +++ | +++ | +++ |
| | AP | ++ | +++ | +++ | +++ | +++ | +++ |
| Rhod | Taq | + | ++ | +++ | ++ | +++ | +++ |
| (3047 bp) | Pt | ++ | +++ | +++ | +++ | +++ | +++ |
| | AP | +++ | +++ | +++ | +++ | +++ | +++ |
| p53 | Taq | − | − | − | − | + | +++ |
| (4356 bp) | Pt | + | + | ++ | ++ | ++ | +++ |
| | AP | + | +++ | +++ | ++ | +++ | +++ |

¹Taq: Taq DNA polymerase alone
²Pt: Platinum Taq DNA polymerase
³AP: AccuPrime Taq DNA polymerase

Difficult templates. Templates with high GC content (>70%) are notoriously difficult to amplify due to their high melting temperature. It was our intension to improve specificity and yield of these templates using the AccuPrime Taq PCR reaction mix.

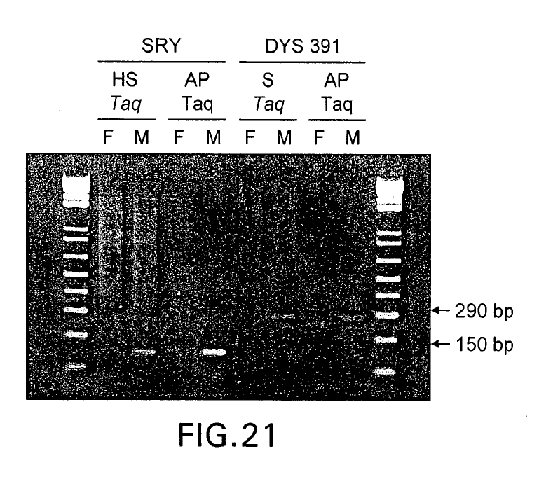

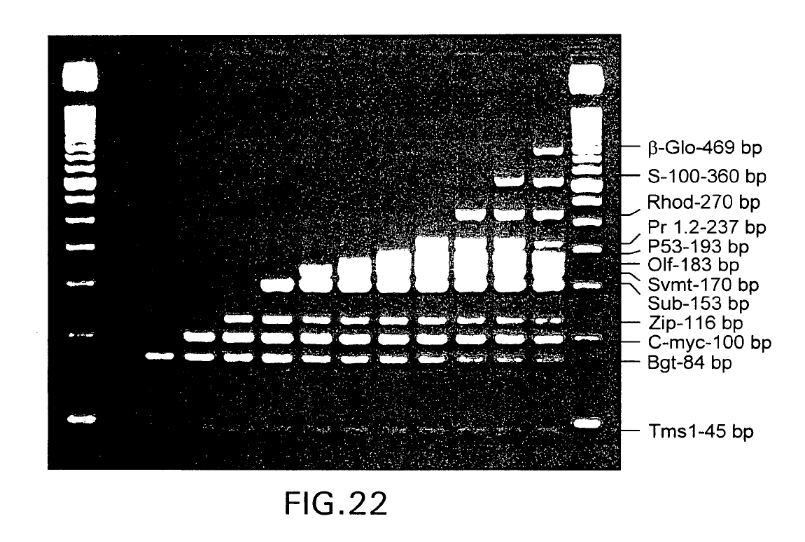

Titration of AccuPrime protein showed that 1× concentration of SSB was good enough to obtain specific product where Platinum Taq failed (FIG. 21). However, an attempt to improve specificity by optimizing the annealing temperature did not prove successful. By substituting Hi-Fi Buffer and MgSO₄ for the standard PCR buffer and MgCl₂, 50% of the samples tested showed an improvement in specificity. Independently, the addition of PCRx enhancer solution to a standard PCR reaction mixture showed improvement in the specificity when used in combination with the AccuPrime Taq. In addition, the yield was found to be 3 times higher than that of Platinum Taq (FIG. 22). In combination, the Hi-Fi buffer, MgSO₄, and the PCRx enhancer solution together resulted in another 3 fold improvement of product performance over Platinum Taq.

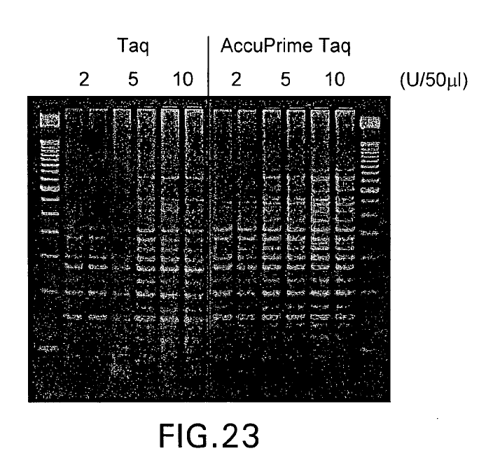

With superior specificity and its ability to amplify difficult templates, AccuPrime Taq DNA would be ideal in applications, such as PCR genotyping. Feasibility for usage of AccuPrime Taq in genotyping was tested using two independent genomic targets. Both the genes, SRY and DYS-391, reside in Y chromosome so that the only male genomic DNA would have the specific targets. In both cases AccuPrime Taq (AP Taq) showed specific amplication product while suppressing background. The control HotStar Taq (HS Taq) showed many non-specific products in the background especially in SRY gene (FIG. 23).

Multiplex PCR. The use of multiplex PCR serves a desirable, practical purpose in that it saves time, labor, and cost for the end user. However, in all practical purposes, optimization of multiplex PCR can be tedious and time consuming, partly due to the high probability of cross-interaction between different primer pairs, and to the difficulties in optimizing each set primers to perform equally with others together in a reaction. Encouraged by the specificity enhancing properties of AccuPrime Taq, feasibility of a practical multiplex PCR was tested using AccuPrime Taq PCR reaction mix.

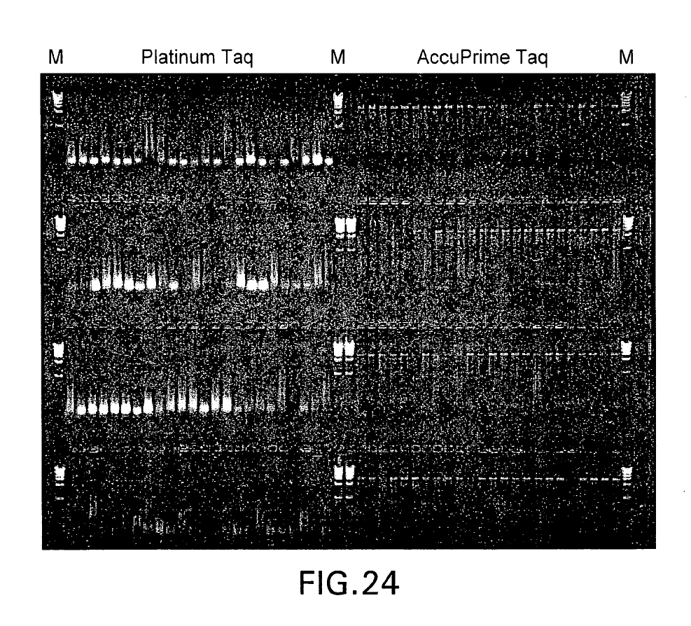

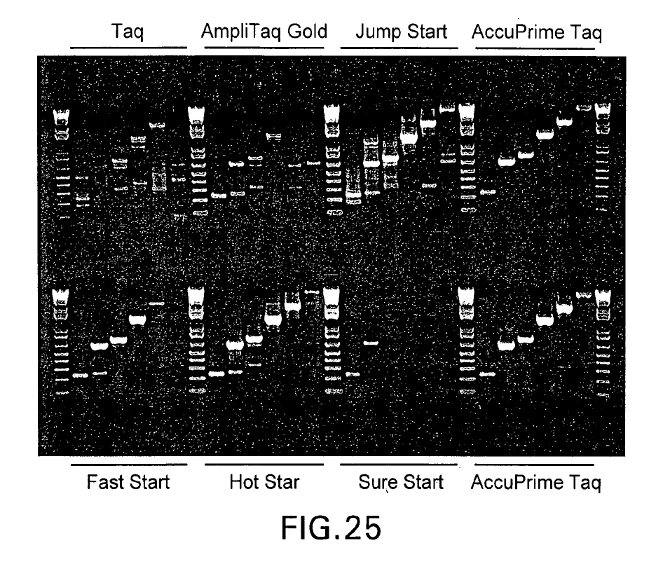

Upon completion of these experiments it was found that the optimal PCR conditions for multiplex did not require much optimization, other than using the standard conditions as in a standard PCR reaction (FIG. 24): A typical multiplex PCR reaction would contain 0.2 mM dNTP, 1.5 mM MgCl₂, and 400 ng of AccuPrime protein. We also found that 2 units of AccuPrime Taq DNA polymerase was sufficient for multiplex PCR between 2-10 primers sets. Beyond that, up to as many as 20 sets it required 5 units of enzyme per reaction to achieve optimal results (FIG. 25).

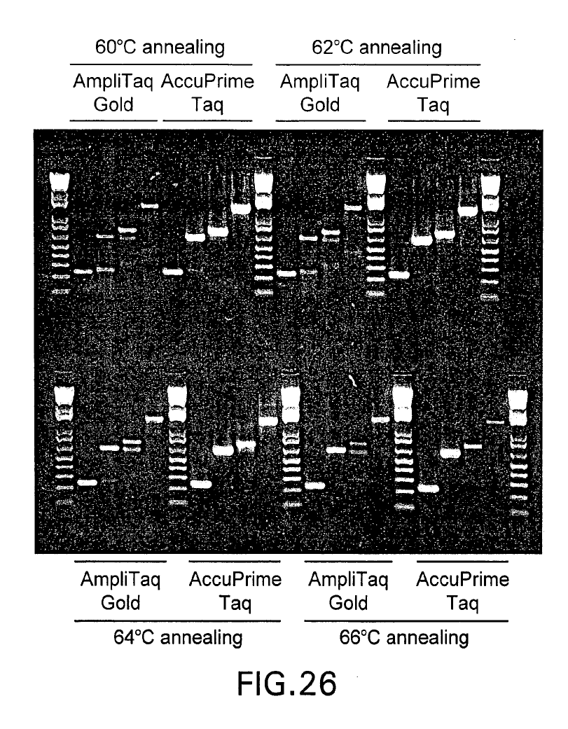

High throughput PCR. AccuPrime Taq DNA polymerase improved the robustness of high throughput screening reducing total cycling number and increasing specificity when compared with Platinum Taq DNA polymerase (FIG. 26).

Fidelity Enhancement

Enhanced fidelity of the Accuprime Taq DNA Polymerase was confirmed using the rpsL fidelity assay (Lackovich et al., 2001; Fujii et al., 1999). The fidelity of AccuPrime Taq DNA polymerase was determined using the rpsL fidelity assay. Taq DNA polymerase from the Invitrogen Corporation was used as a control. The error rate of AccuPrime Taq DNA polymerase was determined to be 1.72×10⁻⁵ (Table 9). Over the course of three independent fidelity runs, AccuPrime Taq DNA polymerase showed nearly a two fold improvement in fidelity over Taq DNA polymerase each time. Mutant colonies were PCR amplified with rpsL primers and gel analyzed to verify the existence of the mutant gene.

TABLE 9

| | | | Transformants | TD | | | |
|---|---|---|---|---|---|---|---|
| | | Total | with mutant | (doubling | mf | | Relative |
| Enzyme | Exp. | Transformant | rpsL | time) | (%) | Er (×10⁻⁶) | Fidelity |
| Taq | 1 | 999 (3,996) | 235 (1.2 mL) | 12.3 | 5.88 | 36.8 | 1X |
| | 2 | 906 (9,060) | 420 (600 ul) | 12.3 | 4.64 | 29.0 | |
| | 3 | 731 (14,260) | 616 (600 ul) | 11.6 | 4.32 | 28.6 | |
| | Avg | | | 12.1 + 0.3 | 4.95 + 0.62 | 31.5 + 3.57 | |
| AP | 1 | 732 (7,320) | 234 (1.2 mL) | 11.6 | 3.20 | 21.2 | 1.8X |
| | 2 | 942 (28,260) | 633 (600 ul) | 11.6 | 2.24 | 14.9 | |
| | 3 | 798 (15,960) | 330 (600 ul) | 10.2 | 2.07 | 15.6 | |
| | Avg | | | 11.1 + 0.6 | 2.50 + 0.47 | 17.2 + 2.63 | |

### CONCLUSION

We report development of a next generation PCR enzyme systems, AccuPrime Taq DNA polymerase system and AccuPrime Taq PCR SuperMix. These systems incorporate a thermostable AccuPrime protein to the Platinum technology that enhances Taq DNA polymerase activity in PCR. AccuPrime protein is the thermostable SSB from Methanococcus jannaschii and unlike other SSB consists of a single polypeptide chain. The cloned gene was obtained from Dr. Stephen C. Kowalczykowski from UC, Davis and the protein was purified to 95% purity using a modified purification protocol. In PCR it enhances Taq DNA polymerase activity in its sensitivity, specificity and fidelity. Such enhancement makes AccuPrime Taq DNA polymerase system suitable to many areas of PCR application, such as, high throughput PCR, multiplex PCR and PCR miniaturization. It is also shown that the AccuPrime protein enhancement of the polymerase activity is specific to Taq DNA polymerase. Since AccuPrime protein and Taq DNA polymerase come from two rather independent organisms, the interaction could be structural-homology driven.

In summary, the AccuPrime protein enhances the activity of Taq DNA polymerase and in PCR improves the specificity drastically. Unlike other hot-start DNA polymerases, it improves PCR performance by promoting specific primer-template hybridization before as well as during every cycle of PCR. All commercially available hot-start Taq DNA polymerase, either by chemically modification or anti-Taq antibody addition, designed to block DNA polymerase activity before PCR cycle but not during PCR cycles. In a PCR study using more than 300 primer sets, AccuPrime Taq™ DNA polymerase showed improvement in yield, sensitivity and/or specificity over other hot-start PCR enzymes in 75% of the cases. While its sensitivity and specificity makes the new Amplification enzyme ideal to variety of PCR/RT-PCR applications, its robustness reduces the need for optimization to the minimum for any particular PCR application. In high throughput or multiplex format, such as, genotyping, colony PCR and PCR miniaturization, the new PCR enzyme out-performs all premier gold standard enzymes in the current PCR market. It is also demonstrated that AccuPrime protein improves the fidelity of Taq DNA polymerase by 2 fold.

System configurations are provided in Table 10.

TABLE 10

| Name | Components |
|---|---|
| AccuPrime Taq | 2X PCR Buffer [40 mM Tris-HCl (pH 8.4), 100 mM |
| PCR Supermix I | KCl] |
| (for small | 3 mM MgCl₂ |
| genomic DNA | 400 uM dGTP |
| targets less than | 400 uM dATP |
| 200 bp, plamid | 400 uM dTTP |
| DNA or cDNA | 400 uM dCTP |
| templates) | 0.1% Tween 20 |
| | 0.1% Nonidet P-40 |
| | 80 units/ml rTaq DNA polymerase (1 unit rTaq DNA |
| | polymerase per 25 ul reaction) |
| | 1.6 ug/ml of each of two anti-Taq DNA polymerase |
| | antibodies (1:5) |
| | 2 ug/ml Mja RPA SSB (25 ng Mja SSB per 25 ul |
| | reaction) |
| AccuPrime Taq | 2X PCR Buffer [40 mM Tris-HCl (pH 8.4), 100 mM |
| PCR Supermix II | KCl] |
| (for genomic | 3 mM MgCl₂ |
| DNA templates | 400 uM dGTP |
| in the range of | 400 uM dATP |
| 200 bp to 4 kb in | 400 uM dTTP |
| size) | 400 uM dCTP |
| | 0.1% Tween 20 |
| | 0.1% Nonidet P-40 |
| | 80 units/ml rTaq DNA polymerase (1 unit rTaq DNA |
| | polymerase per 25 ul reaction) |
| | 1.6 ug/ml of each of two anti-Taq DNA polymeras |
| | antibodies (1:5) |
| | 16 ug/ml Mja RPA SSB (200 ng Mja SSB per 25 ul |
| | reaction) |
| AccuPrime Taq | 2 units/ul AccuPrime Taq (2 units/ul rTaq DNA |
| DNA polymerase | polymerase, 40 ng/ul each of two anti-Taq DNA |
| system | polymeras antibodies; 1:5 Molar ratio) |
| | 50 mM MgCl₂ |
| 10x Reaction | 10X AccuPrime Taq Reaction mix with AccuPrime ™ |
| Mix I (for small | protein {10X PCR buffer [200 mM Tris-HCl (pH 8.4), |
| genomic DNA | 500 mM KCl], 15 mM MgCl₂, 2 mM dGTP, 2 mM |
| targets less than | dATP, 2 mM dTTP, 2 mM dCTP, 10 ug/ml of Mja |
| 200 bp, plamid | SSB, 10% Glycerol} |
| DNA or cDNA | |
| templates) | |
| 10x Reaction Mix | 10X AccuPrime Reaction mix with AccuPrime ™ |
| II | protein {10X PCR buffer [200 mM Tris-HCl (pH 8.4), |
| (for genomic | 500 mM KCl], 15 mM MgCl₂, 2 mM dGTP, 2 mM |
| DNA templates | dATP, 2 mM dTTP, 2 mM dCTP, 80 ug/ml of Mja |
| in the range of | SSB, 10% Glycerol} |
| 200 bp to 4 kb in | |
| size) | |

### REFERENCES

Chedin, F, Seitz, E. M. and Kowalczykowski, S. C. (1998). “Novel homologs of replication protein A in archaea: implications for the evolution of ssDNA-binding proteins.” Trends Biochem. Sci. 23, 273-277
Chou, Q. (1992). “Minimizing deletion mutagenesis artifact during Taq DNA polymerase PCR by E. coli SSB.” Nucl. Acids Res. 20, 4371
Dabrowski, S. and Kur, J. (1999). “Cloning, overexpression, and purification of the recombinant His-tagged SSB protein of Escherichia coli and use in polymerase chain reaction amplification.” Protein Expr. Purif. 16 (1), 96-102
Fujii, S., Akiyama, M., Aoki, K., Sugaya, Y., Higuchi, K., Hiraoka, M., Miki, Y., Saitoh, N., Yoshiyama, K., Ihara, K., Seki, M., Ohtsubo, E. and Maki, H. (1999). “DNA replication errors produced by the replicative apparatus of Escherichia coli.” J. Mol. Biol. 289, 835-850
Glover, B. P. and McHenry, C. S. (1998). “The χψ subunits of DNA polymerase III holoenzyrne bind to single-stranded DNA-binding protein (SSB) and facilitate replication of an SSB-coated template.” J. Biol. Chem. 273, 23476-23484
Kelly, T. J., Simancek, P. and Brush, G. S. (1998). “Identification and characterization of a single-stranded DNA-binding protein from the archaeon Methanococcus jannaschii.” Proc. Natl. Acad. Sci. USA 95, 14634-14639
Kim, Y. T., Tabor, S., Churchich, J. E. and Richardson, C. C. (1992). “Interactions of gene 2.5 protein and DNA polymerase of bacteriophage T7.” J. Biol. Chem. 267, 15032-15040
Kim, Y. T. and Richardson, C. C. (1994). “Acidic carboxyl-terminal domain of gene 2.5 protein of bacteriophage T7 is essential for protein-protein interactions.” J. Biol. Chem. 269, 5270-5278
Lackovich, J. K., Lee, J. E., Chang, P. and Rashtchian, A. (2001). “Measuring fidelity of Platinum Pfx DNA polymerase.” Focus 23, 6-7
Lee, J., Chastain, P. D., Jr., Kusakabe, T., Griffith, J. D. and Richardson, C. C. (1998). “Coordinated leading and lagging strand DNA synthesis on a minicircular template.” Mol. Cell 1, 1001-1010
Rapley, R. (1994). “Enhancing PCR amplification and sequencing using DNA-binding proteins.” Mol. Biotechnol. 2, 295-298

### Example 5

### Development and Characterization of the Accuprimepfx DNA Polymerase System

Introduction

A highly thermostable AccuPrime protein has been successfully integrated with KOD DNA polymerase (Toyobo (aka PfX™ DNA polymerase)) and with antibodies specific to KOD DNA polymerase to generate a next generation high fidelity PCR enzyme—AccuPrime Pfx™ DNA polymerase. The AccuPrime technology has been already integrated to and proven successful in AccuPrime Taq DNA polymerase. We have optimized the AccuPrime Pfx™ DNA polymerase for PCR applications that requires high fidelity, high sensitivity and robustness.

Most of the high fidelity enzymes currently available in the market require extensive optimization, with no guarantee for specific products. By introducing AccuPrime proteins to Platinum Pfx DNA polymerase we are able to make the enzyme robust and reproducible in PCR reactions. Some of the commercially available high-fidelity PCR enzymes employ one of the hot-start technologies, either by chemical modification or by addition of antibodies to block the polymerase activity during the assembly of the reaction. However, most of the existing hot-start technologies functions before but not during PCR cycles. Unlike other hot-start DNA polymerases, AccuPrime technology improves PCR performance by promoting specific primer-template hybridization before as well as during every cycle of PCR. The AccuPrime technology for Pfx DNA polymerase is updated to include a second, complementary AccuPrime protein, AccuPrime Protein II, that enhances robustness of the enzyme. The blending of AccuPrime proteins (two or more) is unique to AccuPrime Pfx DNA polymerase in making of a robust high-fidelity PCR enzyme.

In a PCR study using more than 90 primer sets targetting genomic DNA, plasmid, bacteriophage γ DNA or cDNA, AccuPrime Pfx™ DNA polymerase showed improvement in yield, sensitivity and/or specificity over hot-start enzymes in about 50% of the cases overall. With its enhanced sensitivity, specificity and reproducibility, AccuPrime Pfx DNA polymerase is ideal to variety of PCR/RT-PCR applications, while its robustness reduces the need for optimization to the minimum. AccuPrime Pfx DNA polymerase complement very well with AccuPrime Taq DNA polymerase in generating a premier, next-generation PCR enzyme family.

It was believed that single stranded DNA binding protein (SSB) would help in PCR in terms of the specificity, yield and sensitivity, on the basis of the function of the protein in DNA replication system. In DNA replication, helicases unwind double stranded (ds) DNA into two complementary single strands (ss), necessary for the functions of primases and DNA polymerases. SSB protects ssDNA template simply by coating the molecules and, while doing so, prevents the ssDNA from base pairing with the complementary strand. There also exists a set of evidence that SSB may directly interact with DNA polymerases in a species-specific manner (Kim et al., 1992; Kim and Richardson, 1994; Glover and McHenry, 1998; Lee et al., 1998).

The most obvious reason for SSB to enhance PCR reaction would be its ability to remove secondary structures (hairpins and such) from the template, and to maintain the DNA template single-stranded. In fact, it has been reported that SSB from E. coli and other mesophilic organisms improved PCR efficiency (Chou, 1992; Rapley, 1994; Dabrowski and Kur, 1999). However, due to the thermo-labile nature of the mesophilic SSB, the enhancement by the proteins were too limited to be practical in PCR application where the cycling incubation temperatures exceed the upper limit of their thermostability.

The existence of a thermostable SSB from an archaeon was first reported by Dr. Stephen C. Kowalczykowski from UC, Davis (Chedin et al., 1998) and its gene was subsequently cloned by Thomas J. Kelly's group in the Johns Hopkins University (Kelly et al., 1998). Subsequently, a few more archaeal SSB have been cloned and purified by other groups. We have used two SSB proteins from UC, Davis (the protein will be referred to as “AccuPrime protein I” and “AccuPrime protein II” hereon) and studied its effect on DNA polymerase activity and fidelity, resulting in the development of AccuPrime Taq DNA polymerase and AccuPrime Pfx DNA polymerase described herein. The improvement on AccuPrime technology for AccuPrime Pfx include adding a second, complementary SSB of different origin to the existing SSB. Unlike the popular belief that SSB proteins of different origins share an extensive homology in structure and function, AccuPrime Pfx DNA polymerase shows two different SSB function differently but in a complementary manner in enhancing the performance of the Pfx DNA polymerase.

This manuscript reports our endeavor in creating a next generation, high-fidelity PCR enzyme embracing the new AccuPrime technology.

Materials and Methods

Small Scale Purification of AccuPrime Proteins I and II

AccuPrime Protein I. The purification of AccuPrime protein I has been reported above for AccuPrime Taq DNA polymerase system. Briefly, the plasmid containing the AccuPrime protein gene was transformed into BL21(DE3) cells freshly for each protein purification. The culture media (500 ml Terrific Broth, supplemented with 50 μg/ml Kanamycin) was inoculated from a starter culture, incubated at 37° C. to the 1 OD₆₀₀ (4 to 6 hours), and induced with I mM IPTG. Pelleted cells were resuspended in Lysis buffer (2 ml per g of cell pellet; 0.5M NaCl, 50 mM potassium phosphate, pH8.0, 0.25 mM PMSF, 10 mM imidazole), containing the protease inhibitor cocktail (Sigma, P 8849; 1 ml of the cocktail per 20 g of cell pellet), lysed by sonication and clarified by centrifugation. Ni-NTA agarose (Qiagen) column chromatography followed by ssDNA agarose column and MonoQ column chromatography yielded about 25 mg of 90% pure protein.

AccuPrime Protein II. Host cells, BL21(DE3), containing pET21+rSsoSSB were incubated at 30° C. in LB media supplemented with 100 μg/ml ampicillin to an OD₆₀₀ of 1.0, and induced by IPTG to a final concentration of 1 mM for two hours. Cells were harvested by centrifugation, resuspended in 2 ml of a lysis buffer (10 nM Tris-HCl (pH 7.5), 1 mM EDTA, 50 mM NaCl, 50 μg/ml PMSF) per g of wet cell paste and lysed by sonication (70-80% lysis based on OD). The lysate was clarified by centrifugation at 16,000 rpm in a JA-20 rotor for 45 minutes followed by heat treatment for one hour at 80° C. with occasional mixing. Heat precipitate was removed by centrifugation at 16,000 rpm in a JA-20 rotor for 60 minutes.

The supernatant was further clarified just before loading to the EMD SO₃ column by centrifuge in a JA-20 rotor at 16,000 rpm (˜20,000 g). The supernatant was loaded to 10 ml EMD-SO₃ column (1.6×5 cm) equilibrated with the low salt buffer (30 mM tris-HCl (pH 7.5), 50 mM NaCl, 1 mM EDTA, 1 mM DTT, 10% glycerol). The column was washed with 4 cv of the low salt buffer and eluted with 5 cv of a linear gradient over from the low buffer to 65% of the high salt buffer (30 mM tris-HCl (pH 7.5), 1000 mM NaCl, 1 mM EDTA, 1 mM DTT, 10% glycerol). 2.5 ml fractions were collected. The column was further eluted with 3 cv of 65% of the high salt buffer. Fractions were analyzed by SDS-PAGE (4-20% Novex Tris-Glycine gel) stained with Novex SimplySafe staining according to the manufacturer's manual. Fractions containing 17.5 kDa protein were pooled. The fraction pool was dialyzed against either 2 liter of the hydroxyapatite equilibration buffer (50 mM NaCl, 50 mM sodium phosphate (pH 6.8), 1 mM DTT, 5% glycerol) if the protein was purified from BL21(DE3), or the storage buffer (20 mM NaCl, 25 mM Tris-HCl, pH 7.5, 1 mM EDTA, 1 mM DTT, 10% glycerol) if from BL21-CodonPlus.

The sample dialyzed into the hydroxyapatite equilibration buffer was loaded to 2 ml Ceramic hydroxyapatite column (0.7×5.2 cm) equilibrated with the equilibration buffer. The column was washed with 10 cv of the equilibration buffer and eluted with 10 cv of a linear gradient from the equilibration buffer to the elution buffer (50 mM NaCl, 500 mM sodium phosphate (pH 6.8), 1 mM EDTA, 1 mM DTT, 5% glycerol) 1 ml fractions were collected. Fractions were analyzed by SDS-PAGE (4-20% Novex Tris-Glycine gel) stained with Novex SimplySafe staining. Fractions containing 17.5 kDa protein were pooled and dialyzed against the storage buffer.

Protein Assay for Purified AccuPrime Proteins: Bradford Protein Assay

Bradford protein assay was performed using Bio-Rad Protein Assay Dye Reagent Concentrate (Bio-Rad; Part# 500-0006) and lyophilized bovine gamma globulin (Bio-Rad; Part# 500-0005) reconstituted to 1.41 mg/ml as a standard. Three different measurements at three different dates were made to test reproducibility of the quantitation values.

QC Assays for Purified AccuPrime Protein II

Endo-nuclease activity. Endo-nuclease assay for a batch of AccuPrime protein II prep was performed using a double-stranded endonuclease assay. Each reaction contained 1 μg of supercoiled φX174 RF DNA and 4 (10×) or 8 (20×) μg of AccuPrime proteins in 50 μl of 1× Pfx Amplification buffer (18 mM (NH₄)₂SO₄, 60 mM Tris-SO₄, pH 8.9) with 1 mM MgSO₄. Reaction mix was incubated at 37° C. for 1 hr, and the reaction was terminated by adding 2.5 μl of 10% SDS and heating at 95° C. for 5 min. The reaction mix was assayed by agarose gel electrophoresis. The electrophoresis was done for 10 μl each of the mixes on a 0.8% horizontal agarose gel and the gel was stained with Ethidium Bromide.

Exo-nuclease activity. 100 pmol of oligonucleotide (36mer; 5′-GGG AGA CGG GGA ATT CGT CGA CGC GTC AGG ACT CTA-3′) was labeled with ³²P at the 5′ end using 10 units of T4 polynucleotide kinase and 10 μCi of [γ-³²P] ATP in 50 μl of 1× PNK exchange buffer. The reaction mix was incubated at 37° C. for 30 min and the reaction was terminated by incubating the mix at 70° C. for 10 min. Unincorporated nucleotides were removed by eluting the reaction mix through Amersham-Pharmacia Micro Spin G-25 column twice following the manufacturers instruction.

40 pmol of the radio-labeled oligonucleotide was incubated with 6 μg (10×) of AccuPrime protein in 100 μl of 1× Pfx Amplification buffer (18 mM (NH₄)₂SO₄, 60 mM Tris-SO₄, pH 8.9) with 1 mM MgSO₄ at 37° C. or 72° C. For samples incubated at 72° C., 20 μl aliquots were taken out at 0, 5, 10, and 30 min, and mixed with 10 μl of 3× formamide sequencing gel loading buffer and stored on ice. The samples were heated at 95° C. for 5 min and 10 μl each was loaded on a 15% TBE urea gel. For samples incubated at 37° C., 20 μl aliquots were taken out at 0, 15, 30, and 60 min, mixed with 1 μl each of 10% SDS and 20 mg/ml Proteinase K (Invitrogen; part# 25530-049), and incubated at 55° C. for 45 min. At the end of reaction, samples were mixed with 10 μl of 3× formamide sequencing gel loading buffer each and heated at 95° C. for 5 min. 10 μl each was loaded on a 15% TBE urea gel. The polyacrylamide gel was dried and autoradiographed using Kodak BioMax MR X-ray film.

Host DNA contamination. Host DNA contamination assay was done by PCR using a primer set targeting a single copy gene in E. coli genome (priA) in the presence of denatured AccuPrime Protein II at 1× (300 ng per 50 μl reaction) or 2× (600 ng) concentration without added DNA template. Denaturation of AccuPrime Protein II was accomplished by treating the protein solution (100 μl at 0.52 mg/ml) with 50 μg of proteinase K digestion at 55° C. for 1 hr. The peptidyl residues and the proteases were removed by extracting with phenol:chloroform:isoamyl alcohol (25:24:1) mix, followed by G-25 spin column (Pharmacia). The protein solution was treated as if it still contains the protein at the initial concentration for this purpose. Control reactions contain a known amount of E. coli genomic DNA in the absence of the protein as concentration markers in otherwise identical reactions. The E. coli priA 260 bp primer set was used: forward primer (priA_{—}260_{—F)} 5′-ACG CGC CGA TGT GOT ACT GGT TT-3′; reverse primer (priA_{—}260_R) 5′-GCG GTG GCC TGT TCG GTA TTC AA-3′.

E. coli DNA concentration control reaction contained either 0.1, 0.5 or 1 ng of genomic DNA from E. coli BL21 strain. PCR reaction was done with either Platinum Pfx DNA polymerase or Platinum Taq DNA polymerase in respective reaction conditions. To inactivate the enhancing activity of AccuPrime protein II on PCR reaction the protein was digested with proteinase K. Proteinase K digestion was done in 50 μl reaction containing 20 μg of AccuPrime protein II and 20 μg of proteinase K at 55° C. for 1 hour. Proteinase K was subsequently removed from the protein by phenol extraction and G-50 spin column. Proteinase treated AccuPrime protein solution was used as if it contained the protein at the protein concentration the same as that prior to the protease treatment for this purpose. The reaction mix was assayed by agarose gel electrophoresis. The electrophoresis was done for 10 μl each of the mixes on a 0.8% horizontal agarose gel and the gel was stained with Ethidium Bromide.

Functional PCR Assay

Functional PCR assay was performed to establish functionality of the purified AccuPrime Protein II. The assay was done using p53 2380 primer set with 100 ng of human genomic DNA (K562, genotyping grade) in 50 μl reactions except the increasing amount of AccuPrime Protein II from 100 to 600 ng per reaction at the increment of 100 ng, in the presence of 100 ng of AccuPrime Protein I. The Human p53 2380 bp primer set was used: forward primer (p53_{—}2380_F) 5′-CCC CTC CTG GCC CCT GTC AT-3′; reverse primer (p53_{—}2380_R) 5′-GCA GCT CGT GGT GAG GCT CCC-3′.

Platinum Pfx DNA polymerase in 1× Pfx amplification buffer with 1 mM MgSO4 was used in control reactions. PCR reaction was performed as following:

PCR program for functional Assay:

Pre-incubation

| | |
|---|---|
| 95° C. | 5 minutes |
| 35 cycles of | |
| 95° C. | 15 seconds |
| 63° C. | 30 seconds |
| 68° C. | 3 min |
| Hold at 4° C. | |

Following the completion of thermocycling, PCR amplification products were mixed with 5 μl of 10× BlueJuice and aliquot (20% of total reaction volume, or 10 μl, per each lane) were analyzed on 0.8% agarose gel electrophoresis with an ethidium bromide concentration of 0.4 μg/ml premixed in 0.5×TBE. The resulting gels were analyzed visually for specificity and yield among different samples.

Stability of AccuPrime Protein in AccuPrime Formulation

Accelerated stability assay. Accelerated stability assay is based on assumption that an elevated temperature would thermodynamically accelerate the rate of a reaction, and that deterioration (inactivation, denaturation or degradation) of a protein is a reaction from the thermodynamic point of view. Therefore, incubating a protein solution at a higher temperature for a certain period of time would mimic an effect of a longer period of storage at a lower temperature. The extent of the acceleration was estimated using Arrhenius equation k=A e^{[·Ea/R T]}, where k is the rate constant, A is a constant, Eₐ is the activation energy, R is the universal gas constant 8.314×10⁻³ kJ mol⁻¹K⁻¹, and T is the temperature (in degrees Kelvin).

10× AccuPrime Pfx reaction mix was tested at 37 and 45° C. for 7 and 4 days, respectively, which were equivalent to 1 yr of storage at −20° C. After the period of incubation, the reaction mix (or Supermix) was tested for its function using PCR at 1× strength. For the functional assay, a primer set was selected for its difficulty in its PCR in obtaining specific product with other Pfx DNA polymerases. The Human β globin (Hbg) 3.6 kb primer set was used: forward primer (Hbg_{—}3.6_{—F)} 5′-TTC CTG AGA GCC GAA CTG TAG TGA-3′; reverse primer (Hbg_{—}3.6_R) 5′-TAA GAC ATG TAT TTG CAT GGA AAA CAA CTC-3′.

The intensity of the specific band was to be enhanced by functional AccuPrime Pfx at 3.6 kb in length. PCR reaction was carried out in a standard manner using 50 ng of K562 genotyping DNA as template and 0.3 μM each of the primers in 50 μl of 1× Pfx amplification buffer including 1 mM MgSO₄. PCR incubation was set with 95° C. pre-incubation for 5 min, followed by 35 cycles of 95° C. for 15 sec, 62° C. for 30 sec and 68° C. for 4 min. The PCR products were analyzed on a 0.8% horizontal agarose gel.

Real-time stability assay. Real-time stability assay was performed similarly to the accelerated stability assay with a few exceptions. This time all the formulation contained nucleotides. The lot for 10× reaction mix formulation was divided to two batches to include one batch without glycerol. Incubation was done at three different temperatures, −20, 4 and 22° C.

The criterion for the PCR functional assay was same as above in that a functional reaction mix would enhance the specific band of 3.6 Kb in length for the Hbg 3.6 primer set. PCR reaction was carried out in a standard manner using 20 ng of K562 genotyping DNA as genomic template or 200 fg of pUC19 as cDNA template and 0.2 μM each of the primers in 50 μl of 1× PCR buffer. PCR incubation was set with 95° C. pre-incubation for 5 min, followed by 35 cycles of 95° C. for 15 sec, 62° C. for 30 sec and 68° C. for 4 min. The PCR products were analyzed on a 0.8% horizontal agarose gel.

PCR Reactions for AccuPrime Pfx DNA Polymerase

Standard PCR reactions. Unless otherwise indicated, all the PCR reactions were run following a standard protocol. PCR reactions were prepared in 50 μl reaction volumes containing 1× Pfx Amplification buffer (18 mM (NH₄)₂SO₄, 60 mM Tris-SO₄, pH 8.9) with 1 mM MgSO₄, and 0.3 μM of each primer. The concentration of each of four deoxynucleoside triphosphate (dNTPs) was 0.3 mM. 1× AccuPrime Pfx reaction mix contains 100 ng AccuPrime Protein I and 300 ng AccuPrime Protein II per 50 μl reaction in addition. Template concentration varied from 20 ng to 200 ng depending on the application. One unit of AccuPrime Pfx DNA polymerase were used in a typical 50 μl reaction. Thermocycling was conducted using either Perkin Elmer GeneAmp PCR System 9600, Perkin Elmer GeneAmp PCR System 2400 or MJR Peltier Thermal Cycler (PTC) 200.

Standard PCR Program:

Pre-incubation

| | |
|---|---|
| 95° C. | 5 minutes |
| 35 cycles of | |
| 95° C. | 15 seconds |
| 55° C.-65° C. | 30 seconds (5 degrees below Tm) |
| 68° C. | 1 min/kb |
| Hold at 4° C. | |

Following the completion of thermocycling, PCR amplification products were mixed with 5 μl of 10× BlueJuice and aliquot (20% of total reaction volume, or 10 μl, per each lane) were analyzed on 0.8%-1.5% agarose gel electrophoresis with an ethidium bromide concentration of 0.4 μg/ml premixed in 0.5×TBE. The resulting gels were analyzed visually for specificity and yield among different samples.

rpsL Fidelity Assay

Fidelity assay was performed based on streptomycin resistance of rpsL mutation exhibits (Lackovich et al., 2001; Fujii et al., 1999). Briefly, pMOL 21 plasmid DNA (4 kb), containing the ampicillin (Ap′) and (rpsL) genes, was linearized with Sca I and standard PCR was performed on the linearized product using biotinylated primers. Amplification was completed using 2 units of AccuPrime Pfx DNA polymerase. Template DNA was 1 ng for 25 cycles of amplification. PCR cycling parameters were 95° C. for 5 min, followed by 25 cycles of 95° C. for 15 s, 58° C. for 30 s, and 68° C. for 5 min. PCR product was streptavidin-magnetic-bead-purified to ascertain linearity. Purified PCR product was analyzed on an agarose gel, and DNA concentration and template doubling was estimated. The purified DNA was ligated with T4 DNA ligase and transformed into MF101 competent cells. Cells were plated on ampicillin plates to determine the total number of transformed cells. Cells were plated on ampicillin and streptomycin plates to determine the total number of rpsL mutants. Mutation frequency was determined by dividing the total number of mutations by the total number of transformed cells. The error rate was determined by dividing the mutation frequency by 130 (the number of changes in amino acid sequence that cause phenotypic changes for rpsL) and the template doubling.

Competitive Audit of AccuPrime Pfx DNA Polymerase

Performance of AccuPrime Pfx DNA polymerase was compared with competitive high-fidelity PCR enzymes, such as Pfu Turbo DNA Polymerase (Stratagene, Cat. No. 600252, lot 1210608), Pwo DNA Polymerase (Roche, Cat. No. 1644 955, lot 49215324), Tgo DNA Polymerase (Roche, Cat. No. 3186 199, lot 90520522), and KOD Hot Start DNA Polymerase (Novagen, Cat. No. 71086-3, lot N33243). Each enzyme was used to amplify targets ranging from 822 bp to 6816 bp using 100 to 200 ng of human genomic DNA (K562, genotyping grade). Primers and their sequences are as follows: (#1, c-myc 822 bp primer set) forward primer (cmyc_{—}822_F) 5′-CGG TCC ACA ACG TCT CCA CTT-3′, reverse primer (cmyc_{—}822_R) 5′-CTG TTT GAC AAA CCG CAT CCT TG-3′; (#2, p53 2380 bp primer set) forward primer (p53_{—}2380_F) 5′-CCC CTC CTG GCC CCT GTC AT-3′, reverse primer (p53_{—}2380_R) 5′-GCA GCT CGT GGT GAG GCT CCC-3′; (# 3, Human β globin (Hbg) 3.6 kb primer set) forward primer (Hbg_{—}3.6_F) 5′-TTC CTG AGA GCC GAA CTG TAG TGA-3′, reverse primer (Hbg_{—}3.6_R) 5′-TAA GAC ATG TAT TTG CAT GGA AAA CAA CTC-3′; (#4, Rhod 6173 bp primer set) forward primer (Rhod_{—}575_F) 5′-CCC TCT ACA CCT CTC TGC ATG GA-3′, reverse primer (Rhod_{—}6748_R) 5′- AGC AAC AAA ACC CAC CAC CGT TA-3′; (#5, Rhod 6816 bp primer set) forward primer (Rhod_{—}532_F) 5′- GCC GTG GCT GAC CTC TTC ATG GT-3′, reverse primer (Rhod_{—}6748_R) 5′- AGC AAC AAA ACC CAC CAC CGT TA-3′.

PCR reactions were performed following manufacturers' recommendation as closely as practically possible. Annealing temperature for each primer set was set identically for all the polymerases tested, which are: 65° C. for c-myc 822 bp and p53 2380; 62° C. for bp (Hbg) 3.6 kb; and 64° C. for Rhod 6173 bp Rhod 6816 bp. Detailed PCR conditions are as follows:

PCR program for Pfu Turbo and Tgo DNA Polymerases:

Pre-incubation

| | |
|---|---|
| 95° C. | 5 minutes |
| 35 cycles of | |
| 95° C. | 30 seconds |
| 62° C.-65° C. | 30 seconds (see above) |
| 72° C. | 1 min/kb |
| Hold at 4° C. | |

PCR program for Pwo DNA Polymerase:

Pre-incubation

| | |
|---|---|
| 94° C. | 2 minutes |
| 10 cycles of | |
| 94° C. | 15 seconds |
| 62° C.-65° C. | 30 seconds (see above) |
| 72° C. | 2 min |
| 25 cycles of | |
| 94° C. | 15 seconds |
| 62° C.-65° C. | 30 seconds (see above) |
| 72° C. | 2 min + 5 sec increase per each cycle. |

Post-cycle incubation

| | |
|---|---|
| 72° C. | 7 min |
| Hold at 4° C. | |

PCR program for KOD Hot Start and AccuPrime Pfx DNA Polymerases:

Pre-incubation

| | |
|---|---|
| 95° C. | 5 minutes |
| 35 cycles of | |
| 95° C. | 15 seconds |
| 62° C.-65° C. | 30 seconds (5 degrees below Tm) |
| 68° C. | 1 min/kb |
| Hold at 4° C. | |

PCR amplification products were mixed with 5 μl of 10× BlueJuice and aliquot (20% of total reaction volume, or 10 μl, per each lane) were analyzed on 0.8% agarose gel electrophoresis with an ethidium bromide concentration of 0.4 μg/ml premixed in 0.5×TBE. The resulting gels were analyzed visually for specificity and yield among different samples.

### RESULTS AND DISCUSSION

Purification of AccuPrime Protein II

The clone of AccuPrime Protein II was received from Dr. Steve Kowalczykowski's lab at UC Davis in BL21(DE3) CodonPlus strain, due to frequent use of rare codons in the gene. The original purification protocol asked for room temperature purification, once heat treatment at 80° C. for 1 hour was finished. The protocol called for ssDNA cellulose column followed by Resource Q column (Pharmacia) chromatographies, which would yield about 0.8 mg of the protein from 1 liter culture. Several trial of the purification in our hand following the process unfortunately resulted in very poor yield, mainly due to poor retention of the protein in either of the two columns.

Dr. Kowalczykowski and his colleagues (Haseltine and Kowalczykowski, 2002) reported that the dissociation constant for single-stranded DNA was around 0.5 μM or 3 order of magnitude higher than that of AccuPrime protein I. This result explains the poor retention of the protein in the ssDNA cellulose column.

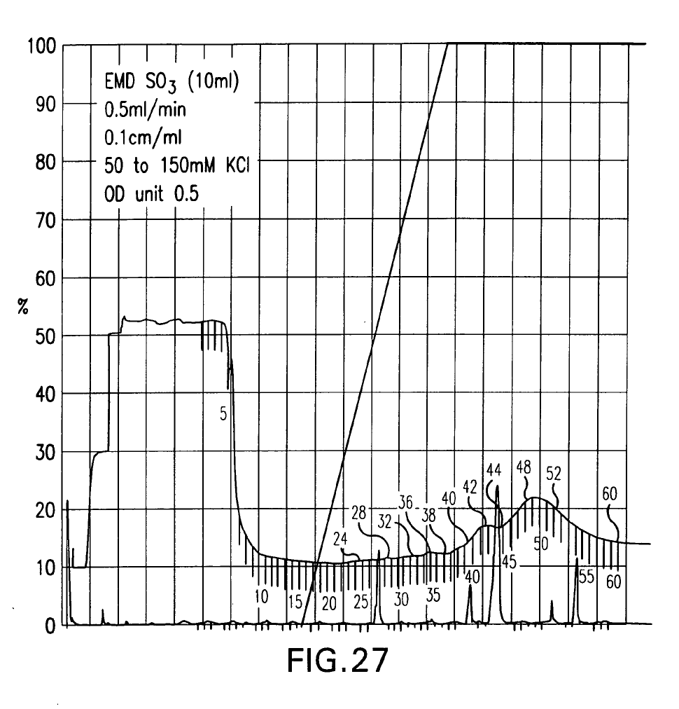

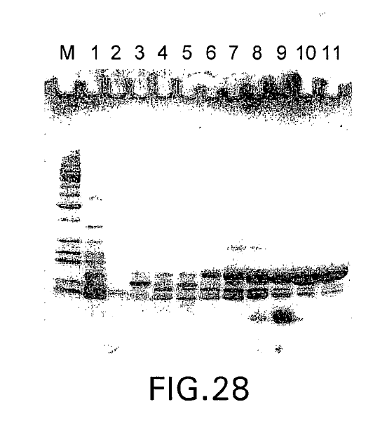

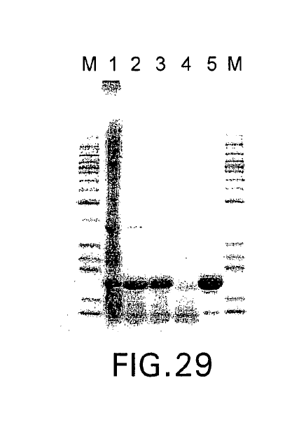

To improve the yield, Fractogel EMD-SO₃ column chromatography was introduced to the purification protocol in the place of ssDNA column chromatography. FIGS. 27 and 28 show elution profile and cross-column analysis on SDS-PAGE, respectively. The results show almost complete removal of any contaminating proteins from AccuPrime Protein II by the single column chromatography step. FIG. 29 shows again the plausibility of one step purification of the protein, with increased yield to 3 to 4 times higher than the original protocol (25 mg of the purified protein from 10 liters of culture, compared to 8 mg from the same volume of culture using the original protocol).

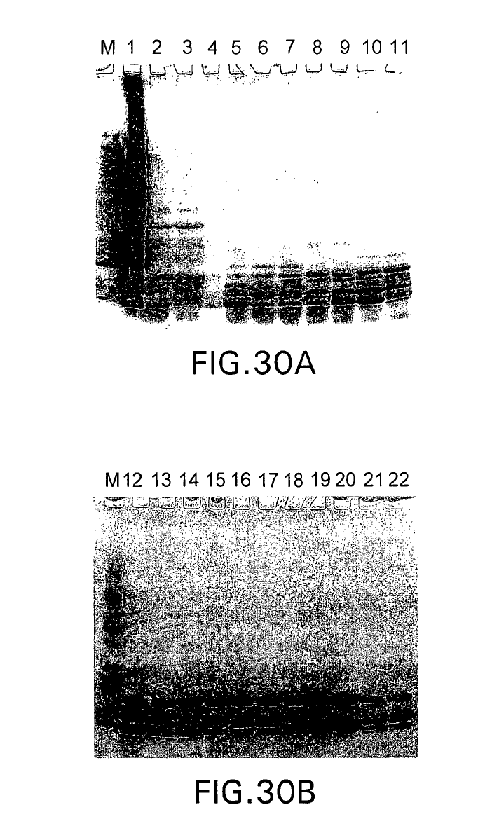

On the other hand, the protein purified from BL21(DE3) strain via EMD-SO₃ showed a higher contamination with smaller peptides (FIG. 30). An effort to separate those contaminants by MonoQ column failed since neither the protein nor the contaminants were retained by the column (data not shown). There is a possibility that those contaminants might be product of early termination products during translation due to the frequent rare codons in the gene.

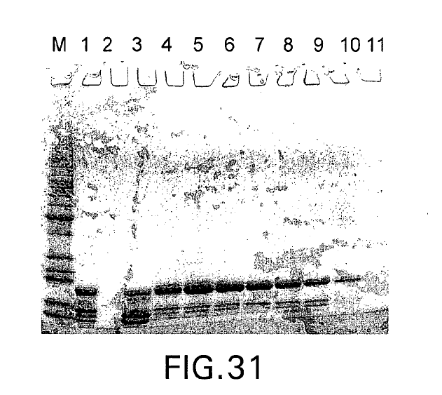

Majority of the contaminants were eluted out from the Hydroxyapatite column (BioRad, Bio-Scale CHT2-1 hydroxyapatite column, type I, 2 ml, Batch# 74603) during the washing step with 50 mM Na phosphate buffer (pH 6.8) while AccuPrime Protein II was eluted in the gradient step at the phosphate concentration around 200 mM (FIG. 31). The purity was estimated about 85% with the yield at 5 mg of AccuPrime Protein II from 50 gram of wet cell (about 20 liter culture equivalent).

We converted the rare codons to more common codons using overlapping synthetic oligonucleotides. The new construct brought back one step purification with EMD-SO₃ column for higher yield and high purity similar to those purified from BL21 (DE3) CodonPlus host (FIG. 29).

Protein Assay for AccuPrime Protein II

Table 11 shows several repeats of Bradford protein assays, Standard assay or microassay, using a single protein stock solution (lot 2002-50-67). The standard deviation for Bradford Standard assay was about 4 times higher than that of the microassay, while the micro assay showing about 50% higher value for the concentration, which seemed to correlate more with band intensity of SDS-PAGE.

TABLE 11

| Assay | Standard Bradford | Bradford microassay |
|---|---|---|
| First | 0.63 | 0.90 |
| Second | 0.85 | 0.95 |
| Third | 0.42 | |
| Fourth | 0.59 | |
| Fifth | 0.62 | |
| Sixth | 0.69 | |
| Deviation | 0.63 ± 0.140 | 0.93 ± 0.035 |

It is known for Bradford assay to be fluctuating, however, the higher standard deviation from the Standard method make it less reliable than the microassay. The results clearly show the inherent problem associated with protein assay methods using chromogenic dyes, such as Bradford assay. Bradford assay measures an extensive property of the solution (the volume of the sample solution added to the dye solution, in addition to the concentration, determines the outcome). This result seems to indicate that the standard assay may be more susceptable to variation of the condition where the assay was performed. As a result, it was decided to follow the Microassay for protein assay for AccuPrime Protein II.

The protein concentration for the new batch of AccuPrime Protein II from BL21(DE3) (lot 2002-132-41) was determined to be 0.52 mg/ml, which resulted in total of 5 mg (see above).

QC Assays for AccuPrime Protein

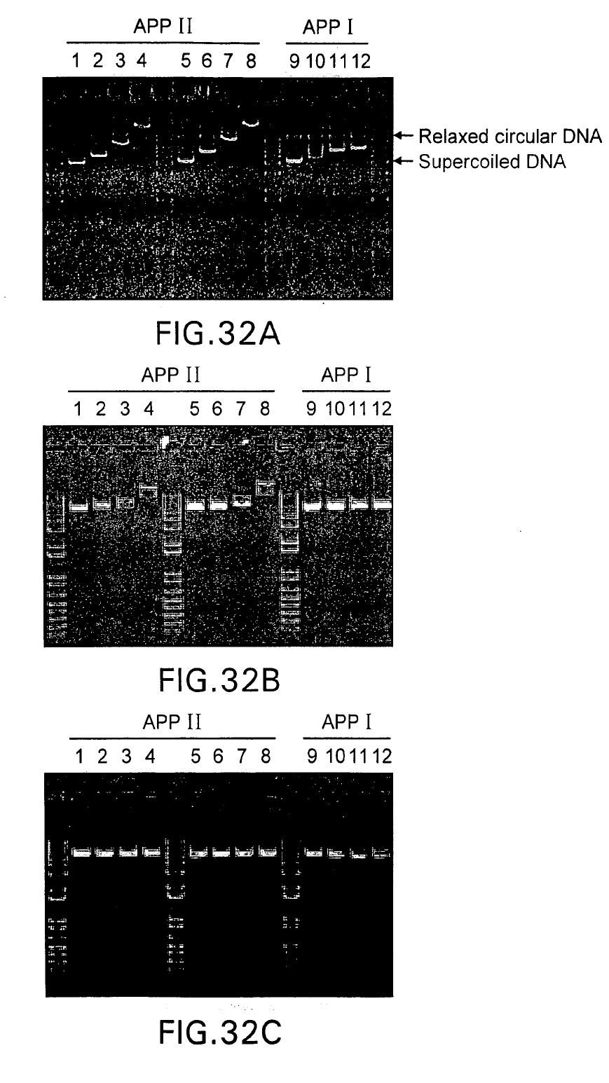

Endo-nuclease activity. No detectable endonuclease activity was found in the AccuPrime protein prep at the protein concentration up to 10× of that recommended for PCR reaction using a double stranded, super-coiled substrate (FIG. 32). After hour incubation at 37° C., the band pattern did not change from that of control that was incubated under identical condition except the absence of the protein (lanes 1, 5 and 9 from Panel C, FIG. 32). This result indicates that not even a nicking activity was found in the prep.

However, what was observed was a mobility shift on samples that were not treated with heat and SDS (Panel A in FIG. 32). Since the heat treatment in presence of SDS recovered the mobility, it is as a result of protein binding to the DNA template. A similar result was shown with AccuPrime protein I (lanes 9 to 12, FIG. 32). However, unlike the AccuPrime protein I binding where the DNA bands were always between supercoil and relaxed circles, AccuPrime protein II binding results in mobility shift that was slower than a relaxed circular DNA.

A strong ssDNA binding activity may cause unwinding of dsDNA especially with negatively supercoiled DNA. Such unwinding would create pseudo-topoisomers with a lower gel mobility and the mobility would never be slower than relaxed circular DNA as shown with AccuPrime Protein I. AccuPrime Protein II has lower binding affinity to ssDNA than AccuPrime Protein I by about 3 order of magnitude. Considering those facts, the shift may come from dsDNA binding of AccuPrime protein II.

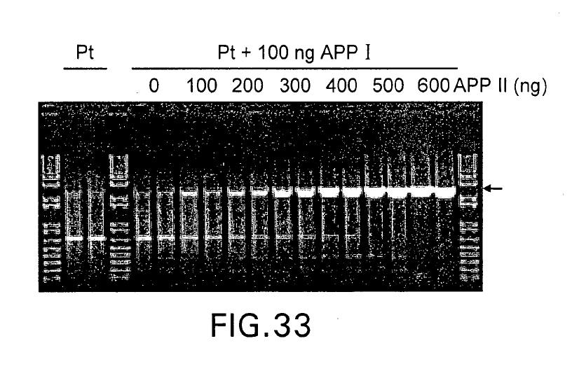

PCR functional Assay. Functionality of the purified AccuPrime Protein II was assayed by its ability to enhance PCR reaction with Platinum Pfx DNA polymerase in a concentration dependent manner in the presence of 100 ng of AccuPrime Protein I per 50 μl reaction (FIG. 33). PCR reaction using p53 2380 bp primer set (see Materials and Methods) was done otherwise standard Platinum Pfx DNA polymerase condition in the presence or the absence of AccuPrime proteins as indicated in the Figure legend.

The results indicate that the addition of AccuPrime Protein II enhanced the yield of the specific 2380 bp long product while suppressing non-specific product in a concentration dependent manner all the way up to 600 ng of the protein. However, it was observed earlier that for some primer sets, a higher concentration of AccuPrime Protein than 300 ng per 50 μl reaction was inhibitory.

Host DNA contamination assay. There is always a possibility when a protein with DNA binding activity was purified from an organism, the protein might have been co-purified with some host genomic DNA, still bound to it. Since PCR is a strong technique in amplifying even a minute quantity of DNA, it is always a concern that PCR related proteins be free of host DNA contamination.

AccuPrime Protein II was tested for the presence of host DNA contamination in each batch. Host DNA contamination was tested using PCR reaction with primer set targeting a single-copy gene in the genome of E. coli where the protein was expressed and purified from. Host DNA contamination assay was done by PCR using a primer set targeting a single copy gene in E. coli genome (priA) in the presence of denatured AccuPrime Protein II at 1× (300 ng per 50 μl reaction) or 2× (600 ng) concentration without added DNA template. Control reactions contain a known amount of E. coli genomic DNA in the absence of the protein as concentration markers in otherwise identical reactions.

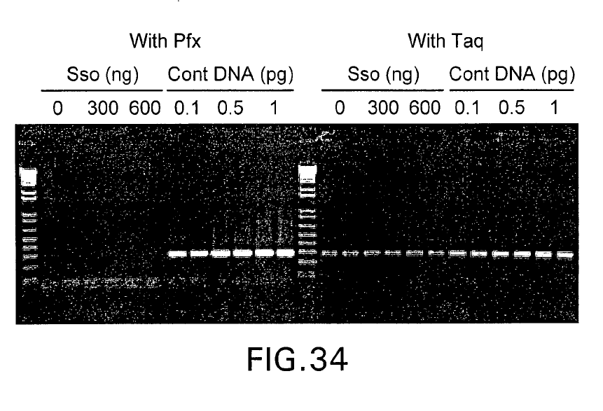

No specific band was observed in reactions containing Pfx DNA polymerase (FIG. 34). However, a specific band was observed in reactions containing Taq DNA polymerase, even in the control lane. The band intensity did not increase with increasing amount of protein, indicating that the source of the contaminating DNA was not AccuPrime Protein II but rather the polymerase.

PCR Application Development for AccuPrime Pfx DNA Polymerase

Optimization of AccuPrime Pfx DNA polymerase. It was intended that AccuPrime Pfx DNA polymerase be formulated similarly to AccuPrime Taq DNA polymerase in that it would consist of Platinum Pfx DNA polymerase and AccuPrime Protein I.

The initial formulation containing just AccuPrime Protein I with a lower Antibodies to enzyme ratio (Ab1:Ab2:Pfx=2:2:1, instead of 5:5:1 as in Platinum Pfx DNA polymerase), or “Formula A,” was proven to be less than optimal in its ability to enhance the performance of Pfx DNA polymerase as summarized in Table 12.

TABLE 12

| Primers | Marked | Slight | Already | No | |
|---|---|---|---|---|---|
| tested | improvement | improvement | good | improvement | Inhibited |
| 71 | 8 | 12 | 18 | 32 | 1 |
| | (11%) | (17%) | (25%) | (45%) | (1%) |

The updated formulation, or “Formula B,” would contain two different single stranded DNA binding proteins from Methanococcus janaschii and Sulfolobus solfataricus, or MjaSSB (AccuPrime Protein I, 1, 100 ng per 50 μl reaction) and SsoSSB (AccuPrime Protein II, 300 ng), respectively. In combination with the lower antibody contents, the new formulation was proven to be very robust with a higher specificity and sensitivity (see below).

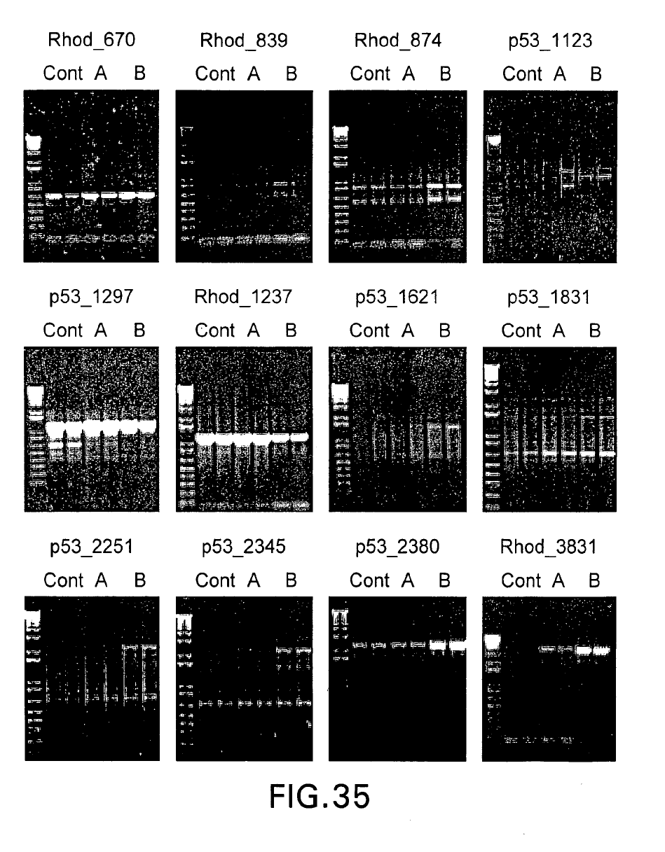

In performance comparison using 35 primer sets with amplicons ranging from 504 bp to 4.4 kb, the new AccuPrime Pfx formula (Formula B) with both AccuPrime Protein I and AccuPrime Protein II out performed Platinum Pfx DNA polymerase and even the old AccuPrime Pfx formula (Formula A) (FIG. 35). The statistical analysis of the results are shown in Table 13.

TABLE 13

| Primers | Marked | No | |
|---|---|---|---|
| tested | improvement | improvement | Inhibited |
| 35 | 15 | 19 | 1 |
| | (43%) | (54%) | (3%) |

AccuPrime Pfx DNA polymerase formulation was finalized with the Formula B containing both AccuPrime Protein I and AccuPrime Protein II with the lower antibodies to the enzyme ratio (Ab1:Ab2:Pfx=2:2:1), and AccuPrime Pfx DNA polymerase this point on will be referred to the Formula B.

It was observed, however, that enhancement by AccuPrime Pfx DNA polymerase was shown mostly with the primer sets targeting amplicon sizes less than 3 kb. This does not mean in any way to say that AccuPrime Pfx would not work on targets longer than 3 kb, but its performance with the longer target is at least of the level of Platinum Pfx which was previously shown to amply targets as long as 12 kb. Table 14 shows the impressive enhancement by AccuPrime Pfx DNA polymerases with amplicons shorter than 3 kb.

TABLE

| Primers | Marked | No | |
|---|---|---|---|
| tested | improvement | improvement | Inhibited |
| 18 | 14 | 4 | 0 |
| | (78%) | (22%) | (0%) |

In summary, AccuPrime proteins enhance Pfx DNA polymerase in its sensitivity and specificity. However, it was unexpected to see combined effect of the two SSB proteins from different origins would be larger than the sum the effects from individual proteins. Previous study on AccuPrime Taq DNA polymerase system revealed that MjaSSB (AccuPrime Protein I) enhanced the specificity of Taq DNA polymerase by preventing primers from annealing non-specifically, acting as a competitive inhibitor against non-specifically annealed primers, and recruiting the polymerase to the specifically primed sites, increasing the local concentration of the enzyme where it is needed. In several cases with different polymerases, AccuPrime Protein II makes PCR enzymes very robust in increasing the yield of the PCR products, sometimes even non-specific ones as well (the result will be reported elsewhere), definitely functioning in a different manner from that of AccuPrime Protein I.

It is a novel finding that a SSB could function differently from another SSB despite their homology and the significant sequence conservation throughout the evolution. It is even unique that two differently functioning SSB proteins complement each other in enhancing the performance of PCR enzymes. That makes AccuPrime Pfx DNA polymerase the first PCR enzyme benefiting from such novel properties.

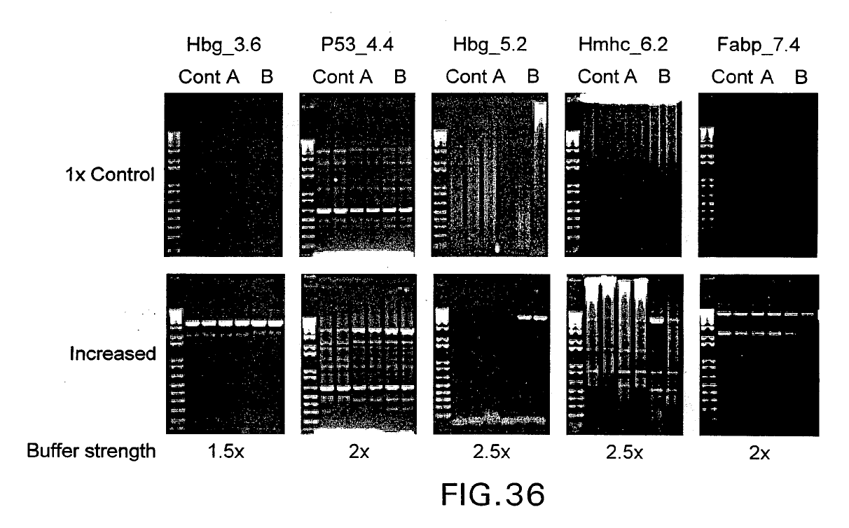

It was found that a higher concentration of Pfx amplification buffer in a reaction enhanced the activity of Platinum Pfx DNA polymerase in PCR. The observation was confirmed by set of PCR reactions by AccuPrime Pfx DNA polymerase with amplicons sized ranging from 3.6 Kb to 7.4 Kb (FIG. 36). However, 3× buffer concentration was proven to be inhibitory.

In all most all cases tested, a higher buffer concentration enhanced overall performance of Pfx DNA polymerase, in general. It seems also true that in some cases, the enhancing effect was higher in one formula of Pfx than the other depending on the primers used. But even in such cases the lesser enhancement was an enhancement from the result with 1× buffer concentration. However, titrating the buffer concentrations seem necessary for the best result. Based on this result, it was decided to keep 1× concentration in final formulation of AccuPrime Pfx Reaction Mix, where we could assure a consistent performance.

Since AccuPrime Pfx reaction mix will contain all the buffer components and there will be no separate amplification buffer provided with the kit, it would be hard to recommend the titration of the buffer concentration as an option for PCR optimization. It was probed to see if any of the buffer components or an additional factor would result in similar enhancing effect. For that, ammonium sulfate and potassium chloride were tested.

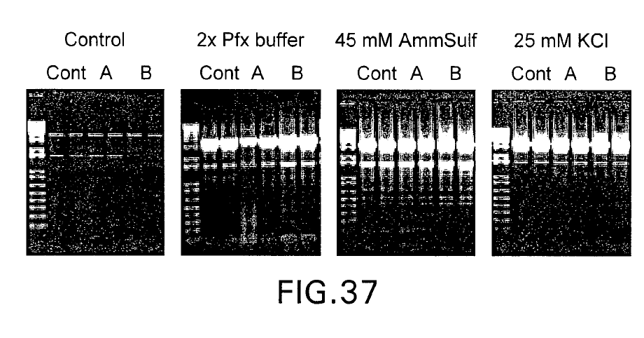

A set of PCR reaction was performed using Hbg_{—}3.6 primer set. The primer set is currently used in functional QC assay for Platinum Pfx DNA polymerase and most likely will be for AccuPrime Pfx as well. As seen in first panel in FIG. 36, the amplification from the primer set was not always an easy task. However, all the optimization options used show enhancements. The optimization options used are 2× Pfx amplification buffer, 2.5× of ammonium sulfate or 40 mM KCl. The result in FIG. 37 indicated at least for this primer set, additional ammonium sulfate or potassium chloride in the reaction mix showed equal or better enhancement in Pfx performance. Several other primer sets testes with KCl also proved that titration of KCl for PCR optimization would be a viable option (data not shown).

Comparison Audit of AccuPrime Pfx DNA Polymerase Against Other High-Fidelity PCR Enzymes

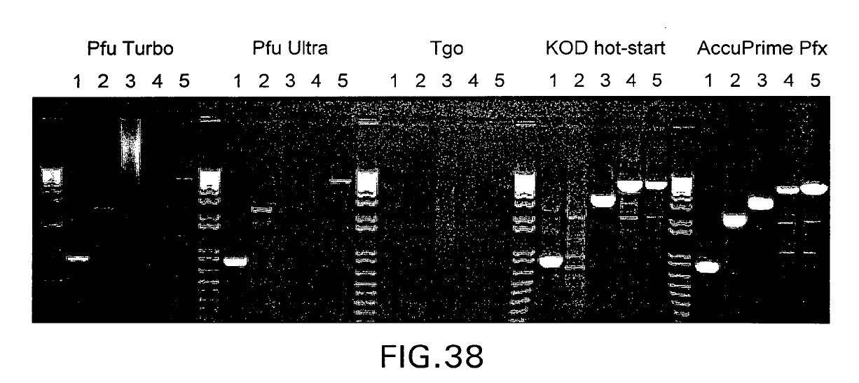

In performance comparison using 5 primer sets with amplicons ranging from 822 to 6,816 bp (c-myc 822 bp; p53 2380 bp; Hbg 3.6 kb; Rhod 6173 bp; and Rhod 6816 bp), Performance of AccuPrime Pfx DNA polymerase was directly compared with other major high-fidelity polymerases, such as, Pfu Turbo DNA Polymerase (Stratagene), Pfu Ultra DNA Polymerase (Stratagene), Tgo DNA Polymerase (Roche), and KOD Hot Start DNA Polymerase (Novagen). AccuPrime Pfx DNA polymerase showed a superior performance over all others tested in the yield, the specificity, robustness and consistency (FIG. 38).

We matched Stratagene's Pfu Turbo DNA polymerase performance with Platinum Pfx DNA polymerase and certainly surpassed with AccuPrime Pfx DNA polymerase.

### CONCLUSION

We report here development of another member of the next generation PCR enzyme family, namely AccuPrime Technology. AccuPrime Taq DNA polymerase system and its companion AccuPrime Taq PCR SuperMix incorporate a thermostable AccuPrime protein to the Platinum technology that enhances the performance of Taq DNA polymerase in PCR beyond Platinum Taq DNA polymerase. The enhancement is shown in all aspects of PCR performance of the enzyme such as the specificity, sensitivity, and robustness. The enhancing factor for the system is AccuPrime Protein I, or thermostable SSB from Methanococcus jannaschii. Since the nature designed SSB to help DNA polymerase in all living organisms in replication, we expected its enhancing effect on a DNA polymerase. Such enhancement makes AccuPrime Taq DNA polymerase system suitable to many areas of PCR application, such as, high throughput PCR, multiplex PCR and PCR miniaturization.

It is only fitting that we apply the technology to the most finicky PCR application of all, the high fidelity DNA polymerase. Due to the very nature of high fidelity DNA polymerases where the enzyme has a proofreading, or 3′-5′ exonuclease, activity in addition to the polymerase activity. These counteracting activities of the polymerases make them useful but difficult to harness in PCR application. The challenge was whether we could apply AccuPrime Technology to such finicky enzymes. As it turned out, we had to modify the AccuPrime Technology to accommodate Pfx DNA polymerase, a high-fidelity DNA polymerase. The modification includes the addition of a second SSB, SSB from Sulfolobus solfataricus, or AccuPrime Protein II. It is a surprise finding that a pair of supposedly functionally homologous proteins could complement each other in enhancing the polymerase. The complementary actions of the AccuPrime proteins may be derived from different characteristics of the two SSB, such as, their quaternary structures, and ssDNA and dsDNA binding affinities. With the modified AccuPrime Technology AccuPrime Pfx DNA polymerase becomes most robust high fidelity DNA polymerase with higher specificity and sensitivity than all other high fidelity enzymes in the current market.

System configurations are provided in Table 15.

TABLE 15

| Name | Components |
|---|---|
| AccuPrime Pfx PCR | 2 units/ul AccuPrime Taq (2 units/ul rTaq DNA |
| DNA polymerase | polymerase, 40 ng/ul each of two anti-Taq DNA |
| | polymeras antibodies; 1:5 Molar ratio) 50 mM |
| | MgCl₂ |
| 10x AccuPrime Pfx | 10X AccuPrime Pfx Reaction mix with |
| Reaction Mix | AccuPrime ™ proteins {10× Pfx Amplification |
| | buffer [600 mM Tris-SO₄ (pH 8.9), 180 mM |
| | (NH₄)₂SO₄], 10 mM MgCl₂, 3 mM dGTP, 3 mM |
| | dATP, 3 mM dTTP, 3 mM dCTP, 20 ug/ml |
| | of MjaSSB, 60 ug/ml of SsoSSB} |

### REFERENCES

Chedin, F, Seitz, E. M. and Kowalczykowski, S. C. (1998). “Novel homologs of replication protein A in archaea: implications for the evolution of ssDNA-binding proteins.” Trends Biochem. Sci. 23, 273-277
Chou, Q. (1992). “Minimizing deletion mutagenesis artifact during Taq DNA polymerase PCR by E. coli SSB.” Nucl. Acids Res. 20, 4371
Dabrowski, S. and Kur, J. (1999). “Cloning, overexpression, and purification of the recombinant His-tagged SSB protein of Escherichia coli and use in polymerase chain reaction amplification.” Protein Expr. Purif. 16 (1), 96-102
Fujii, S., Akiyama, M., Aoki, K., Sugaya, Y., Higuchi, K., Hiraoka, M., Miki, Y., Saitoh, N., Yoshiyama, K., Ihara, K., Seki, M., Ohtsubo, E. and Maki, H. (1999). “DNA replication errors produced by the replicative apparatus of Escherichia coli.” J. Mol. Biol. 289, 835-850
Glover, B. P. and McHenry, C. S. (1998). “The χψ subunits of DNA polymerase III holoenzyme bind to single-stranded DNA-binding protein (SSB) and facilitate replication of an SSB-coated template.” J. Biol. Chem. 273, 23476-23484
Haseltine, C. A. and Kowalczykowski, S. C. (2002). “A distinctive single-stranded DNA-binding protein from the Archaeon Sulfolobus solfataricus.” Mol. Microbiol. 43, 1505-1515

Kelly, T. J., Simancek, P. and Brush, G. S. (1998). “Identification and characterization of a single-stranded DNA-binding protein from the archaeon Methanococcus jannaschii.” Proc. Natl. Acad. Sci. USA 95, 14634-14639
Kim, Y. T., Tabor, S., Churchich, J. E. and Richardson, C. C. (1992). “Interactions of gene 2.5 protein and DNA polymerase of bacteriophage T7.” J. Biol. Chem. 267, 15032-15040
Kim, Y. T. and Richardson, C. C. (1994). “Acidic carboxyl-terminal domain of gene 2.5 protein of bacteriophage T7 is essential for protein-protein interactions.” J. Biol. Chem. 269, 5270-5278
Lackovich, J. K., Lee, J. E., Chang, P. and Rashtchian, A. (2001). “Measuring fidelity of Platinum Pfx DNA polymerase.” Focus 23, 6-7
Lee, J., Chastain, P. D., Jr., Kusakabe, T., Griffith, J. D. and Richardson, C. C. (1998). “Coordinated leading and lagging strand DNA synthesis on a minicircular template.” Mol. Cell. 1, 1001-1010
Rapley, R. (1994). “Enhancing PCR amplification and sequencing using DNA-binding proteins.” Mol. Biotechnol. 2, 295-298

### Example 6

### Enhancement of Thermalace™ DNA Polymerase Performance by SSBS Individually and in Combination

ThermalAce™ DNA polymerase (Invitrogen Corp.) is a thermostable archaebacterial enzyme having high processivity and 3′ to 5′ exonuclease proofreading activity (see U.S. Pat. No. 5,972,650). PCR was performed using ThermalAce™ DNA polymerase in conjunction with M. jannachii SSB (MjaSSB), M. thermoautotrophicum SSB (Mth SSB), and S. solfataricus SSB (SsoSSB). PCR reactions included 1-100 ng DNA template (K562 human genomic DNA, genotyping grade), 100 ng of each amplification primer (Rhod_{—}147F: 5′-AGG AGC TTA GGA GGG GGA GGT-3′ and Rhod_{—}773R: 5′-CAT TGA CAG GAC AGG AGA AGG GA-3′), 200 μM of each dNTP, ThermalAce™ buffer (Invitrogen Corp.), sterile water, and 2 units ThermalAce™ (add last). When present, SSB was included at concentrations of 0.1, 0.2 or 0.4 μg. Reactions were mixed thoroughly after adding ThermalAce™ and place on ice prior to thermocycling. Thermocycling parameters were as follows:

| Step | Temperature | Time | Cycle |
|---|---|---|---|
| Initial | 95° C. | 3 minutes | 1 |
| Denaturation | | | |
| Denaturation | 95° C. | 30 seconds | 25-30 |
| Annealing | 55° C. (5° C. < Tm) | 30 seconds | |
| Extension | 74° C. | 1 minute/kb | |
| Final Extension | 74° C. | 10 minutes | 1 |

After thermocycling, reactions were held at 4° C. or stored at a lower temperature. Reactions were electrophoresed on 0.8% horizontal agarose gel.

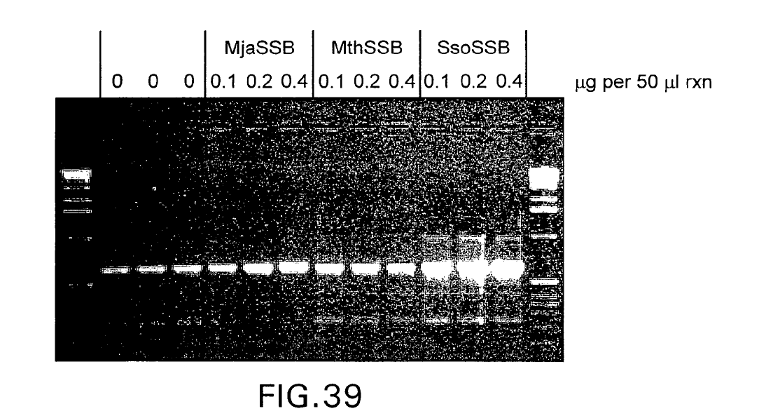

In general, SSBs increased the yield of the specific PCR product (FIG. 39). MjaSSB increased yield of the specific PCR product and decreased the yield of non-specific PCR products. MthSSB increased the yield of the specific PCR product to a level similar to that observed with MjaSSB but without decreasing the yield of non specific products. SsoSSB increased the yield of both the specific and non-specific PCR products.

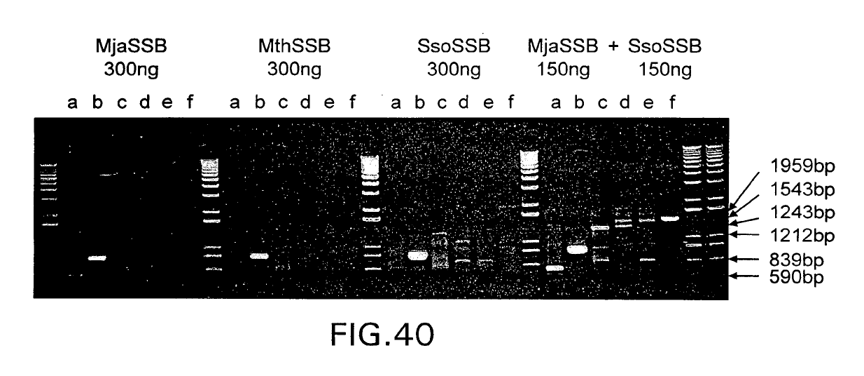

Another assay was performed using a fixed amount of SSB (either 300 ng of one SSB or 150 ng of each of two different SSBs in combination), and using six different primer sets targeting K562 amplicons ranging in size from 590 to 1959 bp. The primer sets were: a) p53 590 bp; b) p53 839 bp; c) p53 1212 bp; d) c-myc 1243 bp; e) c-myc 1543 bp; and f) c-myc 1959 bp. The combination of MjaSSB and SsoSSB was observed to increase the yield such that the combined effect of the SSBs appeared to be greater than the sum of the effects of the SSBs added individually (FIG. 40). SSBs in combination, surprisingly, appear to complement one another.

### Example 7

### Use of SSBS in Cycle Sequencing with Fluorescent Dye Terminators

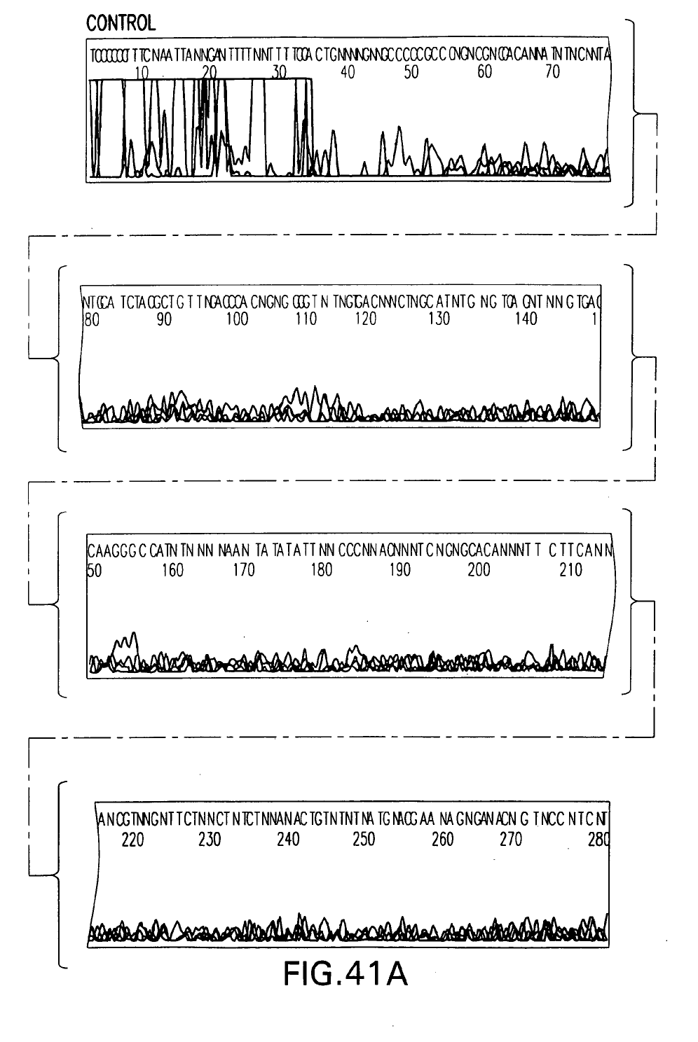

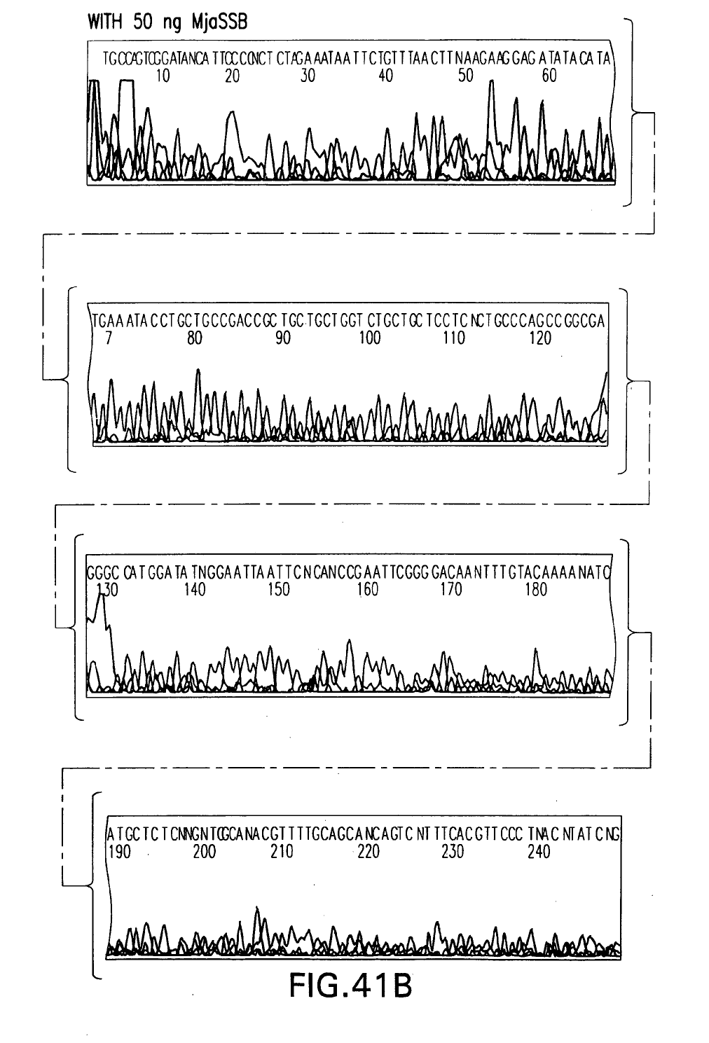

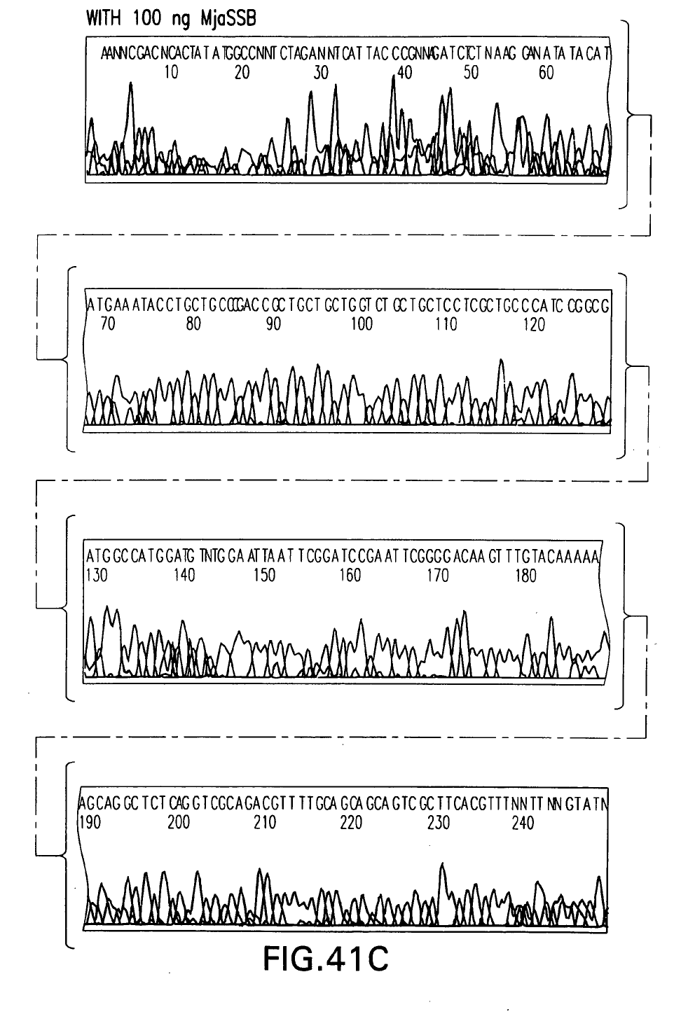

We tested whether Methanococcus jannachii SSB (MjaSSB) can improve cycle sequencing with fluorescent dye terminators. Cycle sequencing was done using an ABI Prism® 377 DNA Sequencer, ABI Prism® BigDye™ Terminator Cycle Sequencing Kits, 0.25× BigDye ReadiReaction Mix, and varying amounts of MjaSSB. Sequencing reactions included 500 ng of template (a plasmid having a gene cloned between attB sites) and 3.2 pmol of T7 promoter sequencing primer (5′-TAATACGACTCACTATAGGG-3′) per 20 μl reaction. MjaSSB was included at 50 or 100 ng per reaction. Panel A in FIG. 41 shows the result of a cycle sequencing reaction in the absence of SSB. The peak pileup (signal conflation) around position 35 and unreadable sequence thereafter may be caused by attB secondary structure. Addition of MjaSSB obviated the peak pileup and increased the length of readable sequence (Panels B and C in FIG. 41).

Other cycle sequencing reactions were performed as described above, but using different concentrations of MjaSSB (20 and 200 ng per 20 μl reaction), a pCMV-Sport6 expression construct as template, and 1.9 pmol of primer. About 90 sequencing reactions for each MjaSSB amount (i.e., 0, 20 and 200 ng) were analyzed using Phred (see e.g., Ewing, B. et al. (1998) Genome Res. 8:175-185 and Ewing, B. and P. Green (1998) Genome Res. 8:186-194). The number of called bases having a Q value greater than 20 (indicating a base calling error rate of less than 1%) were scored. When 20 ng MjaSSB was present in a sequencing reaction, the number of bases having a Q value greater than 20 was on average about 70%. Similarly, when 200 ng MjaSSB was present, the number of bases having Q values greater than 20 increased on average about 55%.

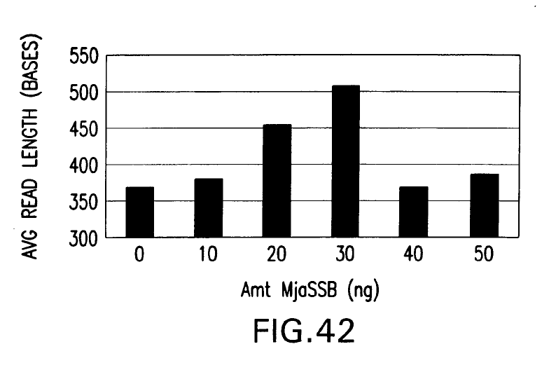

Other sequencing reactions were performed as described immediately above, except that the amount of MjaSSB was titrated to maximize the length of readable sequence. Readable sequence was defined as the number of bases in a sequencing reaction having Phred Q values greater than 20. FIG. 42 shows that adding 30 ng of MjaSSB increased readable sequence length from about 370 to over 500 bases.

### Example 8

### Codon Optimization to Enhance Expression of Archaeal SSB in a Bacterial Host

Introduction. Codon bias can be problematic when a eukaryotic or archaeal protein is cloned and expressed in bacteria, or vice versa. Problems related to codon bias include truncated peptide products, frame shift mutation, point mutation, and general inefficiency or inhibition of protein synthesis leading to arrested cell growth in extreme cases. Four methods are commonly employed to avoid problems associated with codon bias are: 1) co-expression of rare tRNAs (e.g., using commercially available strains complemented with the rare tRNA genes); 2) c-terminal affinity tagging so that only the full length polypeptide can be purified; 3) site-directed mutagenesis to replace rare codons with more common ones; and 4) using an alternative host having a more compatible codon usage. Using the third approach, one or more rare codons in a gene (e.g., a gene encoding an SSB) can be optimized to improve expression in a particular host, depending on the desired expression level. Thus, a single rare codon or a larger percentage (e.g., 10%, 20%, 30%, 40%, 50%, 60%, 70%, 80%, 90%, and 100%) of rare codons can be optimized in an SSB gene.

Attempts to express and purify archaeal SSBs in E. coli have met with problems associated with codon bias. For example, SSBs from Methanococcus jannachii (MjaSSB) and Sulfolobus solfataricus (SsoSSB) are expressed at relatively low levels in BL21(DE3) cells. In addition, SsoSSB co-purifies with shorter peptides most likely truncated proteins arising from premature termination. Present in high enough amounts, the shorter peptides can negate the SsoSSB-mediated PCR improvement.

The MjaSSB and SsoSSB genes use codons that are rarely used in E. coli. For example, in the native MjaSSB and SsoSSB genes AGA or AGG call for arginine, ATA calls for isoleucine, and CTA calls for leucine (Tables 16 and 17; rare codons are underlined). Many of these rare codons occur in tandem pairs or triplets, which may be responsible for the low expression level and/or truncated peptide contaminants.

TABLE 16
Sequence of the native SsoSSB gene

atg gaa gaa aaa gta ggt aat CTA aaa cca aat atg
gaa agc gta aat gta acc gta AGA gtt ttg gaa gca
agc gaa gca AGA caa ATA cag aca aag aac ggt gtt
AGA aca atc agt gag gct att gtt gga gat gaa acg
gga AGA gta aag tta aca tta tgg gga aaa cat gca
ggt agt ATA aaa gaa ggt caa gtg gta aag ATA gaa
aac gcg tgg acc acc gct ttt aag ggt caa gta cag
tta aat gct gga agc aaa act aag ATA gct gaa gct
tca gaa gat gga ttt cca gaa tca tct caa ATA cca
gaa aat aca cca aca gct cct cag caa atg cgt gga
gga gga AGA gga ttc cgc ggt ggg gga AGA AGG tat
gga AGA AGA ggt ggt AGA AGA caa gaa aac gaa gaa
ggt gaa gag gag tga

TABLE 17
Sequence of the native MjaSSB gene

atg gta gga gat tat gaa AGA ttt aaa caa ctc aaa
aaa aag gtt gct gaa gca ttg aat att aqt gag gag
gaa tta gat AGG atg att gat aaa aaa att gaa gaa
aac gga gga ATA ATA ttg aaa gat gct gca tta atg
atg att gca aaa gaa cat gga gtt tat gga gaa gaa
aaa aat gat gaa gaa ttt tta att agt gat att gaa
gag gga cag ATA ggc gtt gag ATA act gga gtt ATA
act gat atc tct gaa ATA aaa aca ttc aaa AGG AGA
gat ggg agt tta ggg aaa tac aaa AGA att aca ATA
gcg gat aag tca gga act ATA AGA atg act tta tgg
gac gat ttg gct gaa tta gat gta aaa gtt gga gat
gtt att aaa att gaa AGA gca AGA gca AGA aaa tgg
AGA aat aat tta gag ttg agt tca aca tct gaa act
aag att aaa aaa tta gaa aac tat gaa gga gaa ctt
cca gag att aaa gat acc tac aat att ggt gag CTA
agt cct gga atg aca gca aca ttt gaa gga gaa gtt
atc tca gct ctt cca atc aaa gaa ttt aaa AGA gct
gat ggt agt att gga aaa tta aaa tca ttt att gtt
AGA gat gag aca gga agt ATA AGA gtt acc tta tgg
gat aat CTA aca gat atc gat gtt ggt AGA gga gat
tac gtt AGA gtt AGG ggc tat ATA AGG gaa ggt tat
tat ggg ggt tta gaa tgc acc gca aat tat gta gag
ATA tta aaa aaa gga gaa aaa ATA gag agt gaa gaa
gta aat att gag gat tta aca aaa tat gaa gat gga
gaa ctg gtg agt gtt aaa ggt AGA gtt ATA gcc ATA
agt aat aaa aaa agc gta gat ttg gat gga gag ATA
gca aag gtt caa gat att ATA tta gat aac ggc act
ggt AGA gtt AGA gtt tca ttt tgg AGA gga aaa act
gct tta ttg gaa aat ATA aaa gaa ggg gac tta gtt
AGA ATA aca aac tgt AGA gtt aag acg ttt tat gat
AGA gaa gga aat aaa AGA act gat tta gtt gcc aca
tta gaa aca gaa gtt att aaa gat gaa aac att gaa
gct cca gag tat gag CTA aaa tat tgc aaa att gaa
gat att tat aat AGA gat gtt gac tgg aac gat ATA
aat tta ATA gct caa gtt gtt gag gat tat gga gtt
aat gaa att gaa ttt gaa gat aag gtt AGA aaa gta
AGA aat tta ttg tta gaa gat gga act gga AGA ATA
AGG ttg agt tta tgg gat gat ttg gct gaa ATA gag
att aaa gaa gga gat att gta gaa att tta cat gcc
tat gct aag gag AGG gga gat tat ATA gat ttg gtt
att gga aaa tat gga AGA ATA att ATA aat cca gaa
ggg gtt gaa ATA aaa acc aat AGA aag ttt ATA gca
gat att gaa gac gga gaa act gtt gaa gtt AGA ggg
gct gta gtt aag ATA ttg agt gac act ctc ttt ctt
tat tta tgc cca aat tgt AGA aag AGG gtt gta gag
att gat gga att tat aac tgc cct att tgt gga gat
gtt gag cca gaa gag att tta AGA ttg aat ttt gtt
gta gat gat ggg act gga act tta tta tgt AGG gct
tat gat AGA AGA gtt gag aag atg tta aaa atg aat
AGG gag gag tta aag aac CTA act ATA gaa atg gtg
gaa gat gaa ATA tta ggg gaa gag ttt gtt ttg tat
gga aat gtt AGA gta gag aat gat gaa tta att atg
gtt gtt AGA AGA gtt aat gat gta gat gtt gag aaa
gaa ATA AGA ATA ttg gag gaa atg gaa taa

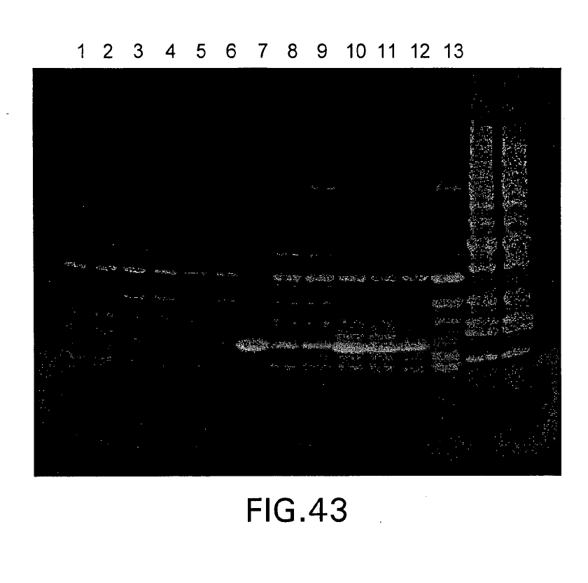

Codon optimization of SsoSSB. To test whether low expression of SsoSSB was related to codon bias, the native gene was transformed into BL21 CodonPlus with supplementary tRNA genes for Arg (AGA, AGG), Ile (AUA) and Leu (CUA) rare codons (Stratagene). When expressed in this host, a SsoSSB was produced at higher levels (compare lanes 12 and lane 13 in FIG. 43), and less truncated peptide was present after purification (compare FIGS. 30 & 31 with FIG. 28).

We replaced the rare codons in the SsoSSB gene with codons common in E. coli using “synthetic gene” technology (Stemmer, W. P. et al. (1995) Gene 164:49-53). Thus, AGA and AGG were replaced by CGG, CGT, CGA CGC; ATA was replaced by ATT or ATC; and CTA was replaced by CTT, CTG, or CTA (Table 18; optimized codons are underlined and in bold italics).

TABLE 18
Codon optimized recombinant SsoSSB gene

atg gaa gaa aaa gta ggt aat aaa cca aat atg
gaa agc gta aat gta acc gta cga gtt ttg gaa gca
agc gaa gca caa cag aca aag aac ggt gtt
aca atc agt gag gct att gtt gga gat gaa acg
gga gta aag tta aca tta tgg gga aaa cat gca
ggt agt aaa gaa ggt caa gtg gta aag gaa
aac gcg tgg acc acc gct ttt aag ggt caa gta cag
tta aat gct gga agc aaa act aag gct gaa gct
tca gaa gat gga ttt cca gaa tca tct caa cca
gaa aat aca cca aca gct cct cag caa atg cgt gga
gga gga gga ttc cgc ggt ggg gga tat
gga ggt ggt caa gaa aac gaa gaa
ggt gaa gag gag tga

To make a codon optimized SsoSSB gene, 21 overlapping primers were used (Table 19). The primers were mixed together in equal amounts (approximately 4.5 uM) in a PCR reaction without template DNA. PCR was performed using Taq Hi-FI Supermix (Invitrogen Corp.). A thermocycler was programed for 20 cycles of denaturation at 94° C. for 30 sec, annealing at 55° C. for 30 sec, and elongation at 72° C. for 30. An aliquot of this PCR reaction (2 μl or 1/25 of the volume) was added to a second PCR reaction with two 2 anchor primers that anneal at the 5′ and 3′ ends of the reassembled gene (Table 19). These primers also add a NdeI site to the 5′ end and a BamHI site to the 3′ end of the gene. After 2 more rounds of PCR using the parameters set out above, a discrete product of about 450 base pairs was obtained. The product was excised from an electrophoresis gel, purified, and cloned into pET21a vector at the NdeI and BamHI sites in the multi-cloning site. The resulting clone was sequenced to confirm the sequence.

TABLE 19
Forward Primers

| | |
|---|---|
| Sso F1 | ATGGAAGAAA AAGTAGGTAA TCTGAAACCA AATATGGAAA GC |
| Sso F2 | GTAAATGTAA CCGTACGAGT TTTGGAAGCA AGCGAAGCAC GT |
| Sso F3 | CAAATCCAGA CAAAGAACGG TGTTCGGACA ATCAGTGAGG CT |
| Sso F4 | ATTGTTGGAG ATGAAACGGG ACGAGTAAAG TTAACATTAT GG |
| Sso F5 | GGAAAACATG CAGGTAGTAT CAAAGAAGGT AAGTGGTAAA G |
| Sso F6 | ATTGAAAACG CGTGGACCAC CGCTTTTAAG GGTCAAGTAC AG |
| Sso F7 | TTAAATGCTG GAAGCAAAAC TAAGATCGCT GAAGCTTCAG AA |
| Sso F8 | GATGGATTTC CAGAATCATC TCAAATTCCA GAAAATACAC CA |
| Sso F9 | ACAGCTCCTC AGCAAATGCG TGGAGGAGGA CGCGGATTCC GC |
| Sso F10 | GGTGGGGGAC GTCGGTATGG ACGACGTGGT GGTCGCCGGC AA |
| Sso F11 | GAAAACGAAG AAGGTGAAGA GGAGTGA |

Reverse Primers

| | |
|---|---|
| Sso R1 | TCACTCCTCT TCACCTTCTT CGTTTTCTTG CCGGCGACCA CC |
| Sso R2 | ACGTCGTCCA TACCGACGTC CCCCACCGCG GAATCCGCGT CC |
| Sso R3 | TCCTCCACGC ATTTGCTGAG GAGCTGTTGG TGTATTTTCT GG |
| Sso R4 | AATTTGAGAT GATTCTGGAA ATCCATCTTC TGAAGCTTCA GC |
| Sso R5 | GATCTTAGTT TTGCTTCCAG CATTTAACTG TACTTGACCC TT |
| Sso R6 | AAAAGCGGTG GTCCACGCGT TTTCAATCTT TACCACTTGA CC |
| Sso R7 | TTCTTTGATA CTACCTGCAT GTTTTCCCCA TAATGTTAAC TT |
| Sso R8 | TACTCGTCCC GTTTCATCTC CAACAATAGC CTCACTGATT GT |
| Sso R9 | CCGAACACCG TTCTTTGTCT GGATTTGACG TGCTTCGCTT GC |
| Sso R10 | TTCCAAAACT CGTACGGTTA CATTTACGCT TTCCATATTT GG |
| Sso R11 | TTTCAGATTA CCTACTTTTT CTTCCAT |

Anchor primers

| | |
|---|---|
| Sso F NdeI | AATTCATATG GAAGAAAAAGT AGGT |
| Sso R BamHI | GGAAGGATCC TCACTCCTCTT CACCTTC |

A pET21a vector containing the recombinant codon optimized SsoSSB gene was transformed into BL21(DE3) and BL21(DE3)-AI (Arabinose Induced) strains. The level of SsoSSB present in lysates of induced and uninduced cultures was compared to the amount of SsoSSB obtained by expressing the native SsoSSB gene in BL21(DE3)-AI and BL21-CodonPlus. Cells were lysed by sonication, heated at 80° C. for 1 hour and the soluble fractions were run on an SDS gel along with purified protein as a marker (FIG. 43). Little if any expression was observed in uninduced cultures (FIG. 43, Lanes 1 to 6). When induced, rSsoSSB yielded equal or better expression in BL21(DE3) cells than native SsoSSB in BL21-CodonPlus (compare lanes 10 & 11 with lane 12 in FIG. 43). Grater expression was also observed using rSsoSSB in BL21(DE3)-AI, relative to native SsoSSB (compare lanes 8 & 9 with lane 13 in FIG. 43).

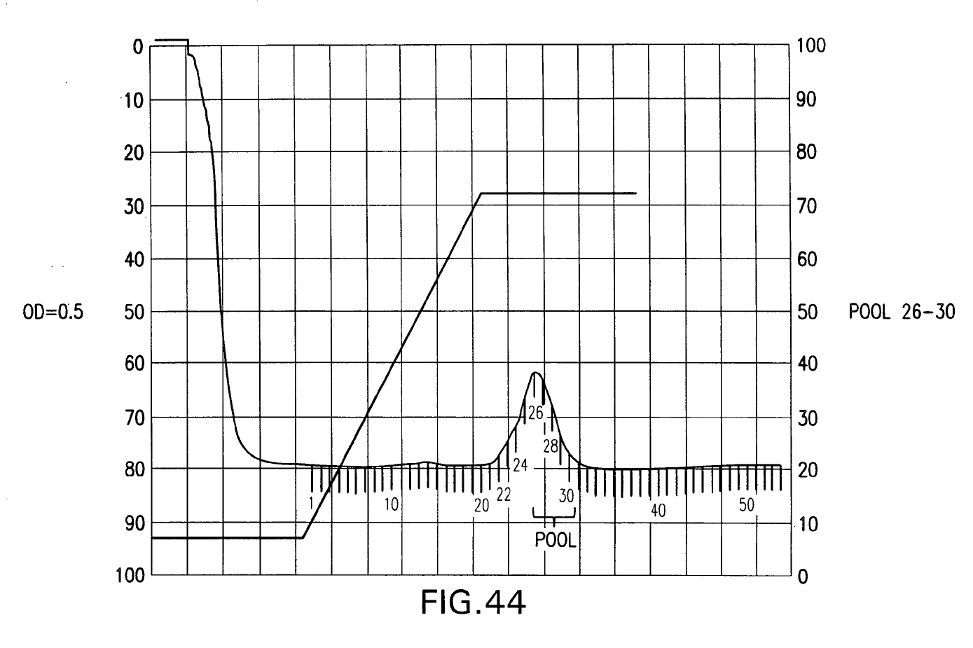

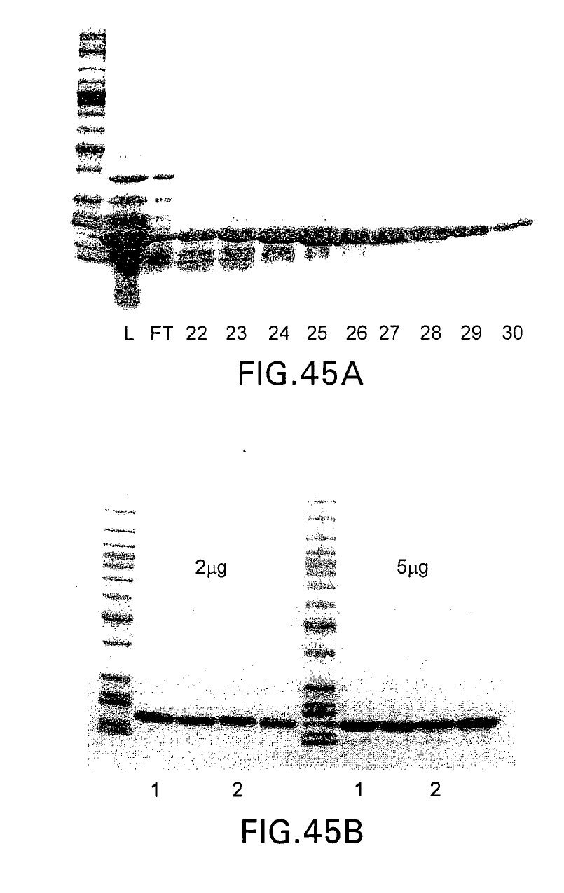

SsoSSB protein was purified from 2 liters of culture from BL21(DE3) hosts expressing rSsoSSB. Purification was done as described in Example 5, except that the culture was grown in LB media supplemented with ampicillin. Briefly, cells were grown in 2 liter LB media supplemented with ampicillin to an OD of 1.0 and protein expression was induced by adding IPTG to a final concentration of 1 mM. After 2 hours, cells were harvested by centrifugation, lysed by sonication, heat-treated at 80° C. for 1 hour, and clarified by centrifugation. The soluble fraction was loaded on a 10 ml EMD-SO₃ column, and was eluted first with a linear gradient of 50 to 650 mM NaCl and then with a 650 mM NaCl (FIG. 44). Fractions were analyzed by SDS PAGE and fractions containing 17.4 KDa protein were pooled (FIGS. 44 & 45A). The pool was dialyzed against storage buffer (20 mM NaCl, 20 mM Tris, pH 7.5, 1 mM EDTA, 1 mM DTT and 10% glycerol). The resultant protein preparation was observed by SDS-PAGE to be greater than 95% pure (FIG. 45B).

Codon optimization of MjaSSB. The sequence of a codon optimized MjaSSB gene is presented in Table 20, with optimized underlined and in bold italics).

TABLE 20
Codon optimized recombinant MjaSSB gene

```
atg gta gga gat tat gaa  ttt aaa caa ctc aaa
aaa aag gtt gct gaa gca ttg aat att agt gag gag
gaa tta gat 10 atg att gat aaa aaa att gaa gaa
aac gga gga  ttg aaa gat gct gca tta atg
atg att gca aaa gaa cat gga gtt tat gga gaa gaa
aaa aat gat gaa gaa ttt tta att agt gat att gaa
gag gga cag  ggc gtt gag  act gga gtt
act gat atc tct gaa  aaa aca ttc aaa
gat ggg agt tta ggg aaa tac aaa  att aca
gcg gat aag tca gga act  atg act tta tgg
gac gat ttg gct gaa tta gat gta aaa gtt gga gat
gtt att aaa att gaa  gca  gca  aaa tgg
  aat aat tta gag ttg agt tca aca tct gaa act
aag att aaa aaa tta gaa aac tat gaa gga gaa ctt
cca gag att aaa gat acc tac aat att ggt gag
agt cct gga atg aca gca aca ttt gaa gga gaa gtt
atc tca gct ctt cca atc aaa gaa ttt aaa gct
gat ggt agt att gga aaa tta aaa tca ttt att gtt
  gat gag aca gga agt  gtt acc tta tgg
gat aat  aca gat atc gat gtt ggt  gga gat
tac gtt  gtt  ggc tat  gaa ggt tat
tat ggg ggt tta gaa tgc acc gca aat tat gta gag
  tta aaa aaa gga gaa aaa  gag agt gaa gaa
gta aat att gag gat tta aca aaa tat gaa gat gga
gaa ctg gtg agt gtt aaa ggt  gtt  gcc
agt aat aaa aaa agc gta gat ttg gat gga gag
gca aag gtt caa gat att  tta gat aac ggc act
ggt  gtt  gtt tca ttt tgg CGG gga aaa act
gct tta ttg gaa aat  aaa gaa ggg gac tta gtt
  aca aac tgt  gtt aag acg ttt tat gat
  gaa gga aat aaa  act gat tta gtt gcc aca
tta gaa aca gaa gtt att aaa gat gaa aac att gaa
gct cca gag tat gag  aaa tat tgc aaa att gaa
gat att tat aat  gat gtt gac tgg aac gat
aat tta  gct caa gtt gtt gag gat tat gga gtt
aat gaa att gaa ttt gaa gat aag gtt  aaa gta
  aat tta ttg tta gaa gat gga act gga
  ttg agt tta tgg gat gat ttg gct gaa  gag
att aaa gaa gga gat att gta gaa att tta cat gcc
tat gct aag gag  gga gat tat  gat ttg gtt
att gga aaa tat gga    att  aat cca gaa
ggg gtt gaa  aaa acc aat  aag ttt  gca
gat att gaa gac gga gaa act gtt gaa gtt  ggg
gct gta gtt aag  ttg agt gac act ctc ttt ctt
tat tta tgc cca aat tgt  aag  gtt gta gag
att gat gga att tat aac tgc cct att tgt gga gat
gtt gag cca gaa gag att tta  ttg aat ttt gtt
gta gat gat ggg act gga act tta tta tgt  gct
tat gat  gtt gag aag atg tta aaa atg aat
  gag gag tta aag aac  act  gaa atg gtg
gaa gat gaa  tta ggg gaa gag ttt gtt ttg tat
gga aat gtt  gta gag aat gat gaa tta att atg
gtt gtt  gtt aat gat gta gat gtt gag aaa
gaa  ttg gag gaa atg gaa taa
```

The primers identified in Table 21 are used to replace the rare codons in the MjaSSB gene with codons common in E. coli using “synthetic gene” technology, as was done for the SsoSSB gene. The forward and reverse primers are about 60 nucleotide long and overlapping at least 15 nucleotides with the neighboring primers.

TABLE 21
Forward Primers

| | |
|---|---|
| Mja F1 | ATGGTAGGAG ATTATGAACG TTTTAAACAA CTCAAAAAAA AGGTTGCTGA AGCATTGAAT |
| Mja F2 | GATAAAAAAA TTGAAGAAAA CGGAGGAATC ATTTTGAAAG ATGCTGCATT AATGATGATT |
| Mja F3 | AAAAATGATG AAGAATTTTT AATTAGTGAT ATTGAAGAGG GACAGATTGG CGTTGAGATC |
| Mja F4 | AAAACATTCA AACGGCGCGA TGGGAGTTTA GGGAAATACA AACGAATTAC AATTGCGGAT |
| Mja F5 | GACGATTTGG CTGAATTAGA TGTAAAAGTT GGAGATGTTA TTAAAATTGA ACGCGCACGG |
| Mja F6 | AGTTCAACAT CTGAAACTAA GATTAAAAAA TTAGAAAACT ATGAAGGAGA ACTTCCAGAG |
| Mja F7 | AGTCCTGGAA TGACAGCAAC ATTTGAAGGA GAAGTTATCT CAGCTCTTCC AATCAAAGAA |
| Mja F8 | TTAAAATCAT TTATTGTTCG CGATGAGACA GGAAGTATTC GCGTTACCTT ATGGGATAAT |
| Mja F9 | TACGTTCGTG TTCGGGGCTA TATCCGGGAA GGTTATTATG GGGGTTTAGA ATGCACCGCA |
| Mja F10 | AAAATAGAGA GTGAAGAAGT AAATATTGAG GATTTAACAA AATATGAAGA TGGAGAACTG |
| Mja F11 | AGTAATAAAA AAAGCGTAGA TTTGGATGGA GAGATTGCAA AGGTTCAAGA TATTATCTTA |
| Mja F12 | TTTTGGCGGG GAAAAACTGC TTTATTGGAA AATATCAAAG AAGGGGACTT AGTTCGTATC |
| Mja F13 | CGTGAAGGAA ATAAACGGAC TGATTTAGTT GCCACATTAG AAACAGAAGT TATTAAAGAT |
| Mja F14 | AAATATTGCA AAATTGAAGA TATTTATAAT CGCGATGTTG ACTGGAACGA TATAAATTTA |
| Mja F15 | AATGAAATTG AATTTGAAGA TAAGGTTCGT AAAGTACGCA ATTTATTGTT AGAAGATGGA |
| Mja F16 | GATTTGGCTG AAATTGAGAT TAAAGAAGGA GATATTGTAG AAATTTTACA TGCCTATGCT |
| Mja F17 | ATTGGAAAAT ATGGACGAAT TATTATCAAT CCAGAAGGGG TTGAAATCAA AACCAATCGT |
| Mja F18 | ACTGTTGAAG TTCGCGGGGC TGTAGTTAAG ATCTTGAGTG ACACTCTCTT TCTTTATTTA |
| Mja F19 | ATTGATGGAA TTTATAACTG CCCTATTTGT GGAGATGTTG AGCCAGAAGA GATTTTACGA |
| Mja F20 | ACTTTATTAT GTCGGGCTTA TGATCGCCGT GTTGAGAAGA TGTTAAAAAT GAATCGGGAG |
| Mja F21 | GAAGATGAAA TTTTAGGGGA AGAGTTTGTT TTGTATGGAA ATGTTCGAGT AGAGAATGAT |
| Mja F22 | GATGTAGATG TTGAGAAAGA AATTCGTATC TTGGAGGAAA TGGAATAA |

Reverse Primers

| | |
|---|---|
| Mja R1 | TTCAATTTTT TTATCAATCA TCCGATCTAA TTCCTCCTCA CTAATATTCA ATGCTTCAGC |
| Mja R2 | TTCTTCATCA TTTTTTTCTT CTCCATAAAC TCCATGTTCT TTTGCAATCA TCATTAATGC |
| Mja R3 | CCGTTTGAAT GTTTTGATTT CAGAGATATC AGTAATAACT CCAGTGATCT CAACGCCAAT |
| Mja R4 | TTCAGCCAAA TCGTCCCATA AAGTCATACG AATAGTTCCT GACTTATCCG CAATTGTAAT |
| Mja R5 | TTCAGATGTT GAACTCAACT CTAAATTATT TCGCCATTTA CGTGCCCGTG CGCGTTCAAT |
| Mja R6 | TGTCATTCCA GGACTCAGCT CACCAATATT GTAGGTATCT TTAATCTCTG GAAGTTCTCC |
| Mja R7 | AATAAATGAT TTTAATTTTC CAATACTACC ATCAGCACGT TTAAATTCTT TGATTGGAAG |
| Mja R8 | CCGAACACGA ACGTAATCTC CACGACCAAC ATCGATATCT GTAAGATTAT CCCATAAGGT |
| Mja R9 | TTCACTCTCT ATTTTTTCTC CTTTTTTTAA AATCTCTACA TAATTTGCGG TGCATTCTAA |
| Mja R10 | GCTTTTTTTA TTACTGATGG CAATAACTCG ACCTTTAACA CTCACCAGTT CTCCATCTTC |
| Mja R11 | TTTTCCCCGC CAAAATGAAA CACGAACTCG ACCAGTGCCG TTATCTAAGA TAATATCTTG |
| Mja R12 | TTTATTTCCT TCACGATCAT AAAACGTCTT AACGCGACAG TTTGTGATAC GAACTAAGTC |
| Mja R13 | AATTTTGCAA TATTTCAGCT CATACTCTGG AGCTTCAATG TTTTCATCTT TAATAACTTC |
| Mja R14 | AAATTCAATT TCATTAACTC CATAATCCTC AACAACTTGA GCGATTAAAT TTATATCGTT |
| Mja R15 | AATTTCAGCC AAATCATCCC ATAAACTCAA CCGAATACGT CCAGTTCCAT CTTCTAACAA |
| Mja R16 | TCCATATTTT CCAATAACCA AATCGATATA ATCTCCCCGC TCCTTAGCAT AGGCATGTAA |
| Mja R17 | GCGAACTTCA ACAGTTTCTC CGTCTTCAAT ATCTGCAATA AACTTACGAT TGGTTTTGAT |
| Mja R18 | ATAAATTCCA TCAATCTCTA CAACCCGCTT ACGACAATTT GGGCATAAAT AAAGAAAGAG |
| Mja R19 | CCGACATAAT AAAGTTCCAG TCCCATCATC TACAACAAAA TTCAATCGTA AAATCTCTTC |
| Mja R20 | TAAAATTTCA TCTTCCACCA TTTCGATAGT AAGGTTCTTT AACTCCTCCC GATTCATTTT |
| Mja R21 | CTCAACATCT ACATCATTAA CGCGACGAAC AACCATAATT AATTCATCAT TCTCTACTCG |
| Mja R22 | TTATTCCATT TCCTCCAAGA TACGAATTTC TTT |

Anchor primers

| | |
|---|---|
| Mja F | GCTGCCATGG TAGGAGATTA TGAACGTTTT AAACAAC |
| Mja R | GCTCCTCGAG TTATTCCATT TCCTCCAAGA TACG |

The present invention has been described with reference to certain embodiments thereof. However, the scope of the invention is not limited to the embodiments described. Workers of ordinary skill in the art will readily appreciate that other embodiments can be practiced without departing from the scope of the present invention. All such variations are considered to be part of, and therefore encompassed by, this invention.

All publications, patents and patent applications mentioned in this specification are indicative of the level of skill of those skilled in the art to which the present invention pertains, and are herein incorporated by reference to the same extent as if each individual publication, patent or patent application was specifically and individually indicated to be incorporated by reference.

What is claimed is:

1. A composition comprising:
(a) at least one anti-DNAP antibody and/or at least one anti-RT antibody; and
(b) at least one SSB.

2. A composition comprising at least two different SSBs.

3. The composition of claim 1 or claim 2, further comprising one or more nucleic acid templates.

4. The composition of claim 3, wherein at least one of said one or more templates is a cDNA.

5. The composition of claim 1 or claim 2, further comprising at least one primer.

6. The composition of claim 1 or claim 2, further comprising one or more nucleotides.

7. The composition of claim 1 or claim 2, further comprising one or more DNAPs.

8. The composition of claim 1 or claim 2, further comprising one or more reverse transcriptases.

9. A method for synthesizing a nucleic acid, said method comprising:
(a) providing a mixture comprising one or more nucleic acid templates, one or anti-DNAP antibodies and/or one or more anti-RT antibodies, and one or more SSBs; and
(b) incubating said mixture under conditions sufficient to synthesize a nucleic acid molecule complementary to all or a portion of said one or more templates.

10. The method of claim 9, wherein said step (a) is accomplished at a temperature sufficient to inhibit nucleic acid synthesis, and wherein said step (b) is accomplished at a temperature sufficient to reduce the inhibitory affect of said one or more antibodies.

11. A method for synthesizing a nucleic acid, said method comprising:
(a) providing a mixture comprising one or more nucleic acid templates and two or more SSBs; and
(b) incubating said mixture under conditions sufficient to make a nucleic acid complementary to all or a portion of said one or more templates.

12. The method of claim 9 or claim 11, wherein at least one of said one or more templates is cDNA.

13. The method of claim 9 or claim 11, wherein at lest one of said one or more templates is RNA.

14. The method of claim 9 or claim 10, wherein said mixture further comprises at least one component selected from the group consisting of one or more primers, one or more nucleotides, one or more DNAPs, one or more reverse transcriptases, and one or more buffers or buffering salts.


SEQUENCE LISTING (164 sequences)

SEQ ID NO: 1 - DNA, 36 bp, Unknown
  Oligonucleotide
gggagacggggaattcgtcgacgcgtcaggactcta

SEQ ID NO: 2 - DNA, 84 bp, Unknown
  KP_PALIN_cont
ctcctggatcgacttcagtccgctgatgattagatgtcgtcctggatcgacttcactccgcacccgctaccaacaacagtaccc

SEQ ID NO: 3 - DNA, 30 bp, Unknown
  M13mp19_1442L30
gccgacaatgacaacaaccatcgcccacgc

SEQ ID NO: 4 - DNA, 21 bp, Unknown
  Rhod_147F
aggagcttaggagggggaggt

SEQ ID NO: 5 - DNA, 23 bp, Unknown
  Rhod_773R
cattgacaggacaggagaaggga

SEQ ID NO: 6 - DNA, 25 bp, Unknown
  pUC19_2182F
tcaaccaattcatcctgagaatagt

SEQ ID NO: 7 - DNA, 26 bp, Unknown
  puc19_2177r
tcaccagtcacagaaaagcatcttac

SEQ ID NO: 8 - DNA, 23 bp, Unknown
  LTI, 18431-015
ccgagtcacgacgttgtaaaacg

SEQ ID NO: 9 - DNA, 23 bp, Unknown
  LTI, 18432-013
agcggataacaatttcacacagg

SEQ ID NO: 10 - DNA, 20 bp, Unknown
  pUC19_606f
ccagtcgggaaacctgtcgt

SEQ ID NO: 11 - DNA, 20 bp, Unknown
  pUC19_745r
accgcctttgagtgagctga

SEQ ID NO: 12 - DNA, 18 bp, Unknown
  p32D9 149 bp, forward primer
atcccccacccccgcacc

SEQ ID NO: 13 - DNA, 18 bp, Unknown
  p32D9 149 bp, reverse primer
gggcgcgagatgggctgc

SEQ ID NO: 14 - DNA, 22 bp, Unknown
  Pr1.2 235 bp, forward primer
ttggaggggtgggtgagtcaag

SEQ ID NO: 15 - DNA, 23 bp, Unknown
  Pr1.2 235 bp, reverse primer
ggaggggtgggggttaatggtta

SEQ ID NO: 16 - DNA, 22 bp, Unknown
  Pr1.3 265 bp, forward primer
gcatctggggcctgggatttag

SEQ ID NO: 17 - DNA, 25 bp, Unknown
  Pr1.3 265 bp, reverse primer
tacaaggcaggcatcatgactcacg

SEQ ID NO: 18 - DNA, 22 bp, Unknown
  p53 gene 504 bp, forward primer
tgccgtcccaagcaatggattt

SEQ ID NO: 19 - DNA, 24 bp, Unknown
  p53 gene 504 bp, reverse primer
caggagagatgctgagggtgtgga

SEQ ID NO: 20 - DNA, 22 bp, Unknown
  c-myc gene 822 bp, forward primer
cggtccacaagctctccacttg

SEQ ID NO: 21 - DNA, 23 bp, Unknown
  c-myc gene 822 bp, reverse primer
ctgtttgacaaaccgcatccttg

SEQ ID NO: 22 - DNA, 24 bp, Unknown
  c-myc gene 1069 bp, forward primer
ggttttcggggctttatctaactc

SEQ ID NO: 23 - DNA, 21 bp, Unknown
  c-myc gene 1069 bp, reverse primer
gcctacccaacaccacgtcct

SEQ ID NO: 24 - DNA, 25 bp, Unknown
  p53 gene 1587 bp, forward primer
gctgccgtgttccagttgctttatc

SEQ ID NO: 25 - DNA, 21 bp, Unknown
  p53 gene 1587 bp, reverse primer
gcagctcgtggtgaggctccc

SEQ ID NO: 26 - DNA, 27 bp, Unknown
  p53 gene 1996 bp, forward primer
ccttggcttttgaaaataagctcctga

SEQ ID NO: 27 - DNA, 21 bp, p53 gene 1996 bp, reverse primer
gcagctcgtggtgaggctccc

SEQ ID NO: 28 - DNA, 24 bp, Unknown
  p53 gene 2108  bp, Forward primer
gcagagacctgtgggaagcgaaaa

SEQ ID NO: 29 - DNA, 22 bp, Unknown
  p53 gene 2108  bp, reverse primer
gagagctgtggcaagcagggga

SEQ ID NO: 30 - DNA, 24 bp, Unknown
  Rhodopsin gene 3047 bp, forward primer
gccctaacttctacgtgcccttct

SEQ ID NO: 31 - DNA, 21 bp, Unknown
  Rhodopsin gene 3047 bp, reverse primer
aggcttccagcgcacgtcatt

SEQ ID NO: 32 - DNA, 20 bp, Unknown
  p53 gene 4356 bp, forward primer
cccctcctggcccctgtcat

SEQ ID NO: 33 - DNA, 27 bp, Unknown
  p53 gene 4356 bp, reverse primer
gttagatgactttgcccaactgtaggg

SEQ ID NO: 34 - DNA, 22 bp, Unknown
  Tms1-44, forward primer
ggctggagtgcagtggtgcaat

SEQ ID NO: 35 - DNA, 22 bp, Unknown
  Tms1-44, reverse primer
ggcagaggctacagtgagccaa

SEQ ID NO: 36 - DNA, 24 bp, Unknown
  Thal-57, forward primer
gggcagagccatctattgcttaca

SEQ ID NO: 37 - DNA, 26 bp, Unknown
  Thal-57, reverse primer
ggttgctagtgaacacagttgtgtca

SEQ ID NO: 38 - DNA, 22 bp, Unknown
  Hba2-67, forward primer
gcactcttctggtccccacaga

SEQ ID NO: 39 - DNA, 22 bp, Unknown
  Hba2-67, reverse primer
ttggtcttgtcggcaggagaca

SEQ ID NO: 40 - DNA, 22 bp, Unknown
  Rgr-74, forward primer
cccacgatcaatgccatcaact

SEQ ID NO: 41 - DNA, 22 bp, Unknown
  Rgr-74, reverse primer
cggtgagaggcactgccagatt

SEQ ID NO: 42 - DNA, 22 bp, Unknown
  B-glo-thal-84, forward primer
gctcgctttcttgctgtccaat

SEQ ID NO: 43 - DNA, 24 bp, Unknown
  B-glo-thal-84, reverse primer
gcccttcataatatcccccagttt

SEQ ID NO: 44 - DNA, 20 bp, Unknown
  c-myc-100, forward primer
gtccttcccccgctggaaac

SEQ ID NO: 45 - DNA, 20 bp, Unknown
  c-myc-100, reverse primer
gcagcagagatcatcgcgcc

SEQ ID NO: 46 - DNA, 21 bp, Unknown
  Zip-116, forward primer
gtgggggtgctgggagtttgt

SEQ ID NO: 47 - DNA, 23 bp, Unknown
  Zip-116, reverse primer
tcggacagaaacatgggtctgaa

SEQ ID NO: 48 - DNA, 22 bp, Unknown
  Csh1-135, forward primer
ggtgctcagaacccccacaatc

SEQ ID NO: 49 - DNA, 22 bp, Unknown
  Csh1-135, reverse primer
cctaccgaccccattccactct

SEQ ID NO: 50 - DNA, 23 bp, Unknown
  Sub-153, forward primer
cacagatttccaaggatgcgctg

SEQ ID NO: 51 - DNA, 20 bp, Unknown
  Sub-153, reverse primer
cgtgctctgttccagacttg

SEQ ID NO: 52 - DNA, 22 bp, Unknown
  Svmt-170, forward primer
cgtctggcgattgctccaaatg

SEQ ID NO: 53 - DNA, 22 bp, Unknown
  Svmt-170, reverse primer
gggcagttgtgatccatgagaa

SEQ ID NO: 54 - DNA, 22 bp, Unknown
  Olf-183, forward primer
ggcttgcaccagcttaggaaag

SEQ ID NO: 55 - DNA, 25 bp, Unknown
  Olf-183, reverse primer
cgttaggcataatcagtgggatagt

SEQ ID NO: 56 - DNA, 26 bp, Unknown
  P53-193, forward primer
gcctctgattcctcactgattgctct

SEQ ID NO: 57 - DNA, 23 bp, Unknown
  P53-193, reverse primer
tgtcaaccacccttaacccctcc

SEQ ID NO: 58 - DNA, 22 bp, Unknown
  Pr 1.2-237, forward primer
ttggaggggtgggtgagtcaag

SEQ ID NO: 59 - DNA, 23 bp, Unknown
  Pr 1.2-237, reverse primer
ggaggggtgggggttaatggtta

SEQ ID NO: 60 - DNA, 21 bp, Unknown
  Hmk-243, forward primer
ggaacaagacacggctgggtt

SEQ ID NO: 61 - DNA, 19 bp, Unknown
  Hmk-243, reverse primer
agcaaggcagggcaggcaa

SEQ ID NO: 62 - DNA, 22 bp, Unknown
  Rhod-273, forward primer
cggtcccattctcagggaatct

SEQ ID NO: 63 - DNA, 22 bp, Unknown
  Rhod-273, reverse primer
gcccagaggaagaagaaggaaa

SEQ ID NO: 64 - DNA, 23 bp, Unknown
  Caaf1-300, forward primer
gcccccacccaggttggtttcta

SEQ ID NO: 65 - DNA, 22 bp, Unknown
  Caaf1-300, reverse primer
atgccttcatctggctcagtga

SEQ ID NO: 66 - DNA, 22 bp, Unknown
  P-450 B-319, forward primer
gctcagcatggtggtggcataa

SEQ ID NO: 67 - DNA, 21 bp, Unknown
  P-450 B-319, reverse primer
cctcataccttcccccccatt

SEQ ID NO: 68 - DNA, 25 bp, Unknown
  S-100-360, forward primer
gactactctagcgactgtccatctc

SEQ ID NO: 69 - DNA, 20 bp, Unknown
  S-100-360, reverse primer
gacagccaccagatccaatc

SEQ ID NO: 70 - DNA, 21 bp, Unknown
  B-cone-432, forward primer
ggcagctttcatgggcactgt

SEQ ID NO: 71 - DNA, 22 bp, Unknown
  B-cone-432, reverse primer
gacagggctggactgacatttg

SEQ ID NO: 72 - DNA, 21 bp, Unknown
  Hbg-469, forward primer
ctgctgaaagagatgcggtgg

SEQ ID NO: 73 - DNA, 22 bp, Unknown
  Hbg-469, reverse primer
aggaaaacagcccaagggacag

SEQ ID NO: 74 - DNA, 23 bp, Unknown
  priA_260_F
acgcgccgatgtggtactggttt

SEQ ID NO: 75 - DNA, 23 bp, Unknown
  priA_260_R
gcggtggcctgttcggtattcaa

SEQ ID NO: 76 - DNA, 20 bp, Unknown
  p53_2380_F
cccctcctggcccctgtcat

SEQ ID NO: 77 - DNA, 21 bp, Unknown
  p53_2380_R
gcagctcgtggtgaggctccc

SEQ ID NO: 78 - DNA, 24 bp, Unknown
  Hbg_3.6_F
ttcctgagagccgaactgtagtga

SEQ ID NO: 79 - DNA, 30 bp, Unknown
  Hbg_3.6_R
taagacatgtatttgcatggaaaacaactc

SEQ ID NO: 80 - DNA, 23 bp, Unknown
  Rhod_575_F
ccctctacacctctctgcatgga

SEQ ID NO: 81 - DNA, 23 bp, Unknown
  Rhod_6748_R
agcaacaaaacccaccaccgtta

SEQ ID NO: 82 - DNA, 23 bp, Unknown
  Rhod_532_F
gccgtggctgacctcttcatggt

SEQ ID NO: 83 - DNA, 20 bp, Unknown
  T7 promoter sequencing primer
taatacgactcactataggg

SEQ ID NO: 84 - DNA, 447 bp, Sulfolobus solfataricus
  Sso SSB gene
atggaagaaaaagtaggtaatctaaaaccaaatatggaaagcgtaaatgtaaccgtaagagttttggaagcaagcgaagcaagacaaatacagacaaagaacggtgttagaacaatcagtgaggctattgttggagatgaaacgggaagagtaaagttaacattatggggaaaacatgcaggtagtataaaagaaggtcaagtggtaaagatagaaaacgcgtggaccaccgcttttaagggtcaagtacagttaaatgctggaagcaaaactaagatagctgaagcttcagaagatggatttccagaatcatctcaaataccagaaaatacaccaacagctcctcagcaaatgcgtggaggaggaagaggattccgcggtgggggaagaaggtatggaagaagaggtggtagaagacaagaaaacgaagaaggtgaagaggagtga

SEQ ID NO: 85 - DNA, 1938 bp, Methanococcus jannachii
  Mja SSB gene
atggtaggagattatgaaagatttaaacaactcaaaaaaaaggttgctgaagcattgaatattagtgaggaggaattagataggatgattgataaaaaaattgaagaaaacggaggaataatattgaaagatgctgcattaatgatgattgcaaaagaacatggagtttatggagaagaaaaaaatgatgaagaatttttaattagtgatattgaagagggacagataggcgttgagataactggagttataactgatatctctgaaataaaaacattcaaaaggagagatgggagtttagggaaatacaaaagaattacaatagcggataagtcaggaactataagaatgactttatgggacgatttggctgaattagatgtaaaagttggagatgttattaaaattgaaagagcaagagcaagaaaatggagaaataatttagagttgagttcaacatctgaaactaagattaaaaaattagaaaactatgaaggagaacttccagagattaaagatacctacaatattggtgagctaagtcctggaatgacagcaacatttgaaggagaagttatctcagctcttccaatcaaagaatttaaaagagctgatggtagtattggaaaattaaaatcatttattgttagagatgagacaggaagtataagagttaccttatgggataatctaacagatatcgatgttggtagaggagattacgttagagttaggggctatataagggaaggttattatgggggtttagaatgcaccgcaaattatgtagagatattaaaaaaaggagaaaaaatagagagtgaagaagtaaatattgaggatttaacaaaatatgaagatggagaactggtgagtgttaaaggtagagttatagccataagtaataaaaaaagcgtagatttggatggagagatagcaaaggttcaagatattatattagataacggcactggtagagttagagtttcattttggagaggaaaaactgctttattggaaaatataaaagaaggggacttagttagaataacaaactgtagagttaagacgttttatgatagagaaggaaataaaagaactgatttagttgccacattagaaacagaagttattaaagatgaaaacattgaagctccagagtatgagctaaaatattgcaaaattgaagatatttataatagagatgttgactggaacgatataaatttaatagctcaagttgttgaggattatggagttaatgaaattgaatttgaagataaggttagaaaagtaagaaatttattgttagaagatggaactggaagaataaggttgagtttatgggatgatttggctgaaatagagattaaagaaggagatattgtagaaattttacatgcctatgctaaggagaggggagattatatagatttggttattggaaaatatggaagaataattataaatccagaaggggttgaaataaaaaccaatagaaagtttatagcagatattgaagacggagaaactgttgaagttagaggggctgtagttaagatattgagtgacactctctttctttatttatgcccaaattgtagaaagagggttgtagagattgatggaatttataactgccctatttgtggagatgttgagccagaagagattttaagattgaattttgttgtagatgatgggactggaactttattatgtagggcttatgatagaagagttgagaagatgttaaaaatgaatagggaggagttaaagaacctaactatagaaatggtggaagatgaaatattaggggaagagtttgttttgtatggaaatgttagagtagagaatgatgaattaattatggttgttagaagagttaatgatgtagatgttgagaaagaaataagaatattggaggaaatggaataa

SEQ ID NO: 86 - DNA, 447 bp, Unknown
  Codon optimized recombinant SsoSSB gene
atggaagaaaaagtaggtaatctgaaaccaaatatggaaagcgtaaatgtaaccgtacgagttttggaagcaagcgaagcacgtcaaatccagacaaagaacggtgttcggacaatcagtgaggctattgttggagatgaaacgggacgagtaaagttaacattatggggaaaacatgcaggtagtatcaaagaaggtcaagtggtaaagattgaaaacgcgtggaccaccgcttttaagggtcaagtacagttaaatgctggaagcaaaactaagatcgctgaagcttcagaagatggatttccagaatcatctcaaattccagaaaatacaccaacagctcctcagcaaatgcgtggaggaggacgcggattccgcggtgggggacgtcggtatggacgacgtggtggtcgccggcaagaaaacgaagaaggtgaagaggagtga

SEQ ID NO: 87 - DNA, 42 bp, Unknown
  Sso F1
atggaagaaaaagtaggtaatctgaaaccaaatatggaaagc

SEQ ID NO: 88 - DNA, 42 bp, Unknown
  Sso F2
gtaaatgtaaccgtacgagttttggaagcaagcgaagcacgt

SEQ ID NO: 89 - DNA, 42 bp, Unknown
  Sso F3
caaatccagacaaagaacggtgttcggacaatcagtgaggct

SEQ ID NO: 90 - DNA, 42 bp, Unknown
  Sso F4
attgttggagatgaaacgggacgagtaaagttaacattatgg

SEQ ID NO: 91 - DNA, 41 bp, Unknown
  Sso F5
ggaaaacatgcaggtagtatcaaagaaggtaagtggtaaag

SEQ ID NO: 92 - DNA, 42 bp, Unknown
  Sso F6
attgaaaacgcgtggaccaccgcttttaagggtcaagtacag

SEQ ID NO: 93 - DNA, 42 bp, Unknown
  Sso F7
ttaaatgctggaagcaaaactaagatcgctgaagcttcagaa

SEQ ID NO: 94 - DNA, 42 bp, Unknown
  Sso F8
gatggatttccagaatcatctcaaattccagaaaatacacca

SEQ ID NO: 95 - DNA, 42 bp, Unknown
  Sso F9
acagctcctcagcaaatgcgtggaggaggacgcggattccgc

SEQ ID NO: 96 - DNA, 42 bp, Unknown
  Sso F10
ggtgggggacgtcggtatggacgacgtggtggtcgccggcaa

SEQ ID NO: 97 - DNA, 27 bp, Unknown
  Sso F11
gaaaacgaagaaggtgaagaggagtga

SEQ ID NO: 98 - DNA, 42 bp, Unknown
  Sso R1
tcactcctcttcaccttcttcgttttcttgccggcgaccacc

SEQ ID NO: 99 - DNA, 42 bp, Unknown
  Sso R2
acgtcgtccataccgacgtcccccaccgcggaatccgcgtcc

SEQ ID NO: 100 - DNA, 42 bp, Unknown
  Sso R3
tcctccacgcatttgctgaggagctgttggtgtattttctgg

SEQ ID NO: 101 - DNA, 42 bp, Unknown
  Sso R4
aatttgagatgattctggaaatccatcttctgaagcttcagc

SEQ ID NO: 102 - DNA, 42 bp, Unknown
  Sso R5
gatcttagttttgcttccagcatttaactgtacttgaccctt

SEQ ID NO: 103 - DNA, 42 bp, Unknown
  Sso R6
aaaagcggtggtccacgcgttttcaatctttaccacttgacc

SEQ ID NO: 104 - DNA, 42 bp, Unknown
  Sso R7
ttctttgatactacctgcatgttttccccataatgttaactt

SEQ ID NO: 105 - DNA, 42 bp, Unknown
  Sso R8
tactcgtcccgtttcatctccaacaatagcctcactgattgt

SEQ ID NO: 106 - DNA, 42 bp, Unknown
  Sso R9
ccgaacaccgttctttgtctggatttgacgtgcttcgcttgc

SEQ ID NO: 107 - DNA, 42 bp, Unknown
  Sso R10
ttccaaaactcgtacggttacatttacgctttccatatttgg

SEQ ID NO: 108 - DNA, 27 bp, Unknown
  Sso R11
tttcagattacctactttttcttccat

SEQ ID NO: 109 - DNA, 25 bp, Unknown
  Sso F NdeI
aattcatatggaagaaaaagtaggt

SEQ ID NO: 110 - DNA, 28 bp, Unknown
  Sso R BamHI
ggaaggatcctcactcctcttcaccttc

SEQ ID NO: 111 - DNA, 1938 bp, Unknown
  Codon optimized recombinant MjaSSB gene
atggtaggagattatgaacgttttaaacaactcaaaaaaaaggttgctgaagcattgaatattagtgaggaggaattagatcggatgattgataaaaaaattgaagaaaacggaggaatcattttgaaagatgctgcattaatgatgattgcaaaagaacatggagtttatggagaagaaaaaaatgatgaagaatttttaattagtgatattgaagagggacagattggcgttgagatcactggagttattactgatatctctgaaatcaaaacattcaaacggcgcgatgggagtttagggaaatacaaacgaattacaattgcggataagtcaggaactattcgtatgactttatgggacgatttggctgaattagatgtaaaagttggagatgttattaaaattgaacgcgcacgggcacgtaaatggcgaaataatttagagttgagttcaacatctgaaactaagattaaaaaattagaaaactatgaaggagaacttccagagattaaagatacctacaatattggtgagctgagtcctggaatgacagcaacatttgaaggagaagttatctcagctcttccaatcaaagaatttaaacgtgctgatggtagtattggaaaattaaaatcatttattgttcgcgatgagacaggaagtattcgcgttaccttatgggataatcttacagatatcgatgttggtcgtggagattacgttcgtgttcggggctatatccgggaaggttattatgggggtttagaatgcaccgcaaattatgtagagattttaaaaaaaggagaaaaaatagagagtgaagaagtaaatattgaggatttaacaaaatatgaagatggagaactggtgagtgttaaaggtcgagttattgccatcagtaataaaaaaagcgtagatttggatggagagattgcaaaggttcaagatattatcttagataacggcactggtcgagttcgtgtttcattttggcggggaaaaactgctttattggaaaatatcaaagaaggggacttagttcgtatcacaaactgtcgcgttaagacgttttatgatcgtgaaggaaataaacggactgatttagttgccacattagaaacagaagttattaaagatgaaaacattgaagctccagagtatgagctgaaatattgcaaaattgaagatatttataatcgcgatgttgactggaacgatataaatttaatcgctcaagttgttgaggattatggagttaatgaaattgaatttgaagataaggttcgtaaagtacgcaatttattgttagaagatggaactggacgtattcggttgagtttatgggatgatttggctgaaattgagattaaagaaggagatattgtagaaattttacatgcctatgctaaggagcggggagattatatcgatttggttattggaaaatatggacgaattattatcaatccagaaggggttgaaatcaaaaccaatcgtaagtttattgcagatattgaagacggagaaactgttgaagttcgcggggctgtagttaagatcttgagtgacactctctttctttatttatgcccaaattgtcgtaagcgggttgtagagattgatggaatttataactgccctatttgtggagatgttgagccagaagagattttacgattgaattttgttgtagatgatgggactggaactttattatgtcgggcttatgatcgccgtgttgagaagatgttaaaaatgaatcgggaggagttaaagaaccttactatcgaaatggtggaagatgaaattttaggggaagagtttgttttgtatggaaatgttcgagtagagaatgatgaattaattatggttgttcgtcgcgttaatgatgtagatgttgagaaagaaattcgtatcttggaggaaatggaataa

SEQ ID NO: 112 - DNA, 60 bp, Unknown
  Mja F1
atggtaggagattatgaacgttttaaacaactcaaaaaaaaggttgctgaagcattgaat

SEQ ID NO: 113 - DNA, 60 bp, Unknown
  Mja F2
gataaaaaaattgaagaaaacggaggaatcattttgaaagatgctgcattaatgatgatt

SEQ ID NO: 114 - DNA, 60 bp, Unknown
  Mja F3
aaaaatgatgaagaatttttaattagtgatattgaagagggacagattggcgttgagatc

SEQ ID NO: 115 - DNA, 60 bp, Unknown
  Mja F4
aaaacattcaaacggcgcgatgggagtttagggaaatacaaacgaattacaattgcggat

SEQ ID NO: 116 - DNA, 60 bp, Unknown
  Mja F5
gacgatttggctgaattagatgtaaaagttggagatgttattaaaattgaacgcgcacgg

SEQ ID NO: 117 - DNA, 60 bp, Unknown
  Mja F6
agttcaacatctgaaactaagattaaaaaattagaaaactatgaaggagaacttccagag

SEQ ID NO: 118 - DNA, 60 bp, Unknown
  Mja F7
agtcctggaatgacagcaacatttgaaggagaagttatctcagctcttccaatcaaagaa

SEQ ID NO: 119 - DNA, 60 bp, Unknown
  Mja F8
ttaaaatcatttattgttcgcgatgagacaggaagtattcgcgttaccttatgggataat

SEQ ID NO: 120 - DNA, 60 bp, Unknown
  Mja F9
tacgttcgtgttcggggctatatccgggaaggttattatgggggtttagaatgcaccgca

SEQ ID NO: 121 - DNA, 60 bp, Unknown
  Mja F10
aaaatagagagtgaagaagtaaatattgaggatttaacaaaatatgaagatggagaactg

SEQ ID NO: 122 - DNA, 60 bp, Unknown
  Mja F11
agtaataaaaaaagcgtagatttggatggagagattgcaaaggttcaagatattatctta

SEQ ID NO: 123 - DNA, 60 bp, Unknown
  Mja F12
ttttggcggggaaaaactgctttattggaaaatatcaaagaaggggacttagttcgtatc

SEQ ID NO: 124 - DNA, 60 bp, Unknown
  Mja F13
cgtgaaggaaataaacggactgatttagttgccacattagaaacagaagttattaaagat

SEQ ID NO: 125 - DNA, 60 bp, Unknown
  Mja F14
aaatattgcaaaattgaagatatttataatcgcgatgttgactggaacgatataaattta

SEQ ID NO: 126 - DNA, 60 bp, Unknown
  Mja F15
aatgaaattgaatttgaagataaggttcgtaaagtacgcaatttattgttagaagatgga

SEQ ID NO: 127 - DNA, 60 bp, Unknown
  Mja F16
gatttggctgaaattgagattaaagaaggagatattgtagaaattttacatgcctatgct

SEQ ID NO: 128 - DNA, 60 bp, Unknown
  Mja F17
attggaaaatatggacgaattattatcaatccagaaggggttgaaatcaaaaccaatcgt

SEQ ID NO: 129 - DNA, 60 bp, Unknown
  Mja F18
actgttgaagttcgcggggctgtagttaagatcttgagtgacactctctttctttattta

SEQ ID NO: 130 - DNA, 60 bp, Unknown
  Mja F19
attgatggaatttataactgccctatttgtggagatgttgagccagaagagattttacga

SEQ ID NO: 131 - DNA, 60 bp, Unknown
  Mja F20
actttattatgtcgggcttatgatcgccgtgttgagaagatgttaaaaatgaatcgggag

SEQ ID NO: 132 - DNA, 60 bp, Unknown
  Mja F21
gaagatgaaattttaggggaagagtttgttttgtatggaaatgttcgagtagagaatgat

SEQ ID NO: 133 - DNA, 48 bp, Unknown
  Mja F22
gatgtagatgttgagaaagaaattcgtatcttggaggaaatggaataa

SEQ ID NO: 134 - DNA, 60 bp, Unknown
  Mja R1
ttcaatttttttatcaatcatccgatctaattcctcctcactaatattcaatgcttcagc

SEQ ID NO: 135 - DNA, 60 bp, Unknown
  Mja R2
ttcttcatcatttttttcttctccataaactccatgttcttttgcaatcatcattaatgc

SEQ ID NO: 136 - DNA, 60 bp, Unknown
  Mja R3
ccgtttgaatgttttgatttcagagatatcagtaataactccagtgatctcaacgccaat

SEQ ID NO: 137 - DNA, 60 bp, Unknown
  Mja R4
ttcagccaaatcgtcccataaagtcatacgaatagttcctgacttatccgcaattgtaat

SEQ ID NO: 138 - DNA, 60 bp, Unknown
  Mja R5
ttcagatgttgaactcaactctaaattatttcgccatttacgtgcccgtgcgcgttcaat

SEQ ID NO: 139 - DNA, 60 bp, Unknown
  Mja R6
tgtcattccaggactcagctcaccaatattgtaggtatctttaatctctggaagttctcc

SEQ ID NO: 140 - DNA, 60 bp, Unknown
  Mja R7
aataaatgattttaattttccaatactaccatcagcacgtttaaattctttgattggaag

SEQ ID NO: 141 - DNA, 60 bp, Unknown
  Mja R8
ccgaacacgaacgtaatctccacgaccaacatcgatatctgtaagattatcccataaggt

SEQ ID NO: 142 - DNA, 60 bp, Unknown
  Mja R9
ttcactctctattttttctcctttttttaaaatctctacataatttgcggtgcattctaa

SEQ ID NO: 143 - DNA, 60 bp, Unknown
  Mja R10
gctttttttattactgatggcaataactcgacctttaacactcaccagttctccatcttc

SEQ ID NO: 144 - DNA, 60 bp, Unknown
  Mja R11
ttttccccgccaaaatgaaacacgaactcgaccagtgccgttatctaagataatatcttg

SEQ ID NO: 145 - DNA, 60 bp, Unknown
  Mja R12
tttatttccttcacgatcataaaacgtcttaacgcgacagtttgtgatacgaactaagtc

SEQ ID NO: 146 - DNA, 60 bp, Unknown
  Mja R13
aattttgcaatatttcagctcatactctggagcttcaatgttttcatctttaataacttc

SEQ ID NO: 147 - DNA, 60 bp, Unknown
  Mja R14
aaattcaatttcattaactccataatcctcaacaacttgagcgattaaatttatatcgtt

SEQ ID NO: 148 - DNA, 60 bp, Unknown
  Mja R15
aatttcagccaaatcatcccataaactcaaccgaatacgtccagttccatcttctaacaa

SEQ ID NO: 149 - DNA, 60 bp, Unknown
  Mja R16
tccatattttccaataaccaaatcgatataatctccccgctccttagcataggcatgtaa

SEQ ID NO: 150 - DNA, 60 bp, Unknown
  Mja R17
gcgaacttcaacagtttctccgtcttcaatatctgcaataaacttacgattggttttgat

SEQ ID NO: 151 - DNA, 60 bp, Unknown
  Mja R18
ataaattccatcaatctctacaacccgcttacgacaatttgggcataaataaagaaagag

SEQ ID NO: 152 - DNA, 60 bp, Unknown
  Mja R19
ccgacataataaagttccagtcccatcatctacaacaaaattcaatcgtaaaatctcttc

SEQ ID NO: 153 - DNA, 60 bp, Unknown
  Mja R20
taaaatttcatcttccaccatttcgatagtaaggttctttaactcctcccgattcatttt

SEQ ID NO: 154 - DNA, 60 bp, Unknown
  Mja R21
ctcaacatctacatcattaacgcgacgaacaaccataattaattcatcattctctactcg

SEQ ID NO: 155 - DNA, 33 bp, Unknown
  Mja  R22
ttattccatttcctccaagatacgaatttcttt

SEQ ID NO: 156 - DNA, 37 bp, Unknown
  Mja F
gctgccatggtaggagattatgaacgttttaaacaac

SEQ ID NO: 157 - DNA, 34 bp, Unknown
  Mja R
gctcctcgagttattccatttcctccaagatacg

SEQ ID NO: 158 - DNA, 255 bp, Unknown
  TOPO TA Clone #1
  misc_feature (72)..(72): n is a, c, g, or t/u
  misc_feature (93)..(93): n is a, c, g, or t/u
  misc_feature (100)..(100): n is a, c, g, or t/u
  misc_feature (122)..(122): n is a, c, g, or t/u
  misc_feature (142)..(142): n is a, c, g, or t/u
  misc_feature (148)..(148): n is a, c, g, or t/u
  misc_feature (173)..(173): n is a, c, g, or t/u
  misc_feature (185)..(185): n is a, c, g, or t/u
  misc_feature (189)..(190): n is a, c, g, or t/u
  misc_feature (195)..(195): n is a, c, g, or t/u
  misc_feature (197)..(197): n is a, c, g, or t/u
  misc_feature (222)..(222): n is a, c, g, or t/u
  misc_feature (225)..(225): n is a, c, g, or t/u
  misc_feature (229)..(229): n is a, c, g, or t/u
  misc_feature (235)..(235): n is a, c, g, or t/u
  misc_feature (246)..(246): n is a, c, g, or t/u
  misc_feature (248)..(250): n is a, c, g, or t/u
tttaacaagcacaatataataaaattgaaggtaggacctttaaataaaagaaaccggtcgccgaaagcaaanaacgaaagggaggaagtgatnaaaaagnaaaggaaaaaagaggcaatagncaaaggttggtgtggggggngattangggattgaataatagacgtgagacnagaacaaacccnaggnnaaaancnaattctacaaatatggatgagactnacnacgnctcaangataaatgaanannnaggaa

SEQ ID NO: 159 - DNA, 246 bp, Unknown
  TOPO TA Clone #2
  misc_feature (6)..(6): n is a, c, g, or t/u
  misc_feature (23)..(23): n is a, c, g, or t/u
  misc_feature (26)..(27): n is a, c, g, or t/u
  misc_feature (29)..(29): n is a, c, g, or t/u
  misc_feature (36)..(36): n is a, c, g, or t/u
  misc_feature (38)..(39): n is a, c, g, or t/u
  misc_feature (41)..(41): n is a, c, g, or t/u
  misc_feature (62)..(63): n is a, c, g, or t/u
  misc_feature (69)..(69): n is a, c, g, or t/u
  misc_feature (73)..(73): n is a, c, g, or t/u
  misc_feature (75)..(75): n is a, c, g, or t/u
  misc_feature (77)..(78): n is a, c, g, or t/u
  misc_feature (81)..(81): n is a, c, g, or t/u
  misc_feature (83)..(84): n is a, c, g, or t/u
  misc_feature (107)..(107): n is a, c, g, or t/u
  misc_feature (170)..(170): n is a, c, g, or t/u
  misc_feature (199)..(200): n is a, c, g, or t/u
  misc_feature (202)..(203): n is a, c, g, or t/u
  misc_feature (206)..(206): n is a, c, g, or t/u
  misc_feature (210)..(210): n is a, c, g, or t/u
aattcncgttccacgtttaactnagnngntaccagncnngngaagcttttaagcaacgcaannaatgantaananannaangnnaaaaccccggaagacggaaaggntgtgtggggaggggttagaaggattcattaatgaaatgaaaagaaagggttaaacaggggaancggcaattttgcagatatccatcacagtnngnnccngtcnagaatgcatcacaccccccaattcgcactataaaga

SEQ ID NO: 160 - DNA, 13 bp, Unknown
  Priming site 13619-13631
caagcaggggagg

SEQ ID NO: 161 - DNA, 20 bp, Unknown
  Priming site 13951 R
cctgtggcaagcaggggagg

SEQ ID NO: 162 - DNA, 279 bp, Unknown
  Control
  misc_feature (12)..(12): n is a, c, g, or t/u
  misc_feature (18)..(19): n is a, c, g, or t/u
  misc_feature (22)..(22): n is a, c, g, or t/u
  misc_feature (27)..(28): n is a, c, g, or t/u
  misc_feature (39)..(41): n is a, c, g, or t/u
  misc_feature (43)..(44): n is a, c, g, or t/u
  misc_feature (55)..(55): n is a, c, g, or t/u
  misc_feature (57)..(57): n is a, c, g, or t/u
  misc_feature (60)..(60): n is a, c, g, or t/u
  misc_feature (66)..(67): n is a, c, g, or t/u
  misc_feature (70)..(70): n is a, c, g, or t/u
  misc_feature (72)..(72): n is a, c, g, or t/u
  misc_feature (74)..(75): n is a, c, g, or t/u
  misc_feature (78)..(78): n is a, c, g, or t/u
  misc_feature (94)..(94): n is a, c, g, or t/u
  misc_feature (102)..(102): n is a, c, g, or t/u
  misc_feature (105)..(105): n is a, c, g, or t/u
  misc_feature (112)..(112): n is a, c, g, or t/u
  misc_feature (115)..(115): n is a, c, g, or t/u
  misc_feature (121)..(122): n is a, c, g, or t/u
  misc_feature (125)..(125): n is a, c, g, or t/u
  misc_feature (130)..(130): n is a, c, g, or t/u
  misc_feature (133)..(133): n is a, c, g, or t/u
  misc_feature (139)..(139): n is a, c, g, or t/u
  misc_feature (141)..(142): n is a, c, g, or t/u
  misc_feature (158)..(158): n is a, c, g, or t/u
  misc_feature (160)..(160): n is a, c, g, or t/u
  misc_feature (162)..(163): n is a, c, g, or t/u
  misc_feature (165)..(165): n is a, c, g, or t/u
  misc_feature (168)..(168): n is a, c, g, or t/u
  misc_feature (178)..(179): n is a, c, g, or t/u
  misc_feature (183)..(184): n is a, c, g, or t/u
  misc_feature (187)..(190): n is a, c, g, or t/u
  misc_feature (193)..(193): n is a, c, g, or t/u
  misc_feature (195)..(195): n is a, c, g, or t/u
  misc_feature (201)..(203): n is a, c, g, or t/u
  misc_feature (211)..(212): n is a, c, g, or t/u
  misc_feature (215)..(215): n is a, c, g, or t/u
  misc_feature (219)..(220): n is a, c, g, or t/u
  misc_feature (222)..(222): n is a, c, g, or t/u
  misc_feature (227)..(228): n is a, c, g, or t/u
  misc_feature (231)..(231): n is a, c, g, or t/u
  misc_feature (235)..(236): n is a, c, g, or t/u
  misc_feature (238)..(238): n is a, c, g, or t/u
  misc_feature (244)..(244): n is a, c, g, or t/u
  misc_feature (246)..(246): n is a, c, g, or t/u
  misc_feature (248)..(248): n is a, c, g, or t/u
  misc_feature (252)..(252): n is a, c, g, or t/u
  misc_feature (258)..(258): n is a, c, g, or t/u
  misc_feature (261)..(261): n is a, c, g, or t/u
  misc_feature (264)..(264): n is a, c, g, or t/u
  misc_feature (268)..(268): n is a, c, g, or t/u
  misc_feature (272)..(272): n is a, c, g, or t/u
  misc_feature (275)..(275): n is a, c, g, or t/u
  misc_feature (278)..(278): n is a, c, g, or t/u
tcggcgctttcnaattannganttttnnttttggactgnnngnngcccccgcccngncgncgacannatntncnntantgcatctacgctgttngacccacncgnggggttnttngtgacnnctngcatntgngtgacntnngctgacaagggccatntncnntnaanctatatattnncccnnacnnnntcngngcacannnttcttcanntancgtnngnttctnnctntctnnanactgtntntnatgnacgaanagngangacncgtnccntcnc

SEQ ID NO: 163 - DNA, 249 bp, Unknown
  With 50ng MjaSSB
  misc_feature (14)..(14): n is a, c, g, or t/u
  misc_feature (23)..(23): n is a, c, g, or t/u
  misc_feature (49)..(49): n is a, c, g, or t/u
  misc_feature (112)..(112): n is a, c, g, or t/u
  misc_feature (141)..(141): n is a, c, g, or t/u
  misc_feature (153)..(153): n is a, c, g, or t/u
  misc_feature (156)..(156): n is a, c, g, or t/u
  misc_feature (173)..(173): n is a, c, g, or t/u
  misc_feature (185)..(185): n is a, c, g, or t/u
  misc_feature (197)..(198): n is a, c, g, or t/u
  misc_feature (200)..(200): n is a, c, g, or t/u
  misc_feature (206)..(206): n is a, c, g, or t/u
  misc_feature (220)..(220): n is a, c, g, or t/u
  misc_feature (226)..(226): n is a, c, g, or t/u
  misc_feature (240)..(240): n is a, c, g, or t/u
  misc_feature (243)..(243): n is a, c, g, or t/u
  misc_feature (248)..(248): n is a, c, g, or t/u
tgccagtcggatancattccccnctctagaaataattctgtttaacttnaagaaggagatatacatatgaaatacctgctgccgaccgctgctgctggtctgctgctcctcnctgcccagccggcgagggccatggatatnggaattaattcncanccgaattcggggacaantttgtacaaaanatcatgctctcnngntcgcanacgttttgcagcancagtcntttcacgttccctnacntatcng

SEQ ID NO: 164 - DNA, 249 bp, Unknown
  With 100ng MjaSSB
  misc_feature (3)..(4): n is a, c, g, or t/u
  misc_feature (9)..(9): n is a, c, g, or t/u
  misc_feature (22)..(23): n is a, c, g, or t/u
  misc_feature (30)..(31): n is a, c, g, or t/u
  misc_feature (42)..(43): n is a, c, g, or t/u
  misc_feature (52)..(52): n is a, c, g, or t/u
  misc_feature (59)..(59): n is a, c, g, or t/u
  misc_feature (143)..(143): n is a, c, g, or t/u
  misc_feature (239)..(240): n is a, c, g, or t/u
  misc_feature (243)..(244): n is a, c, g, or t/u
  misc_feature (249)..(249): n is a, c, g, or t/u
aanncgacncactatatggccnntctaganntcattacccgnnagatctctnaagaganatatacatatgaaatacctgctgcccgaccgctgctgctggtctgctgctcctcgctgcccatccggcgatggccatggatgtntggaattaattcggatccgaattcggggacaagtttgtacaaaaaagcaggctctcaggtcgcagacgttttgcagcagcagtcgcttcacgtttnnttnngtatn